(19) United States
(12) Patent Application Publication
(10) Pub. No.: US 2024/0336972 A1
(43) Pub. Date: Oct. 10, 2024

(54) METHODS FOR SELECTING MELANOMA PATIENTS FOR THERAPY AND METHODS OF REDUCING OR PREVENTING MELANOMA METASTASIS

(71) Applicant: Oregon Health & Science University, Portland, OR (US)

(72) Inventor: Wesley Yu, Portland, OR (US)

(73) Assignee: Oregon Health & Science University, Portland, OR (US)

(21) Appl. No.: 18/292,884

(22) PCT Filed: Jan. 5, 2022
(86) PCT No.: PCT/US22/11264

§ 371 Date: Jan. 26, 2024

Related U.S. Application Data
(60) Provisional application No. 63/227,930, filed on Jul. 30, 2021.

(51) Int. Cl. C12Q 1/6886; A61K 31/22; A61K 31/366; A61K 31/405; A61K 31/455; A61K 31/47; A61K 31/505; A61K 45/06; A61P 35/00

(52) U.S. Cl. CPC C12Q 1/6886; A61K 31/22; A61K 31/366; A61K 31/405; A61K 31/455; A61K 31/47; A61K 31/505; A61K 45/06; A61P 35/00; C12Q 2600/106; C12Q 2600/156; C12Q 2600/158

(57) ABSTRACT

The present disclosure provides the use of inhibitor(s) of an activity of HMG-CoA reductase (HMGCR) (a statin) to reduce and/or prevent future metastasis and prolong survival inpatients diagnosed with melanoma, for instance determined to be at high-risk of melanoma progression, such as melanoma metastasis. Also disclosed are methods for treating patients with a statin. Optionally, described methods involve measurement of a gene expression profile signature in the primary melanoma.

### CROSS-REFERENCE TO RELATED APPLICATION

This application is the 371 National Phase of International Application No. PCT/US22/11264, filed on Jan. 5, 2022, which claims priority to and the benefit of the earlier filing of U.S. Provisional Application No. 63/227,930, filed on Jul. 30, 2021; each of which is incorporated by reference herein in its entirety.

### FIELD OF THE DISCLOSURE

The present disclosure relates to the use of biometrics, such as gene expression measurements, to select melanoma patients for statin therapy in order to treat or prevent metastasis. It also relates to use of the statin class of compounds for the prevention of metastasis of melanoma.

### BACKGROUND OF THE DISCLOSURE

Cutaneous melanoma is an aggressive form of skin cancer with over 100,000 US cases in 2021 (Siegel et al., CA Cancer J Clin. 2021; 71(1):7-33. doi:10.3322/caac.21654); annually, melanoma causes over 7000 deaths. Tumor stage is determined by histopathologic and clinical factors, which include the Breslow depth of the tumor, ulceration status, spread of disease from the primary tumor to lymph nodes, and the presence of metastasis (Siegel et al., CA Cancer J Clin. 2021; 71(1):7-33. doi:10.3322/caac.21654). These factors are summarized into an overall stage denoted by a Roman numeral from 0 to IV, with Stage 0 disease being the earliest and Stage IV being the most advanced (Siegel et al., CA Cancer J Clin. 2021; 71(1):7-33. doi:10.3322/caac.21654). Patients within a stage should theoretically have similar outcomes, but there is still significant heterogeneity within each stage. For example, most patients with Stage I disease will be cured by surgery, but a significant minority (5-10%) may go on to develop metastasis at a later time.

Up to one-third of melanoma deaths are caused by early, localized melanomas (AJCC Stage 1-2) that progress to metastasis despite adequate surgical treatment (Whiteman et al., J Invest Dermatol. 2015; 135(4):1190-1193. doi:10.1038/jid.2014.452). In addition, metastatic disease accounts for over 40% of the annual economic burden of melanoma. A therapy to prevent progression to metastasis in patients determined to be at high-risk of metastasis could save lives and reduce overall healthcare costs.

Gene expression signatures have been identified that predict which melanomas will recur or metastasize (Gerami et al., Clin Cancer Res. 2015. doi:10.1158/1078-0432.CCR-13-3316; Zager et al., BMC Cancer. 2018. doi:10.1186/s12885-018-4016-3; Greenhaw et al., Dermatologic Surg. 2018. doi:10.1097/DSS.0000000000001588). Gene expression profiling has been used to better identify patients at risk of future melanoma metastasis. Examples include the DecisionDx-Melanoma test (see U.S. Patent Application Publication No. US20200362419A1) and the Merlin Assay (see WO2020022895A2). These tests generally stratify patients into either high- or low-risk groups, and are used for prognosis. No treatment exists for patients with melanoma identified by these tests as being at high-risk for metastasis.

The clinical utility of gene expression profiling (GEP) has been limited by the lack of proven treatments for patients who are determined to be at high-risk for future progression or metastasis. A recent melanoma expert consensus statement determined that there is no clinical action associated with the result (Grossman et al., JAMA Dermatology. 2020. doi:10.1001/jamadermatol.2020.1729; Marchetti et al., J Am Acad Dermatol. 2019; 80(6): e161-el62. doi:10.1016/j.jaad.2018.11.063; Marchetti et al., JAMA Dermatology. 2020; 156(9):953. doi:10.1001/jamadermatol.2020.1731). Gene expression tests have never been used to select melanoma patients for treatments to reduce future metastasis and the finding that they could be used to select patients for treatment would be extremely novel and impactful. One recent expert review states that a “major challenge of GEP testing [is] determining [ . . . ] how results should affect patient management. It is not currently known whether a high-risk GEP classification is associated with improved response to [ . . . ] systemic therapies.” (Grossman et al., Melanoma Manag. 2019; 6(4):MMT32. doi:10.2217/mmt-2019-0016). The National Comprehensive Cancer Network@ (NCCN) Clinical Practice Guideline (CPG) in Oncology® recommendations for the clinical staging and workup of cutaneous melanoma (V2. 2019) states: “While there is interest in newer prognostic molecular techniques such as gene expression profiling to differentiate melanomas at low- versus high-risk for metastasis, routine (baseline) genetic testing of primary cutaneous melanomas [ . . . ] is not recommended outside of a clinical study (trial).” The updated 2021 NCCN Clinical Practice Guidelines reiterate this sentiment, stating: “the impact of these tests on treatment outcomes or follow-up schedules has not been established.” The 2020 Medical Policy of multiple insurers such as Anthem BlueCross and Wellmark state that GEP testing “may improve our ability to determine prognosis in individuals with primary cutaneous melanoma, but how this would alter disease management and health outcomes remains unclear.” (Anthem BlueCross Medical Policy Regarding Gene Expression Profiling of Melanomas. Available online at anthem.com/dam/medpolicies/abc/active/policies/mp_pw_c148391.html. Accessed Jun. 17, 2021). The 2019 guideline by the American Academy of Dermatology (AAD) regarding care for the management of primary cutaneous melanoma states: “Evidence is lacking that molecular classification should be used to alter patient management.” (Swetter et al., J Am Acad Dermatol. 2019; 80(1):208-250. doi:10.1016/j.jaad.2018.08.055).

Previous studies on statins in melanoma have focused on melanoma initiation and primary prevention rather than metastasis prevention and have had mixed results. Two large cardiovascular trials demonstrated a small reduction in melanoma incidence with statin use, but this effect was not observed in the Women's Health Initiative or two Dutch epidemiologic studies (Splichal et al., Semin Thromb Hemost. 2003. doi:10.1055/s-2003-40964; Rubins et al., N Engl J Med. 1999. doi:10.1056/NEJM199908053410604; Jagtap et al., Cancer. 2012. doi:10.1002/cncr.27497). Two meta-analyses also demonstrated no reduction in melanoma incidence with statin use, and a randomized controlled trial of lovastatin for melanoma prevention did not identify any significant decreases in melanocytic atypia or other melanoma initiation markers (Bonovas et al., Eur J Epidemiol. 2010. doi:10.1007/s10654-009-9396-x; Freeman et al., J Natl Cancer Inst. 2006. doi:10.1093/jnci/djj412; Linden et al., Cancer Prev Res (Phila). 2014; 7(5):496-504. doi:10.1158/1940-6207.CAPR-13-0189). Recently, a Mendelian randomization analysis using the UK Biobank demonstrated that individuals with variants in the HMGCR region, which represent proxies for statin use, had decreased overall cancer risk but did not reach statistical significance for any site-specific cancers (Carter et al., medRxiv. 2020. doi:https://doi.org/10.1101/2020.02.28.20028902). In summary, the current evidence available in the literature and the public domain suggest that statins have little to no effect on melanoma initiation and progression.

Several studies of in vitro and animal models have suggested that statins might decrease tumor cell migration, decrease cell adhesion, and increase immunogenicity and prevent progression of melanoma metastasis (Collisson et al., Mol Cancer Ther. 2003; Pich et al., Front Immunol. 2013. doi:10.3389/fimmu.2013.0006; Kidera et al., J Exp Clin Cancer Res. 2010. doi:10.1186/1756-9966-29-127; Zanfardino et al., Int J Oncol. 2013. doi:10.3892/ijo.2013.2126). However, when these studies were followed up in large human clinical cohorts, the effect was not observed and there was no statistically significance decrease in metastasis (Koomen et al., Eur J Cancer. 2007. doi:10.1016/j.ejca.2007.09.004). Thus, statins are currently not used in metastasis prevention because there is no evidence that they have any treatment effect in unselected populations of melanoma patients; in fact, past attempts to find any beneficial effect of statins in treatment of melanoma have failed. The finding that statins could have a beneficial effect on selected melanoma patients would thus be novel and surprising.

There exists an unmet need in the art for methods of selecting melanoma patients for therapy, and for methods of preventing melanoma metastasis.

### SUMMARY OF THE DISCLOSURE

There is a clear need in the art for a treatment to prevent metastasis and progression of melanoma determined to be at high-risk of future metastasis.

The present disclosure enables the use of statin therapy to reduce and/or prevent future metastasis and prolong survival in patients determined to be at high-risk of melanoma progression, such as melanoma metastasis. A treatment that can prevent future metastasis of high-risk melanomas, as described herein, is a significant advance in the field.

Also disclosed herein are methods for treating patients with a statin (that is, an inhibitor of an activity of HMG-CoA reductase (HMGCR)), the methods involving: (1) determining a melanoma patient to be at high risk of future metastasis through a combination of genetic testing and optionally clinicopathologic factors; and (2) treating those selected high-risk patients with a HMGCR inhibitor, such as a statin.

The gene expression signatures useful in the provided methods may include two or more of the genes listed in Table 5. For instance, a gene expression signature may include those genes listed in Table 1, Table 2, Table 3, Table 4, or any combination thereof. The directionality of change of gene expression level that is indicative of a high-risk for metastatic melanoma is provided in Table 5, as well as each of Tables 1-4.

One embodiment is a method of reducing risk of future melanoma metastasis and/or progression in a subject with high-risk primary melanoma, which method includes selecting a subject with high-risk primary melanoma, and administering to the selected subject a composition including an inhibitor of 3-Hydroxy-3-Methylglutaryl-CoA Reductase (HMGCR) (a statin, such as fluvastatin, pitavastatin, atorvastatin, simvastatin, lovastatin, rosuvastatin, or pravastatin). In examples of this method, the primary melanoma is determined to be high-risk based on one or more genetic features. By way of example, the genetic features may include: a mutation in any of the genes listed in Table 5; or a change in expression of any of the genes listed in Table 5, compared to expression of the gene in a control or reference sample.

In examples of these method, in the genetic features are evaluated by gene expression profiling (GEP). For instance, the GEP in example methods includes at least one of the genes listed in Table 5; at least five of the genes listed in Table 5; at least ten of the genes listed in Table 5; at least twenty six (26) of the genes listed in Table 5. In additional examples, the gene expression profile includes at least 30, at least 40, at least 50, or at least 60, at least 70, at least 80, at least 90, at least 100, at least 125, or more than 120 of the genes listed in Table 5. One specific example includes a method wherein the gene expression profile includes all of the genes ANGPT2, ABCC12, ACOT1, ADAM12, AGBL4, AGXT, AIM2, ALK, ANGPT1, ANGPTL7, ANK1, AQP3, ARG1, ARRDC1, ART1, BAP1, BIN1, BMP2, BMX, BTG1, C8G, CACNG4, CAMK2B, CASQ1, CCR3, CCR5, CDC5L, CENPQ, CETP, CLCA2, CLIC5, COL24A1, CPN2, CRABP2, CST6, CTAGE1, CTCFL, CUL7, CXCL14, CXCL8/IL8, DIO3OS, DMAP1, DOCK3, DPEP3, DSC1, DYSF, EEF1A2, EHBP1L1, EIF1B, ERGIC2, F7, FASLG, FGF2, FLOT1, FLVCR2, FNBP1L, FREM2, GABBR2, GBP5, GCH1, GDF15, GFRA1, GJA1, GLDC, GPR39, GPR63, GRAMD1B, HEPACAM, HHATL, HMGCR, HNF4A, HTR3B, ID2, IFNG, ITGB3, JARID2, JPH3, KCNT1, KIF19, KLC4, KLHDC8A, KNDC1, KRT14, KRT6B, KRTAP19-6, KSR2, KYNU, LAG3, LAMB1, LGALSI, LGI4, LRRC31, LTA4H, LOXL4, MCF2, ME3, MFSD6L, MGP, MIR222, MLANA, MLF1, MTUS2, NFYA, NPPC, NTRK1, NXPH1, OCIAD2, OR52K1, OR5AK2, P2RY14, PARP11, PHEX, PIK3R6, PLA2G2D, PLAT, PLCZ1, PPL, PRF1, PRKCB, PROCA1, PTPRG, RAB15, RBBP4, RBM23, RGS7, RNF213, ROBO1, RP1, RPL7L1, S100A8, S100A9, S1PR1, SAP130, SERPINE2, SFMBT2, SIGLEC12, SLC17A3, SLC29A1, SLCO5A1, SLIT1, SOCS1, SOX4, SPAG6, SPP1, SPRR1B, STMN1, TACSTD2, TAS2R60, TDRD12, TFF2, TGFB1, TGFBRI, TLR4, TMCC3, TNFRSF10A, TP53, TRIM22, TRIM29, TRNAU1AP, TTN, TUBB, TYRP1, UTS2, VAMP5, VEGFA, VEGFC, VEGFD, WDR49, WIF1, ZIM2, ZNF560, and ZNF697.

In yet additional embodiments of a method of reducing risk of future melanoma metastasis and/or progression in a subject with high-risk primary melanoma, the gene expression profile: includes the genes in Table 1 (BAP1, MGP, SPP1, CXCL14, CLCA2, S100A8, BTG1, SAP130, ARG1, KRT6B, GJA1, ID2, EIF1B, S100A9, CRABP2, KRT14, ROBO1, RBM23, TACSTD2, DSC1, SPRR1B, TRIM29, AQP3, TYRP1, PPL, LTA4H, and CST6); consists essentially of the genes BAP1, MGP, SPP1, CXCL14, CLCA2, S100A8, BTG1, SAP130, ARG1, KRT6B, GJA1, ID2, EIF1B, S100A9, CRABP2, KRT14, ROBO1, RBM23, TACSTD2, DSC1, SPRR1B, TRIM29, AQP3, TYRP1, PPL, LTA4H, and CST6; consists of the genes in BAP1, MGP, SPP1, CXCL14, CLCA2, S100A8, BTG1, SAP130, ARG1, KRT6B, GJA1, ID2, EIF1B, S100A9, CRABP2, KRT14, ROBO1, RBM23, TACSTD2, DSC1, SPRR1B, TRIM29, AQP3, TYRP1, PPL, LTA4H, and CST6; includes the genes in Table 2 (CXCL8, ITGB3, LAMB1, PLAT, and TP53); consists essentially of the genes CXCL8, ITGB3, LAMB1, PLAT, and TP53; consists of the genes CXCL8, ITGB3, LAMB1, PLAT, and TP53; includes the genes in Table 3 (ANGPT1, ANGPT2, BMP2, FGF2, S1 PR1, TGFB1, VEGFA, VEGFC, VEGFD, and IFNG); consists essentially of the genes ANGPT1, ANGPT2, BMP2, FGF2, S1PR1, TGFB1, VEGFA, VEGFC, VEGFD, and IFNG; consists of the genes ANGPT1, ANGPT2, BMP2, FGF2, S1PR1, TGFB1, VEGFA, VEGFC, VEGFD, and IFNG; includes the genes in Table 4 (GDF15, MLANA, PLAT, CXCL8/IL8, ITGB3, LOXL4, PRKCB, SERPINE2, ADAM12, LGALSI, and TGFBRI); consists essentially of the genes GDF15, MLANA, PLAT, CXCL8/IL8, ITGB3, LOXL4, PRKCB, SERPINE2, ADAM12, LGALSI, and TGFBRI; or consists of the genes GDF15, MLANA, PLAT, CXCL8/IL8, ITGB3, LOXL4, PRKCB, SERPINE2, ADAM12, LGALSI, and TGFBR.

In any of these embodiments, the subject may be selected for treatment based on at least one high risk clinicopathologic features selected from the group consisting of Breslow depth, histologic subtype, mitotic rate, ulceration, sentinel lymph node status, and results of imaging.

In any of these embodiments, the subject may be selected for statin treatment based on a combination of clinicopathologic features and genetic features.

Also contemplated are method embodiments that further include treating the subject with immunotherapy or chemotherapy or both, as well as method embodiments that further include treating the subject with nicotinamide or niacin or both.

Another embodiment is a method of selecting a subject afflicted with primary melanoma for treatment with a statin, the method including measurement of a gene expression profile (GEP) in the primary melanoma. For instance, the GEP in example methods includes at least one of the genes listed in Table 5; at least five of the genes listed in Table 5; at least ten of the genes listed in Table 5; at least twenty six (26) of the genes listed in Table 5. In additional examples, the gene expression profile includes at least 30, at least 40, at least 50, or at least 60, at least 70, at least 80, at least 90, at least 100, at least 125, or more than 120 of the genes listed in Table 5. One specific example includes a method wherein the gene expression profile includes all of the genes ANGPT2, ABCC12, ACOT1, ADAM12, AGBL4, AGXT, AIM2, ALK, ANGPT1, ANGPTL7, ANK1, AQP3, ARG1, ARRDC1, ART1, BAP1, BIN1, BMP2, BMX, BTG1, C8G, CACNG4, CAMK2B, CASQ1, CCR3, CCR5, CDC5L, CENPQ, CETP, CLCA2, CLIC5, COL24A1, CPN2, CRABP2, CST6, CTAGE1, CTCFL, CUL7, CXCL14, CXCL8/IL8, DIO3OS, DMAP1, DOCK3, DPEP3, DSC1, DYSF, EEF1A2, EHBP1Li, EIF1B, ERGIC2, F7, FASLG, FGF2, FLOT1, FLVCR2, FNBP1L, FREM2, GABBR2, GBP5, GCH1, GDF15, GFRA1, GJA1, GLDC, GPR39, GPR63, GRAMD1B, HEPACAM, HHATL, HMGCR, HNF4A, HTR3B, ID2, IFNG, ITGB3, JARID2, JPH3, KCNT1, KIF19, KLC4, KLHDC8A, KNDC1, KRT14, KRT6B, KRTAP19-6, KSR2, KYNU, LAG3, LAMB1, LGALSI, LGI4, LRRC31, LTA4H, LOXL4, MCF2, ME3, MFSD6L, MGP, MIR222, MLANA, MLF1, MTUS2, NFYA, NPPC, NTRK1, NXPH1, OCIAD2, OR52K1, OR5AK2, P2RY14, PARP11, PHEX, PIK3R6, PLA2G2D, PLAT, PLCZ1, PPL, PRF1, PRKCB, PROCA1, PTPRG, RAB15, RBBP4, RBM23, RGS7, RNF213, ROBO1, RP1, RPL7L1, S100A8, S100A9, S1PR1, SAP130, SERPINE2, SFMBT2, SIGLEC12, SLC17A3, SLC29A1, SLCO5A1, SLIT1, SOCS1, SOX4, SPAG6, SPP1, SPRR1B, STMN1, TACSTD2, TAS2R60, TDRD12, TFF2, TGFB1, TGFBRI, TLR4, TMCC3, TNFRSF10A, TP53, TRIM22, TRIM29, TRNAU1AP, TTN, TUBB, TYRP1, UTS2, VAMP5, VEGFA, VEGFC, VEGFD, WDR49, WIF1, ZIM2, ZNF560, and ZNF697.

In yet additional embodiments of a method of selecting a subject afflicted with primary melanoma for treatment with a statin, the gene expression profile: includes the genes in Table 1 (BAP1, MGP, SPP1, CXCL14, CLCA2, S100A8, BTG1, SAP130, ARG1, KRT6B, GJA1, ID2, EIF1B, S100A9, CRABP2, KRT14, ROBO1, RBM23, TACSTD2, DSC1, SPRR1B, TRIM29, AQP3, TYRP1, PPL, LTA4H, and CST6); consists essentially of the genes BAP1, MGP, SPP1, CXCL14, CLCA2, S100A8, BTG1, SAP130, ARG1, KRT6B, GJA1, ID2, EIF1B, S100A9, CRABP2, KRT14, ROBO1, RBM23, TACSTD2, DSC1, SPRR1B, TRIM29, AQP3, TYRP1, PPL, LTA4H, and CST6; consists of the genes in BAP1, MGP, SPP1, CXCL14, CLCA2, S100A8, BTG1, SAP130, ARG1, KRT6B, GJA1, ID2, EIF1B, S100A9, CRABP2, KRT14, ROBO1, RBM23, TACSTD2, DSC1, SPRR1B, TRIM29, AQP3, TYRP1, PPL, LTA4H, and CST6; includes the genes in Table 2 (CXCL8, ITGB3, LAMB1, PLAT, and TP53); consists essentially of the genes CXCL8, ITGB3, LAMB1, PLAT, and TP53; consists of the genes CXCL8, ITGB3, LAMB1, PLAT, and TP53; includes the genes in Table 3 (ANGPT1, ANGPT2, BMP2, FGF2, S1PR1, TGFB1, VEGFA, VEGFC, VEGFD, and IFNG); consists essentially of the genes ANGPT1, ANGPT2, BMP2, FGF2, S1PR1, TGFB1, VEGFA, VEGFC, VEGFD, and IFNG; consists of the genes ANGPT1, ANGPT2, BMP2, FGF2, S1 PR1, TGFB1, VEGFA, VEGFC, VEGFD, and IFNG; includes the genes in Table 4 (GDF15, MLANA, PLAT, CXCL8/IL8, ITGB3, LOXL4, PRKCB, SERPINE2, ADAM12, LGALSI, and TGFBRI); consists essentially of the genes GDF15, MLANA, PLAT, CXCL8/IL8, ITGB3, LOXL4, PRKCB, SERPINE2, ADAM12, LGALSI, and TGFBRI; or consists of the genes GDF15, MLANA, PLAT, CXCL8/IL8, ITGB3, LOXL4, PRKCB, SERPINE2, ADAM12, LGALSI, and TGFBR.

In any one of these methods of selecting a subject afflicted with primary melanoma for treatment with a statin, examples further include treating the subject with a statin. By way of example, the statin may include one or more of fluvastatin, pitavastatin, atorvastatin, simvastatin, lovastatin, rosuvastatin, or pravastatin. Optionally, the method may include treating the subject with immunotherapy or chemotherapy, and/or include treating the subject with nicotinamide or niacin.

Yet another provided embodiment is a method of selecting a subject afflicted with melanoma for treatment with a molecule inhibiting activity of HMGCR enzyme, the method including measurement of a gene expression signature in the primary melanoma.

In examples of any of the above embodiments, the subject afflicted with primary melanoma is selected for treatment with a statin in order to reduce likelihood of a future metastasis of the melanoma.

Also provided is a method of selecting a melanoma patient for treatment with a statin (HMGCR inhibitor), the method including: obtaining a sample from a primary cutaneous melanoma tumor in the subject; measuring gene expression levels of at least two genes listed in Table 5 in the sample; producing a gene expression profile including the gene expression levels of the at least two genes; comparing the gene expression profile to a gene expression profile of a reference training set gene expression profile, which training set gene expression profile includes the same at least two genes; and generating an indication that the primary cutaneous melanoma tumor is low-risk or high-risk of metastasis when the gene expression profile indicates that expression levels of at least one gene is altered in a predictive manner as compared to the gene expression profile of the reference training set By way of example, in embodiments of this method, measuring gene expression levels includes measurement of a level of fluorescence by a sequence detection system following RT-PCR. Optionally, in these methods of selecting a melanoma patient for treatment with a statin further includes treating the patient with a statin or other HMGCR inhibitor when the primary cutaneous melanoma is determined to be at high-risk for metastasis.

In non-limiting examples of these methods, the gene expression profile: includes the genes in Table 1 (BAP1, MGP, SPP1, CXCL14, CLCA2, S100A8, BTG1, SAP130, ARG1, KRT6B, GJA1, ID2, EIF1B, S100A9, CRABP2, KRT14, ROBO1, RBM23, TACSTD2, DSC1, SPRR1B, TRIM29, AQP3, TYRP1, PPL, LTA4H, and CST6); consists essentially of the genes BAP1, MGP, SPP1, CXCL14, CLCA2, S100A8, BTG1, SAP130, ARG1, KRT6B, GJA1, ID2, EIF1B, S100A9, CRABP2, KRT14, ROBO1, RBM23, TACSTD2, DSC1, SPRR1B, TRIM29, AQP3, TYRP1, PPL, LTA4H, and CST6; consists of the genes in BAP1, MGP, SPP1, CXCL14, CLCA2, S100A8, BTG1, SAP130, ARG1, KRT6B, GJA1, ID2, EIF1B, S100A9, CRABP2, KRT14, ROBO1, RBM23, TACSTD2, DSC1, SPRR1B, TRIM29, AQP3, TYRP1, PPL, LTA4H, and CST6; includes the genes in Table 2 (CXCL8, ITGB3, LAMB1, PLAT, and TP53); consists essentially of the genes CXCL8, ITGB3, LAMB1, PLAT, and TP53; consists of the genes CXCL8, ITGB3, LAMB1, PLAT, and TP53; includes the genes in Table 3 (ANGPT1, ANGPT2, BMP2, FGF2, S1PR1, TGFB1, VEGFA, VEGFC, VEGFD, and IFNG); consists essentially of the genes ANGPT1, ANGPT2, BMP2, FGF2, S1PR1, TGFB1, VEGFA, VEGFC, VEGFD, and IFNG; consists of the genes ANGPT1, ANGPT2, BMP2, FGF2, S1 PR1, TGFB1, VEGFA, VEGFC, VEGFD, and IFNG; includes the genes in Table 4 (GDF15, MLANA, PLAT, CXCL8/IL8, ITGB3, LOXL4, PRKCB, SERPINE2, ADAM12, LGALSI, and TGFBRI); consists essentially of the genes GDF15, MLANA, PLAT, CXCL8/IL8, ITGB3, LOXL4, PRKCB, SERPINE2, ADAM12, LGALSI, and TGFBRI; or consists of the genes GDF15, MLANA, PLAT, CXCL8/IL8, ITGB3, LOXL4, PRKCB, SERPINE2, ADAM12, LGALSI, and TGFBR.

This disclosure also provides a method of treating melanoma in a patient having a primary melanoma tumor, the method including: administering to the patient a therapeutically effective dose of an inhibitor of 3-Hydroxy-3-Methylglutaryl-CoA Reductase (HMGCR) (a statin, such as fluvastatin, pitavastatin, atorvastatin, simvastatin, lovastatin, rosuvastatin, or pravastatin). In examples, the primary melanoma tumor is a Stage 3 (regional) or Stage 4 (metastatic) melanoma.

In examples of the method of treating melanoma in a patient having a primary melanoma tumor, the method further includes measuring gene expression levels of at least two genes selected from the genes listed in Table 5 in a sample of a primary cutaneous melanoma tumor in the patient; determining a patient gene-expression profile signature including the gene expression levels of the at least two genes; comparing the patient gene-expression profile signature to a gene-expression profile of a predictive training set; determining whether the patient gene-expression profile signature of the at least two genes is altered in a predictive manner compared to the predictive training set. By way of example, the gene expression levels are in some cases measured using Polymerase Chain Reaction (PCR), Real-Time Polymerase Chain Reaction (RT-PCR), direct DNA expression in microarray, Sanger sequencing analysis, Northern blot, direct RNA expression detection serial analysis of gene expression, or next-generation RNA-sequencing.

For instance, the GEP in example methods includes at least five of the genes listed in Table 5; at least ten of the genes listed in Table 5; at least twenty six (26) of the genes listed in Table 5. In additional examples, the gene expression profile includes at least 30, at least 40, at least 50, or at least 60, at least 70, at least 80, at least 90, at least 100, at least 125, or more than 120 of the genes listed in Table 5. One specific example includes a method wherein the gene expression profile includes all of the genes ANGPT2, ABCC12, ACOT1, ADAM12, AGBL4, AGXT, AIM2, ALK, ANGPT1, ANGPTL7, ANK1, AQP3, ARG1, ARRDC1, ART1, BAP1, BIN1, BMP2, BMX, BTG1, C8G, CACNG4, CAMK2B, CASQ1, CCR3, CCR5, CDC5L, CENPQ, CETP, CLCA2, CLIC5, COL24A1, CPN2, CRABP2, CST6, CTAGE1, CTCFL, CUL7, CXCL14, CXCL8/IL8, DIO3OS, DMAP1, DOCK3, DPEP3, DSC1, DYSF, EEF1A2, EHBP1L1, EIF1B, ERGIC2, F7, FASLG, FGF2, FLOT1, FLVCR2, FNBP1L, FREM2, GABBR2, GBP5, GCH1, GDF15, GFRA1, GJA1, GLDC, GPR39, GPR63, GRAMD1B, HEPACAM, HHATL, HMGCR, HNF4A, HTR3B, ID2, IFNG, ITGB3, JARID2, JPH3, KCNT1, KIF19, KLC4, KLHDC8A, KNDC1, KRT14, KRT6B, KRTAP19-6, KSR2, KYNU, LAG3, LAMB1, LGALSI, LGI4, LRRC31, LTA4H, LOXL4, MCF2, ME3, MFSD6L, MGP, MIR222, MLANA, MLF1, MTUS2, NFYA, NPPC, NTRK1, NXPH1, OCIAD2, OR52K1, OR5AK2, P2RY14, PARP11, PHEX, PIK3R6, PLA2G2D, PLAT, PLCZ1, PPL, PRF1, PRKCB, PROCA1, PTPRG, RAB15, RBBP4, RBM23, RGS7, RNF213, ROBO1, RP1, RPL7L1, S100A8, S100A9, S1PR1, SAP130, SERPINE2, SFMBT2, SIGLEC12, SLC17A3, SLC29A1, SLCO5A1, SLIT1, SOCS1, SOX4, SPAG6, SPP1, SPRR1B, STMN1, TACSTD2, TAS2R60, TDRD12, TFF2, TGFB1, TGFBRI, TLR4, TMCC3, TNFRSF10A, TP53, TRIM22, TRIM29, TRNAU1AP, TTN, TUBB, TYRP1, UTS2, VAMP5, VEGFA, VEGFC, VEGFD, WDR49, WIF1, ZIM2, ZNF560, and ZNF697.

In yet additional embodiments of a method of treating melanoma in a patient having a primary melanoma tumor, the GEP: includes the genes in Table 1 (BAP1, MGP, SPP1, CXCL14, CLCA2, S100A8, BTG1, SAP130, ARG1, KRT6B, GJA1, ID2, EIF1B, S100A9, CRABP2, KRT14, ROBO1, RBM23, TACSTD2, DSC1, SPRR1B, TRIM29, AQP3, TYRP1, PPL, LTA4H, and CST6); consists essentially of the genes BAP1, MGP, SPP1, CXCL14, CLCA2, S100A8, BTG1, SAP130, ARG1, KRT6B, GJA1, ID2, EIF1B, S100A9, CRABP2, KRT14, ROBO1, RBM23, TACSTD2, DSC1, SPRR1B, TRIM29, AQP3, TYRP1, PPL, LTA4H, and CST6; consists of the genes in BAP1, MGP, SPP1, CXCL14, CLCA2, S100A8, BTG1, SAP130, ARG1, KRT6B, GJA1, ID2, EIF1B, S100A9, CRABP2, KRT14, ROBO1, RBM23, TACSTD2, DSC1, SPRR1B, TRIM29, AQP3, TYRP1, PPL, LTA4H, and CST6; includes the genes in Table 2 (CXCL8, ITGB3, LAMB1, PLAT, and TP53); consists essentially of the genes CXCL8, ITGB3, LAMB1, PLAT, and TP53; consists of the genes CXCL8, ITGB3, LAMB1, PLAT, and TP53; includes the genes in Table 3 (ANGPT1, ANGPT2, BMP2, FGF2, S1PR1, TGFB1, VEGFA, VEGFC, VEGFD, and IFNG); consists essentially of the genes ANGPT1, ANGPT2, BMP2, FGF2, S1PR1, TGFB1, VEGFA, VEGFC, VEGFD, and IFNG; consists of the genes ANGPT1, ANGPT2, BMP2, FGF2, S1PR1, TGFB1, VEGFA, VEGFC, VEGFD, and IFNG; includes the genes in Table 4 (GDF15, MLANA, PLAT, CXCL8/IL8, ITGB3, LOXL4, PRKCB, SERPINE2, ADAM12, LGALSI, and TGFBRI); consists essentially of the genes GDF15, MLANA, PLAT, CXCL8/IL8, ITGB3, LOXL4, PRKCB, SERPINE2, ADAM12, LGALSI, and TGFBRI; or consists of the genes GDF15, MLANA, PLAT, CXCL8/IL8, ITGB3, LOXL4, PRKCB, SERPINE2, ADAM12, LGALSI, and TGFBR.

Yet another embodiment is a method of treating melanoma in a patient having a primary melanoma tumor, the method including administering to the patient: a therapeutically effective dose of an inhibitor of 3-Hydroxy-3-Methylglutaryl-CoA Reductase (HMGCR) (a statin, such as fluvastatin, pitavastatin, atorvastatin, simvastatin, lovastatin, rosuvastatin, or pravastatin); and a therapeutically effective dose of a melanoma treatment selected from a BRAF inhibitor and an immunotherapy for treatment of melanoma. In examples of this method embodiment, the statin and the melanoma treatment are administered concurrently, or sequentially. By way of example, the BRAF inhibitor may be any recognized inhibitor of BRAF, such as Vemurafenib, dabrafenib, GDC-0879, PLX 4032, PLX-4720, PLX 4734, Sorafenib Tosylate, or a combination of trametinib and dabrafenib.

In each method embodiment provided herein, examples are included in which the subject is not treated with a RORgamma inhibitor.

### BRIEF DESCRIPTION OF THE DRAWINGS

Some of the drawings submitted herein may be better understood in color. Applicant considers the color versions of the drawings as part of the original submission and reserves the right to present color images of the drawings in later proceedings.

FIG. 1. Heatmap of the top 100 differentially expressed genes after fluvastatin treatment. K, keratin; MMP, matrix metalloproteinase. In order, the 100 illustrated genes are: ADAMTS16, CTB-47B8.1, RP11-98F14.11, AVIL, OLFML2A, RASA4, RN7SKP55, DACT1, GJB2, CEACAMI, DUSP8, CYSLTR1, CALB1, FAM83A, RN7SL865P, CAPS, BMF, PTPRH, JUP, KRT14, C10orf10, MIR210HG, AQP3, LGR6, KRT15, AKR1C1, CRISPLD2, WFDC3, ELF3, TNS4, SLCO4A1-AS1, LYPD3, PCDH1, CTD-2587H24.5, MYO15B, KLF6, AKR1C2, CLDN4, IL1B, RHOB, WNT9A, TSPAN1, NDRG1, AHNAK2, OSBP2, TMC7, TSC22D3, RP11-443P15.2, CDKN1C, SERPINB2, ADRAIB, BMP6, GJB3, HAS2, CYP27C1, ACE, GALNT9, DSC2, TRAF1, RP11-96B2.1, SNAI2, BTNL8, MMP1, ATP2B4, MAST4, RP5-1063M23.2, C11orf96, RP11-757F18.5, NGEF, SH3RF2, ABHD11-AS1, IL32, KLF2, KDR, CNN1, TCF7, HCN1, GHRL, ZRANB2-AS2, MIR600HG, MAGI2, CACNG8, AC004941.5, AC004941.5, PDE3B, ZNF385D, KCNJ15, WT1-AS, LRRC17, SCN5A, CYP24A1, EFEMPI, KCNK2, HIGD1AP1, IL7R, ST18, APLN, ANKRD1, VDR, RNF152, and THBS1.

FIG. 2. Heatmap of differentially expressed genes, illustrating that fluvastatin significantly affected the expression of genes previously shown to be involved in metastasis.

FIGS. 3A, 3B. Kaplan Meier curves showing that statin treatment is effective in melanoma patients determined to be high risk (that is, at high risk for developing metastatic melanoma) as determined by gene expression signature (FIG. 3B). Patients with a low risk gene expression profile do not benefit from statin treatment (FIG. 3A).

FIG. 4 is a schematic representation of cohort development for the analysis described in Example 4.

FIGS. 5A-5C. Kaplan Meier curves showing that statin treatment is effective in prolonging survival in melanoma patients with local (FIG. 5A), regional (FIG. 5B), and distant (FIG. 5C) disease. Local, regional, and distant melanoma diagnoses were determined by ICD10 codes.

FIG. 6. Five Year Survival After Diagnosis of Malignant Melanoma Stratified by Statin Use. This figure describes the probability of survival during the first five years following malignant melanoma diagnosis. The cohort is stratified by statin use. Patients with at least one year (including the date of melanoma diagnosis) of statin use are represented by the dashed line. Patients with no statin exposure are represented by the solid line. Time (in months from the date of diagnosis) is shown along the x-axis, with survival probability on the y-axis. The table depicts the number of individuals at risk for each 6-month interval. P-value, computed by log-rank, reflects a statistically significant difference at the 0.0001 level in survival probability, based on statin exposure.

FIG. 7. Overall Proportional Hazard Ratios During the First Year After Diagnosis. Cox proportional hazards regression modelling, with backwards model selection by Akaike Information Criterion, was used to describe the influence of multiple covariates on one year survival following diagnosis of malignant melanoma. The most influential factors were metastatic disease at diagnosis (hazard ratio (HR)=4.87) and statin use (HR=0.38). While this model was statistically significant at the 0.0001 level (by log-rank), testing demonstrated that it violated the proportional hazards assumptions as the protective effect of statins varied over time (FIG. 8), and it is included here only to provide an overall assessment of the proportional hazards.

FIG. 8. Time Varying Proportional Hazard Ratios During the First Year After Diagnosis. This figure describes the time varying factors contributing to the proportional hazards model. Effects of four of the eight covariates changed over time, with statin use and beta blocker use showing the most dramatic variance. The hazard ratio for beta blocker use varies from 0.2 to 0.5 and is only statistically significant for the first 12 weeks of the year following malignant melanoma diagnosis. The hazard ratio for statin use varies from 0.03 to 0.6 over the year, with only two of the 4-week segments not showing statistical significance. The HR for statin shows a general trend of rising over the year.

FIG. 9. Kaplan Meier curves show that the combination of statins with immunotherapy or a BRAF inhibitor is more effective than immunotherapy or a BRAF inhibitor alone. Adding statins to systemic therapy (either immunotherapy or BRAF inhibitors) leads to an increase in survival at 3 years of follow up.

### DETAILED DESCRIPTION

Described are methods of reducing risk of future melanoma metastasis and/or progression in a subject with a high-risk for metastatic primary melanoma. The methods involve identifying the subject as one with high-risk for metastatic melanoma, then administering to the subject a composition comprising an inhibitor of HMGCR (a statin). Also described are methods of selecting a subject with a melanoma for treatment with a molecule inhibiting activity of HMGCR enzyme. Such methods involve measurement of a gene expression signature in the primary melanoma, and determination based at least in part on gene expression levels in that signature that the subject has a high risk of developing metastatic melanoma. A subject determined to have such high risk is selected as a candidate for treatment with a HMGCR inhibitor (a statin).

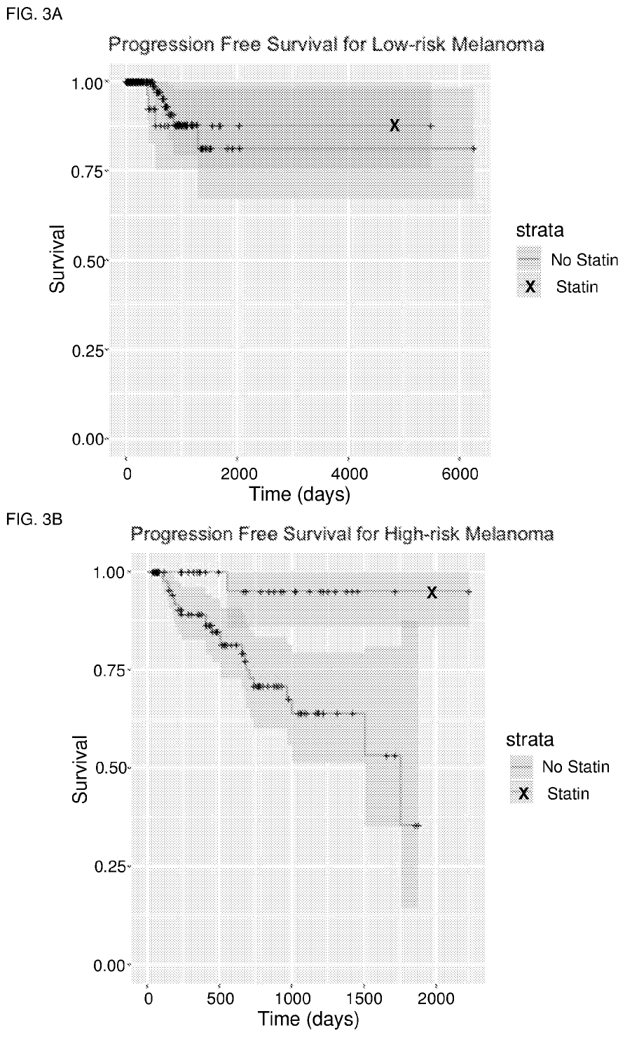

In patients with the low-risk gene expression profile, statin therapy has no effect on 5-year progression free survival (FIG. 3A). However, in patients with a high-risk gene expression profile, statin therapy profoundly improves 5-year progression free survival (FIG. 3B). This data clearly demonstrates the utility of statin therapy in selected patients with high-risk melanoma.

Prior to this disclosure, no evidence existed that demonstrates the utility of statin therapy in genetically selected, high-risk melanoma patients.

There is a clear need in the art for a test which can be used to select melanoma patients who will benefit from preventive treatment to reduce future disease progression, such as metastasis. The present invention involves the use of a gene expression test to determine whether a melanoma patient would benefit from statin therapy to prevent future metastasis and prolong survival. A test designed to select patients for treatment that can prevent future metastasis, as contained in this application, would be a significant advance in the field.

In one embodiment, the invention disclosed herein is a method for selecting patients afflicted by melanoma for treatment with a statin (HMGCR inhibitor), the method involving: measuring (directly or indirectly) the gene expression levels of at least one gene selected from those listed in Table 5, in a sample taken from the primary cutaneous melanoma tumor. In some embodiments, measuring gene-expression levels of the at least one genes includes measurement of a level of fluorescence by a sequence detection system following RT-PCR. Other methods for detecting and measuring the level of expression of a target gene are well known in the art; representative methods are described and/or referenced herein. In addition, indirect methods to determine the level (or change) in gene expression of a target gene are also contemplated. One such indirect method involves measuring the level of methylation of a sequence that is associated with or part of the gene for which expression level is being determined—this is useful, because it is well recognized that there is a correlation between DNA methylation and gene expression (see, e.g., Anastasiadi et al., Epigenetics & Chromatin 11:37, 2018; Tate & Bird, Curr. Op Gen Devel., 3(2):226-231, 1993). Methods of detecting methylation of genomic DNA, including from a biopsy sample, are known (see, e.g., U.S. Pat. Nos. 10,683,551 and 8,088,581).

The method of selecting patients for treatment with a statin further involves determining a gene expression profile of the primary melanoma tumor, involving the gene expression levels of at least one gene; comparing the gene expression profile to the gene expression profile of a reference training set; providing an indication as to whether the primary cutaneous melanoma tumor is low-risk or high-risk of metastasis when the gene expression profile indicates that expression levels of at least one gene is altered in a predictive manner as compared to the gene expression profile of the reference training set; and treating the patient with a statin or other HMGCR inhibitor when the cutaneous melanoma is determined to be at high-risk for metastasis.

One embodiment is a method of reducing risk of future melanoma metastasis and/or progression in a subject with high-risk primary melanoma, which method includes selecting a subject with high-risk primary melanoma, and administering to the selected subject a composition including an inhibitor of 3-Hydroxy-3-Methylglutaryl-CoA Reductase (HMGCR) (a statin, such as fluvastatin, pitavastatin, atorvastatin, simvastatin, lovastatin, rosuvastatin, or pravastatin). In examples of this method, the primary melanoma is determined to be high-risk based on one or more genetic features. By way of example, the genetic features may include: a mutation in any of the genes listed in Table 5; or a change in expression of any of the genes listed in Table 5, compared to expression of the gene in a control or reference sample.

Also contemplated are method embodiments that further include treating the subject with immunotherapy or chemotherapy or both, as well as method embodiments that further include treating the subject with nicotinamide or niacin or both.

Another embodiment is a method of selecting a subject afflicted with primary melanoma for treatment with a statin, the method including measurement of a gene expression profile (GEP) in the primary melanoma. For instance, the GEP in example methods includes at least one of the genes listed in Table 5; at least five of the genes listed in Table 5; at least ten of the genes listed in Table 5; at least twenty six (26) of the genes listed in Table 5. In additional examples, the gene expression profile includes at least 30, at least 40, at least 50, or at least 60, at least 70, at least 80, at least 90, at least 100, at least 125, or more than 120 of the genes listed in Table 5. One specific example includes a method wherein the gene expression profile includes all of the genes ANGPT2, ABCC12, ACOT1, ADAM12, AGBL4, AGXT, AIM2, ALK, ANGPT1, ANGPTL7, ANK1, AQP3, ARG1, ARRDC1, ART1, BAP1, BIN1, BMP2, BMX, BTG1, C8G, CACNG4, CAMK2B, CASQ1, CCR3, CCR5, CDC5L, CENPQ, CETP, CLCA2, CLIC5, COL24A1, CPN2, CRABP2, CST6, CTAGE1, CTCFL, CUL7, CXCL14, CXCL8/IL8, DIO3OS, DMAP1, DOCK3, DPEP3, DSC1, DYSF, EEF1A2, EHBP1Li, EIF1B, ERGIC2, F7, FASLG, FGF2, FLOT1, FLVCR2, FNBP1L, FREM2, GABBR2, GBP5, GCH1, GDF15, GFRA1, GJA1, GLDC, GPR39, GPR63, GRAMD1B, HEPACAM, HHATL, HMGCR, HNF4A, HTR3B, ID2, IFNG, ITGB3, JARID2, JPH3, KCNT1, KIF19, KLC4, KLHDC8A, KNDC1, KRT14, KRT6B, KRTAP19-6, KSR2, KYNU, LAG3, LAMB1, LGALSI, LGI4, LRRC31, LTA4H, LOXL4, MCF2, ME3, MFSD6L, MGP, MIR222, MLANA, MLF1, MTUS2, NFYA, NPPC, NTRK1, NXPH1, OCIAD2, OR52K1, OR5AK2, P2RY14, PARP11, PHEX, PIK3R6, PLA2G2D, PLAT, PLCZ1, PPL, PRF1, PRKCB, PROCA1, PTPRG, RAB15, RBBP4, RBM23, RGS7, RNF213, ROBO1, RP1, RPL7L1, S100A8, S100A9, S1PR1, SAP130, SERPINE2, SFMBT2, SIGLEC12, SLC17A3, SLC29A1, SLCO5A1, SLIT1, SOCS1, SOX4, SPAG6, SPP1, SPRR1B, STMN1, TACSTD2, TAS2R60, TDRD12, TFF2, TGFB1, TGFBRI, TLR4, TMCC3, TNFRSF10A, TP53, TRIM22, TRIM29, TRNAU1AP, TTN, TUBB, TYRP1, UTS2, VAMP5, VEGFA, VEGFC, VEGFD, WDR49, WIF1, ZIM2, ZNF560, and ZNF697.

Yet another provided embodiment is a method of selecting a subject afflicted with melanoma for treatment with a molecule inhibiting activity of HMGCR enzyme, the method including measurement of a gene expression signature in the primary melanoma.

In examples of any of the above embodiments, the subject afflicted with primary melanoma is selected for treatment with a statin in order to reduce likelihood of a future metastasis of the melanoma.

Also provided is a method of selecting a melanoma patient for treatment with a statin (HMGCR inhibitor), the method including: obtaining a sample from a primary cutaneous melanoma tumor in the subject; measuring gene expression levels of at least two genes listed in Table 5 in the sample; producing a gene expression profile including the gene expression levels of the at least two genes; comparing the gene expression profile to a gene expression profile of a reference training set gene expression profile, which training set gene expression profile includes the same at least two genes; and generating an indication that the primary cutaneous melanoma tumor is low-risk or high-risk of metastasis when the gene expression profile indicates that expression levels of at least one gene is altered in a predictive manner as compared to the gene expression profile of the reference training set By way of example, in embodiments of this method, measuring gene expression levels includes measurement of a level of fluorescence by a sequence detection system following RT-PCR. Optionally, in these methods of selecting a melanoma patient for treatment with a statin further includes treating the patient with a statin or other HMGCR inhibitor when the primary cutaneous melanoma is determined to be at high-risk for metastasis.

This disclosure also provides a method of treating melanoma in a patient having a primary melanoma tumor, the method including: administering to the patient a therapeutically effective dose of an inhibitor of 3-Hydroxy-3-Methylglutaryl-CoA Reductase (HMGCR) (a statin, such as fluvastatin, pitavastatin, atorvastatin, simvastatin, lovastatin, rosuvastatin, or pravastatin). In examples, the primary melanoma tumor is a Stage 3 (regional) or Stage 4 (metastatic) melanoma.

Yet another embodiment is a method of treating melanoma in a patient having a primary melanoma tumor, the method including administering to the patient: a therapeutically effective dose of an inhibitor of 3-Hydroxy-3-Methylglutaryl-CoA Reductase (HMGCR) (a statin, such as fluvastatin, pitavastatin, atorvastatin, simvastatin, lovastatin, rosuvastatin, or pravastatin); and a therapeutically effective dose of a melanoma treatment selected from a BRAF inhibitor and an immunotherapy for treatment of melanoma. In examples of this method embodiment, the statin and the melanoma treatment are administered concurrently, or sequentially. By way of example, the BRAF inhibitor may be any recognized inhibitor of BRAF, such as Vemurafenib, dabrafenib, GDC-0879, PLX 4032, PLX-4720, PLX 4734, Sorafenib Tosylate, or a combination of trametinib and dabrafenib.

In each method embodiment provided herein, examples are included in which the subject is not treated with a RORgamma inhibitor.

Aspects of the current disclosure are now described with additional details and options as follows: (I) Melanoma Identification and Staging; (II) Representative Definitions of Terms; (Ill) Statin Treatments; (IV) Pharmaceutical Compositions and Administration Formulations; (V) Gene Expression Profile/Profiling; (VI) Melanoma Treatment including Combination Therapies; (VII) Exemplary Embodiments; (VIII) Experimental Examples; and (IX) Closing Paragraphs. These headings do not limit the interpretation of the disclosure and are provided for organizational purposes only.

### 1. Melanoma Detection and Staging

Visual diagnosis of melanomas is the most common method employed by health professionals. Moles that are irregular in color or shape are often treated as candidates of melanoma. The diagnosis of melanoma requires experience, as early stages may look identical to harmless moles or not have any color at all. People with a personal or family history of skin cancer or of dysplastic nevus syndrome (multiple atypical moles) should see a dermatologist at least once a year to be sure they are not developing melanoma. Metastatic melanomas have been detected by X-rays, CT scans, MRIs, PET and PET/CTs, ultrasound, LDH testing, and photoacoustic detection.

Many melanomas present themselves as lesions smaller than 6 mm in diameter; and all melanomas were malignant on day 1 of growth, which is merely a dot. An astute physician will examine all abnormal moles, including ones less than 6 mm in diameter. Seborrheic keratosis may meet some or all of the identification criteria, and can lead to false alarms among laypeople and sometimes even physicians. An experienced doctor can generally distinguish seborrheic keratosis from melanoma upon examination, or with dermatoscopy.

Total body photography, which involves photographic documentation of as much body surface as possible, is often used during follow-up of high-risk patients. The technique has been reported to enable early detection and provides a cost-effective approach (being possible with the use of any digital camera), but its efficacy has been questioned due to its inability to detect macroscopic changes. The diagnosis method should be used in conjunction with (and not as a replacement for) dermatoscopic imaging, with a combination of both methods appearing to give extremely high rates of detection.

Melanoma is divided into the following types: Lentigo maligna melanoma, Superficial spreading melanoma, Acral lentiginous melanoma, Mucosal melanoma, Nodular melanoma, Polypoid melanoma, Desmoplastic melanoma, Amelanotic melanoma, Soft-tissue melanoma, Melanoma with small nevus-like cells, Melanoma with features of a Spitz nevus Uveal melanoma. Confirmation of a clinical diagnosis is achieved with a skin biopsy. This is usually followed up with a wider excision of the scar or tumor.

Melanoma stages (see below) depend on the thickness of the tumor, whether cancer has spread to lymph nodes or other parts of the body, and other factors (such as ulceration, Depending on the stage, a sentinel lymph node biopsy is done.

| | | 5 year |
|---|---|---|
| Stage | Description/Subtypes | survival rate |
| 0 | Melanoma in situ (Clark Level I) | 99.9% |
| I/II | Invasive melanoma | 85-99% |
| | T1a: Less than 1.00 mm primary tumor thickness, without | |
| | ulceration, and mitosis <1/mm² | |
| | T1b: Less than 1.00 mm primary tumor thickness, with ulceration | |
| | or mitoses ≥1/mm^{²} | |
| | T2a: 1.00-2.00 mm primary tumor thickness, without ulceration | |
| II | High risk melanoma | 40-85% |
| | T2b: 1.00-2.00 mm primary tumor thickness, with ulceration | |
| | T3a: 2.00-4.00 mm primary tumor thickness, without ulceration | |
| | T3b: 2.00-4.00 mm primary tumor thickness, with ulceration | |
| | T4a: 4.00 mm or greater primary tumor thickness without | |
| | ulceration | |
| | T4b: 4.00 mm or greater primary tumor thickness with ulceration | |
| III | Regional metastasis | 25-60% |
| | N1: Single positive lymph node | |
| | N2: Two to three positive lymph nodes or regional skin/in-transit | |
| | metastasis | |
| | N3: Four positive lymph nodes or one lymph node and regional | |
| | skin/in-transit metastases | |
| IV | Distant metastasis | 9-15% |
| | M1a: Distant skin metastasis, normal LDH | |
| | M1b: Lung metastasis, normal LDH | |
| | M1c: Other distant metastasis or any distant metastasis with | |
| | elevated LDH | |

### II. Representative Definitions of Terms

As used herein, the phrase “altered in a predictive manner” means changes in genetic expression profile that predict metastatic risk.

As used herein, “metastasis” is defined as the recurrence or disease progression that may occur locally (such as local recurrence and in transit disease), regionally (such as nodal micrometastasis or macro metastasis), or distally (such as brain, lung and other tissues). Metastases refers to the process through which a tumor in a primary site releases single tumor cells that seed other distant organ sites. This phase of tumorigenesis is most fatal (90% mortality). Metastatic disease or metastasis refers to cancer cells that have left the original tumor site and migrate to other parts of the body for example via the bloodstream or lymph system. The “pathology” of cancer includes all phenomena that compromise the well-being of the subject. This includes, abnormal or uncontrollable cell growth, metastasis, interference with the normal functioning of neighboring cells, release of cytokines or other secretory products at abnormal levels, suppression or aggravation of inflammatory or immunological response, neoplasia, premalignancy, malignancy, invasion of surrounding or distant tissues or organs, such as lymph nodes, etc.

The phrase “sequence detection system” refers to any computation method that is used to analyze the results of a PCR or other nucleic acid amplification reaction. Gene expression may be analyzed by direct DNA expression in microarray, Sanger sequencing analysis, Northern blot, Nanostring® technology, serial analysis of gene expression (SAGE), RNA-seq, tissue microarray, or protein expression with immunohistochemistry or western blot technique.

A “reference training set” as the phrase is used herein is a clinical cohort of cutaneous melanoma tumors with known metastatic outcome and known genetic expression profile used as a reference to compare other cutaneous melanomas and assign them as high or low risk for metastasis. Analysis of genetic expression and comparison to this reference set may be accomplished by any computational method in the art radial basis machine and/or partition tree analysis, LRA, K-nearest neighbor, or other algorithmic approaches.

As the phrase is used herein, a “high-risk melanoma” is defined by a characteristic gene expression pattern (or profile). This gene expression signature may consist of any combination of the genes listed in Table 5. For instance, in embodiments the gene expression panel includes of the genes in Table 1. In another embodiment, the gene expression panel includes the genes in Table 2. In additional embodiments, the gene expression panel includes the genes in Table 3. In additional embodiments, the gene expression panel includes the genes in Table 4. The direction of gene expression (decreased or increased) that would predict a melanoma is high risk is noted in Tables 2-4. The assignment of “high-risk” may be made by comparing the gene expression level of the gene(s) in the melanoma in question to the level of the gene(s) in a reference training set of melanomas. The directionality of gene expression change (increased or decreased) that indicates high-risk for melanoma metastasis is provided.

The term subject (interchangeable with “individual) is intended to mean a living multicellular vertebrate organism, a category that includes, for example, mammals and birds. A mammal includes human as well as non-human mammals, such as mice. In some instances, a subject is a patient, such as a patient diagnosed with melanoma. In other examples, a subject is a patient yet to be diagnosed.

### III. Statin Treatments

As described herein, it has been discovered that a history of statin treatment can impact or influence the lymph node status, disease progression, likelihood of metastasis, and/or overall prognosis of a subject with primary melanoma. Further, it is demonstrated that treatment with a statin can reduce the risk of melanoma progression to metastasis, that statins can be used to treat State 3 or Stage 4 melanoma (even without needing to screen a subject first by GEP), and that statin treatment in combination with other anti-cancer therapy (such as treatment with a BRAF inhibitor or immunotherapy) improves patient outcomes such as survival.

In general, the statin treatment contemplated is conventional for therapeutic inhibition of 3-hydroxy-3-methyl-glutaryl-coenzyme A reductase (HMGCoA reductase).

The statin compounds administered to a subject in need thereof (such as a subject diagnosed with melanoma, or at risk for melanoma progression or melanoma metastasis) may be administered in any appropriate fashion, for instance directed by a medical professional based upon the individual subject's age, weight, medical condition, and other medications or treatments received prior to or concurrent with the statin administrations in question. The subject may receive the statin in question through any route of administration determined by a medical professional. In some embodiments, the statin in question is administered orally.

For instance, the statin is administered to the subject, for instance a human subject or patient, in a “therapeutically effective amount” or “pharmaceutically effective amount,” which refers to an amount that is sufficient to effect treatment, as described herein, when administered to a subject in need of such treatment.

The terms “treat,” “treatment” or “treating,” as used herein, refer to an approach for obtaining beneficial or desired results including clinical results. Beneficial or desired clinical results may include one or more of the following: (i) inhibiting the disease or condition (e.g., decreasing one or more symptoms resulting from the disease or condition, and/or diminishing the extent of the disease or condition); (ii) slowing or arresting the development of one or more clinical symptoms associated with the disease or condition (e.g., stabilizing the disease or condition, preventing or delaying the worsening or progression of the disease or condition, and/or preventing or delaying the spread (e.g., metastasis) of the disease or condition); and/or (iii) relieving the disease, that is, causing the regression of clinical symptoms (e.g., ameliorating the disease state, providing partial or total remission of the disease or condition, enhancing effect of another medication, delaying the progression of the disease, increasing the quality of life, and/or prolonging survival).

Based on the discoveries described herein, it is also contemplated that a “beneficial or desired clinical result” may be felt in a subject for a disease or condition that is different from the one for which the statin is or was primarily prescribed. Thus, a subject who is (or was) prescribed a statin in order to lower cholesterol and/or protect against heart attack and/or stroke may also receive from the same statin a beneficial and desirable clinical result including one or more of lowered risk of melanoma metastasis, improved melanoma prognosis, reduced likelihood of spread of the melanoma to a lymph node, lower likelihood of positive SLNB, and so forth.

Fluvastatin, available commercially under the tradenames LESCOL® and LESCOL XL®, may be administered to a subject in need thereof at a daily dose of from 1 mg to 100 mg. In separate embodiments, the fluvastatin may be administered to the subject at, respectively, 1 mg/day, 5 mg/day, 10 mg/day, 20 mg/day, 25 mg/day, 30 mg/day, 40 mg/day, 50 mg/day, 60 mg/day, 70 mg/day, 80 mg/day, 90 mg/day, and 100 mg/day.

Pitavastatin, commercially available under the LIVALO® tradename, may be administered to a subject in need thereof at a daily dose of from 0.1 mg to 10 mg. In some embodiments, the pitavastatin may be administered to the subject in need thereof at a daily dose of from 0.1 mg to 5 mg per day. In other embodiments, pitavastatin may be administered at a dose of from 1 mg/day to 5 mg/day. Individual doses in separate embodiments may be selected from the group of 1 mg/day, 1.5 mg/day, 2 mg/day, 2.5 mg/day, 3 mg/day, 3.5 mg/day, 4 mg/day, 4.5 mg/day, and 5 mg/day.

Atorvastatin, commercially available as atorvastatin calcium under the LIPITOR® tradename, may be administered to a subject in need thereof at a daily dose of from 1 mg to 100 mg. In separate embodiments, the atorvastatin may be administered to the subject at, respectively, 5 mg/day, 10 mg/day, 20 mg/day, 25 mg/day, 30 mg/day, 40 mg/day, 50 mg/day, 60 mg/day, 70 mg/day, 80 mg/day, 90 mg/day, and 100 mg/day.

Simvastatin, commercially available under the ZOCOR® tradename, may be administered to a subject in need thereof at a daily dose of from 1 mg to 100 mg. In separate embodiments, the simvastatin may be administered to the subject at, respectively, 5 mg/day, 10 mg/day, 20 mg/day, 25 mg/day, 30 mg/day, 40 mg/day, 50 mg/day, 60 mg/day, 70 mg/day, 80 mg/day, 90 mg/day, and 100 mg/day.

Lovastatin, commercially available under the MEVACOR® AND ALSOPREV® tradenames, may be administered to a subject in need thereof at a daily dose of from 1 mg to 100 mg. In separate embodiments, the lovastatin is administered to the subject at, respectively, 5 mg/day, 10 mg/day, 20 mg/day, 25 mg/day, 30 mg/day, 40 mg/day, 50 mg/day, 60 mg/day, 70 mg/day, 80 mg/day, 90 mg/day, and 100 mg/day.

Rosuvastatin, commercially available under the CRESTOR® AND EZALLOR SPRINKLE© tradenames, may be administered to a subject in need at a daily dose of from 1 mg to 100 mg. In separate embodiments, the rosuvastatin may be administered to the subject at, respectively, 5 mg/day, 10 mg/day, 20 mg/day, 25 mg/day, 30 mg/day, 40 mg/day, 50 mg/day, 60 mg/day, 70 mg/day, 80 mg/day, 90 mg/day, and 100 mg/day.

Pravastatin, commercially available under the PRAVACHOL® tradename, may be administered to a subject in need thereof at a daily dose of from 1 mg to 100 mg. In separate embodiments, the pravastatin may be administered to the subject at, respectively, 5 mg/day, 10 mg/day, 20 mg/day, 25 mg/day, 30 mg/day, 40 mg/day, 50 mg/day.

Although exemplified herein with subjects who were taking a statin at the time of biopsy of their primary melanoma tumor, it is also believed that prior statin treatment that has ceased before a biopsy is taken (or before melanoma is diagnosed) would influence (at least for a time) the subject's gene expression. Thus, statin-related effect(s) continue for a period of time after the subject has stopped taking the statin. It is not required that the subject be actively taking a statin at the time their melanoma is identified or diagnosed, or at the time a tissue biopsy is taken, an analysis of their disease state is carried out, at prognosis, or when any other melanoma-based analysis takes place. Broadly, therefore, the phrase “a subject with history of taking a statin” is intended to include a subject who is currently taking a statin and has done so for at least a week or more, as well as a subject who was taking a statin for a period of time but who stopped doing so before the selected time point. By way of example, the subject may have taken a statin until a day before the selected time point, or a week before, or two weeks before, or a month before, or two months before, or longer. In exemplary embodiments, a “subject with a history of taking a statin” is one who took statin(s) for at least six months, or at least a year, and is still taking a stating at the time of diagnosis with melanoma.

The “history of taking a statin” status of an individual can be determined in any conventional way, including: review of the individual's medical history or files; asking the individual; consulting with a physician or other medical professional who has treated or is treating the individual (for instance, a medical professional who is treating the individual for their general health, or their cardiac or cardiovascular health, even if that professional is not treating the individual for melanoma); and analyzing blood or another sample from the individual for alterations caused by having taken statins (e.g., changes in cholesterol and/or triglycerides), or the presence of a statin or statin degradation product.

### IV. Pharmaceutical Compositions and Administration Formulations

Provided herein are methods and compositions for treatment, for instance treatment of cancer, specifically treatment of melanoma, metastatic melanoma, or related conditions. Appropriate active/therapeutic compounds for such treatments are discussed herein, and additional appropriate active compounds are known to those of ordinary skill in the art.

When formulated in a pharmaceutical composition, a therapeutic compound can be admixed with a pharmaceutically acceptable carrier or excipient. As used herein, the phrase “pharmaceutically acceptable” refers to molecular entities and compositions that are generally believed to be physiologically tolerable and do not typically produce an allergic or similar untoward reaction, such as gastric upset, dizziness and the like, when administered to a human or veterinary subject.

The term “pharmaceutically acceptable derivative” as used herein means any pharmaceutically acceptable salt, solvate or prodrug, e.g. ester, of the desired active agent, which upon administration to the recipient is capable of providing (directly or indirectly) the desired active agent, or an active metabolite or residue thereof. Such derivatives are recognizable to those skilled in the art, without undue experimentation. Nevertheless, reference is made to the teaching of Burger's Medicinal Chemistry and Drug Discovery, 5th Edition, Vol 1: Principles and Practice. Pharmaceutically acceptable derivatives include salts, solvates, esters, carbamates, and phosphate esters.

While it is possible to use a composition for therapy as is, it may be preferable to administer it in a pharmaceutical formulation, e.g., in admixture with a suitable pharmaceutical excipient, diluent or carrier selected with regard to the intended route of administration and standard pharmaceutical practice. Accordingly, in one aspect, pharmaceutical composition or formulation includes at least one active composition, or a pharmaceutically acceptable derivative thereof, in association with a pharmaceutically acceptable excipient, diluent and/or carrier. The excipient, diluent and/or carrier is “acceptable” in the sense of being compatible with the other ingredient(s) of the formulation and not significantly deleterious to the recipient thereof.

Any composition formulation disclosed herein can advantageously include any other pharmaceutically acceptable carriers which include those that do not produce significantly adverse, allergic, or other untoward reactions that outweigh the benefit of administration, whether for research, prophylactic and/or therapeutic treatments. Exemplary pharmaceutically acceptable excipients, diluents, and carriers for therapeutic use are well known in the pharmaceutical art, and are described, for example, in Remington: The Science and Practice of Pharmacy. Lippincott Williams & Wilkins (A.R. Gennaro edit. 2005), and in n Remington's Pharmaceutical Sciences, 18th Ed. Mack Printing Company, 1990. Moreover, formulations can be prepared to meet sterility, pyrogenicity, general safety and purity standards as required by United States FDA Office of Biological Standards and/or other relevant foreign regulatory agencies. The pharmaceutical excipient(s), diluent(s), and carrier(s) can be selected with regard to the intended route of administration and standard pharmaceutical practice.

Such pharmaceutical formulations may be presented for use in a conventional manner with the aid of one or more suitable excipients, diluents, and carriers. Pharmaceutically acceptable excipients assist or make possible the formation of a dosage form for a bioactive material and include diluents, binding agents, lubricants, glidants, disintegrants, coloring agents, and other ingredients. Preservatives, stabilizers, dyes and even flavoring agents may be provided in the pharmaceutical composition. Examples of preservatives include sodium benzoate, ascorbic acid and esters of p-hydroxybenzoic acid. Antioxidants and suspending agents may be also used. An excipient is pharmaceutically acceptable if, in addition to performing its desired function, it is non-toxic, well tolerated upon ingestion, and does not interfere with absorption of bioactive materials.

Exemplary generally used pharmaceutically acceptable carriers include any and all bulking agents or fillers, solvents or co-solvents, dispersion media, coatings, surfactants, antioxidants (e.g., ascorbic acid, methionine, vitamin E), preservatives, isotonic agents, absorption delaying agents, salts, stabilizers, buffering agents, chelating agents (e.g., EDTA), gels, binders, disintegration agents, and/or lubricants.

Exemplary buffering agents include citrate buffers, succinate buffers, tartrate buffers, fumarate buffers, gluconate buffers, oxalate buffers, lactate buffers, acetate buffers, phosphate buffers, histidine buffers and/or trimethylamine salts.

Exemplary preservatives include phenol, benzyl alcohol, meta-cresol, methyl paraben, propyl paraben, octadecyldimethylbenzyl ammonium chloride, benzalkonium halides, hexamethonium chloride, alkyl parabens such as methyl or propyl paraben, catechol, resorcinol, cyclohexanol and 3-pentanol.

Exemplary isotonic agents include polyhydric sugar alcohols including trihydric or higher sugar alcohols, such as glycerin, erythritol, arabitol, xylitol, sorbitol, or mannitol.

Exemplary stabilizers include organic sugars, polyhydric sugar alcohols, polyethylene glycol; sulfur-containing reducing agents, amino acids, low molecular weight polypeptides, proteins, immunoglobulins, hydrophilic polymers, or polysaccharides.

The pharmaceutical compositions can be formulated for administration in any convenient way for use in human or veterinary medicine. Exemplarily modes of administration are discussed herein.

A therapeutically effective amount means the amount of a compound that, when administered to an animal subject or treating a state, disorder or condition, is sufficient to effect such state, disorder, or condition. The therapeutically effective amount will vary depending on the compound, the disease and its severity and the age, weight, physical condition and responsiveness of the mammal to be treated. In certain cases, the phrase therapeutically effective amount is used to mean an amount or dose sufficient to modulate, e.g., increase or decrease a desired activity e.g., by 10 percent, by 50 percent, or by 90 percent. Generally, a therapeutically effective amount is sufficient to cause an improvement in a clinically significant condition in the subject following a therapeutic regimen involving one or more therapeutic agents. The concentration or amount of the active ingredient depends on the desired dosage and administration regimen, as discussed below. Suitable dosages may range from 0.01 mg/kg to 100 mg/kg of body weight per day, week, or month.

The actual dose amount administered to a particular subject can be determined by a physician, veterinarian, or researcher taking into account parameters such as physical, physiological and psychological factors including target, body weight, stage of cancer, the type of cancer, previous or concurrent therapeutic interventions, idiopathy of the subject, and route of administration.

Exemplary doses can include 0.05 mg/kg to 5.0 mg/kg of the active compounds (drugs) disclosed herein. The total daily dose can be 0.05 mg/kg to 30.0 mg/kg of an agent administered to a subject one to three times a day, including administration of total daily doses of 0.05-3.0, 0.1-3.0, 0.5-3.0, 1.0-3.0, 1.5-3.0, 2.0-3.0, 2.5-3.0, and 0.5-3.0 mg/kg/day of administration forms of a drug using 60-minute oral, intravenous or other dosing. In one particular example, doses can be administered QD or BID to a subject with, e.g., total daily doses of 1.5 mg/kg, 3.0 mg/kg, or 4.0 mg/kg of a composition with up to 92-98% wt/v of the compounds disclosed herein. Additional useful doses can often range from 0.1 to 5 μg/kg or from 0.5 to 1 μg/kg. In other examples, a dose can include 1 μg/kg, 10 μg/kg, 20 μg/kg, 40 μg/kg, 80 μg/kg, 200 μg/kg, 0.1 to 5 mg/kg or from 0.5 to 1 mg/kg. In other examples, a dose can include 1 mg/kg, 10 mg/kg, 20 mg/kg, 40 mg/kg, 80 mg/kg, 200 mg/kg, 400 mg/kg, 450 mg/kg, or more. Additional specific dosages are described herein for specific drugs.

Therapeutically effective amounts can be achieved by administering single or multiple doses during the course of a treatment regimen (e.g., hourly, every 2 hours, every 3 hours, every 4 hours, every 6 hours, every 9 hours, every 12 hours, every 18 hours, daily, every other day, every 3 days, every 4 days, every 5 days, every 6 days, weekly, every 2 weeks, every 3 weeks, or monthly).

A therapeutically effective amount of the desired active agent can be formulated in a pharmaceutical composition to be introduced parenterally, transmucosally (e.g., orally, nasally, or rectally), or transdermally. In some embodiments, administration is parenteral, for instance, via intravenous injection, or intra-arteriole, intramuscular, intradermal, subcutaneous, intraperitoneal, intraventricular, and intracranial administration.

In another embodiment, the active ingredient can. be delivered in a vesicle, in particular a liposome (see Langer, Science, 1990; 249:1527-1533; Treat et al, in Liposomes in the Therapy of Infectious Disease and Cancer, Lopez-Berestein and Fidler (eds.), Liss: New York, pp. 353-365 (1989); Lopez-Berestein, ibid., pp. 317-327).

The effective amounts of compounds containing active agents include doses that partially or completely achieve the desired therapeutic, prophylactic, and/or biological effect. The actual amount effective for a particular application depends on the condition being treated and the route of administration. The effective amount for use in humans can be determined from animal models. For example, a dose for humans can be formulated to achieve circulating and/or gastrointestinal concentrations that have been found to be effective in animals.

For administration, therapeutically effective amounts (also referred to herein as doses) can be initially estimated based on results from in vitro assays and/or animal model studies. Such information can be used to more accurately determine useful doses in subjects of interest. Particularly useful pre-clinical tests include measure of cell growth, cell death, and/or cell viability. In particular, measurement of (T) cell exhaustion may be beneficial.

The pharmaceutical compositions may also include other biologically active compounds.

Compositions can also be administered with anesthetics including ethanol, bupivacaine, chloroprocaine, levobupivacaine, lidocaine, mepivacaine, procaine, ropivacaine, tetracaine, desflurane, isoflurane, ketamine, propofol, sevoflurane, codeine, fentanyl, hydromorphone, marcaine, meperidine, methadone, morphine, oxycodone, remifentanil, sufentanil, butorphanol, nalbuphine, tramadol, benzocaine, dibucaine, ethyl chloride, xylocaine, and/or phenazopyridine.

In particular embodiments, the compositions disclosed herein can be used in conjunction with other cancer treatments, such as chemotherapeutic agents, radiation therapy, and/or immunotherapy. The compositions described herein can be administered (except as discussed regarding checkpoint inhibition therapy, which is administered subsequent to cessation of the preconditioning) simultaneously with or sequentially with another treatment within a selected time window, such as within 10 minutes, 1 hour, 3 hour, 10 hour, 15 hour, 24 hour, or 48 hour time windows or when the complementary treatment is within a clinically-relevant therapeutic window.

The compositions described herein can be administered by, a variety of routes.

For injection, compositions can be made as aqueous solutions, such as in buffers such as Hanks' solution, Ringer's solution, or physiological saline. The solutions can contain formulatory agents such as suspending, stabilizing and/or dispersing agents. Alternatively, the composition can be in lyophilized and/or powder form for constitution with a suitable vehicle, e.g., sterile pyrogen-free water, before use.

Compositions can also be formulated for oral administration. For ingestion, compositions can take the form of tablets, pills, lozenges, sprays, liquids, and capsules formulated in conventional manners. Ingestible compositions can be prepared using conventional methods and materials known in the pharmaceutical art. For example, U.S. Pat. Nos. 5,215,754 and 4,374,082 relate to methods for preparing swallowable compositions. U.S. Pat. No. 6,495,177 relates to methods to prepare chewable supplements with improved mouthfeel. U.S. Pat. No. 5,965,162, relates to compositions and methods for preparing comestible units which disintegrate quickly in the mouth.

Ingestible compositions may have a shape containing no sharp edges and a smooth, uniform and substantially bubble free outer coating. Coatings of ingestible compositions can be derived from a polymeric film. Such film coatings reduce the adhesion of the compositions to the inner surface of the mouth and can aid in masking potential unpleasant tastes. Coatings can also protect the compositions from atmospheric degradation. Exemplary polymeric films include vinyl polymers, cellulosics, acrylates and methacrylates, natural gums and resins such as zein, gelatin, shellac and acacia. Other common excipients used in ingestible compositions include sucrose, fructose, lactose, glucose, lycasin, xylitol, lactitol, erythritol, mannitol, isomaltose, dextrose, polydextrose, dextrin, compressible cellulose, compressible honey, compressible molasses, fondant or gums, vegetable oils, animal oils, alkyl polysiloxanes, corn starch, potato starch, pre-gelatinized starches, stearic acid, calcium stearate, magnesium stearate, zinc stearate, benzoic acid, and colorants.

For administration by inhalation (e.g., nasal or pulmonary), the compositions can be formulated as aerosol sprays for pressurized packs or a nebulizer, with the use of suitable propellants, e.g. dichlorodifluoromethane, trichlorofluoromethane, or dichlorotetra-fluoroethane.

### V. Gene Expression Profile/Profiling

In various embodiments, determination of whether a subject has a history of taking a statin is combined with the results from a gene expression profile (GEP) from the subject's primary melanoma tumor, in order to carry out an analysis or provide a classification of the subject.

There are art-recognized systems and methods, useful with the current disclosure, for obtaining cancer tissue samples (e.g., tissue form a primary melanoma tumor, such as a primary cutaneous melanoma), analyzing such samples for the expression level of target gene(s), and/or the methylation level of target gene(s) (as a stand-in or indirect measure of gene expression levels, or a supplement thereto), assembling or producing gene expression profile(s) that contain or include the level of expression (or of methylation) of two or more genes expressed from the melanoma tissue sample, preparing reference sample sets and reference set gene expression profiles, and related methods. See, for instance, U.S. Pat. Nos. 9,410,205, 10,577,660 and 10,233,502; U.S. Application Publication No. US20200362419A1 and International Patent Publication WO2020022895A2. Representative methods are also described herein.

Genetic expression can refer to whether a cell (i) has a particular sequence of a gene, and/or (ii) whether the cell is expressing the gene and/or (iii) whether the protein produced is maintained/stable in the cell or system. Through the process of transcription, a cell expressing a gene generates RNA sequences corresponding to that gene. Accordingly, in various embodiments, expression of a particular gene from a tumor is identified based on the presence and/or amount of RNA sequence(s) in the tumor or a sample from the tumor. Particular embodiments of the present disclosure include identifying the presence and/or amount of particular RNA sequence(s) corresponding to the set of genetic biomarkers. Various methods and systems described herein involve RNA (transcriptome) analysis. It will be understood that the isolation, detection, and quantification of RNA, including specific RNA corresponding to a specific target gene (such as any or all of the genes that are included in a detection model provided herein), can be carried out using any art-recognized methods. These include for instance, array-based detection methods as well as sequencing.

Methods for analyzing gene expression include methods based on hybridization analysis of polynucleotides, sequencing of polynucleotides, and analysis of protein expression (e.g., proteomics-based methods). Commonly used methods for the quantification of mRNA expression in a sample include northern blotting and in situ hybridization (Parker & Barnes, Meth Mol Biol 106:247-283, 1999); RNAse protection assays (Hod, Biotechniques 13:852 854, 1992); and PCR-based methods, such as reverse transcription polymerase chain reaction (RT-PCR) (Weis et al., Trends in Genetics 8:263 264, 1992). Alternatively, antibodies may be employed that can recognize specific duplexes, including DNA duplexes, RNA duplexes, and DNA-RNA hybrid duplexes or DNA-protein duplexes. Representative methods for sequencing-based gene expression analysis include Serial Analysis of Gene Expression (SAGE), and gene expression analysis by massively parallel signature sequencing (MPSS).

Evaluating gene expression of a melanoma sample can be performed with microarrays. Microarrays permit simultaneous analysis of a large number of gene expression products. Typically, polynucleotides of interest are plated, or arrayed, on a microchip substrate. The arrayed sequences are then hybridized with nucleic acids (e.g., DNA or RNA) from cells or tissues of interest (e.g., cutaneous tissue samples). The source of mRNA typically is total RNA (e.g., total RNA isolated from human melanoma samples, and normal skin samples). If the source of mRNA is a primary tumor, mRNA can be extracted, for example, from frozen or archived paraffin-embedded and fixed (e.g. formalin-fixed) tissue samples.

In various embodiments of the microarray technique, probes to (e.g., specific for) at least 2, 6, 10, 25, 50, 100, 150, 200, or more genes are immobilized on an array substrate (e.g., a porous or nonporous solid support, such as a glass, plastic, or gel surface). The probes can include DNA, RNA, copolymer sequences of DNA and RNA, DNA and/or RNA analogues, or combinations thereof.

In some embodiments, a microarray includes a support with an ordered array of binding (e.g., hybridization) sites for each individual gene. The microarrays can be addressable arrays, for instance positionally addressable arrays, i.e., each probe of the array is located at a known, predetermined position on the solid support such that the identity (i.e., the sequence) of each probe can be determined from its position in the array.

Each probe on the microarray can be between 10-50,000 nucleotides, e.g., between 300-1,000 nucleotides, in length. The probes of the microarray can consist of nucleotide sequences with lengths: less than 1,000 nucleotides, e.g., sequences 10-1,000, or 10-500, or 10-200 nucleotides in length. An array can include positive control probes, e.g., probes known to be complementary and hybridizable to sequences in the test sample; and negative control probes, e.g., probes known to not be complementary and hybridizable to sequences in the test sample.

Methods for attaching nucleic acids to a surface are known. Methods for immobilizing nucleic acids on glass have been described (Schena et al, Science 270:467-470, 1995; DeRisi et al, Nature Genetics 14:457-460, 1996; Shalon et al., Genome Res. 6:639-645, 1996; and Schena et al., Proc. Natl. Acad. Sci. U.S.A. 93:10539-11286, 1995). Techniques are known for producing arrays with thousands of oligonucleotides at defined locations using photolithographic techniques are described by Fodor et al. (Science 251:767-773, 1991), Pease et al. (Proc. Natl. Acad. Sci. U.S.A. 91:5022-5026, 1994), Lockhart et al. (Nature Biotechnology 14:1675, 1996), and in U.S. Pat. Nos. 5,578,832; 5,556,752; and 5,510,270. Other methods for making microarrays have been described. See, e.g., Maskos and Southern, Nuc. Acids. Res. 20:1679-1, 684, 1992. In principle, any type of array, for example, dot blots on a nylon hybridization membrane (see Sambrook et al., Molecular Cloning, A Laboratory Manual, 2^{nd} Ed., Vols. 1-3, Cold Spring Harbor Laboratory, Cold Spring Harbor, N.Y. (1989)) could be used.

The polynucleotide molecules to be analyzed using a microarray may be from any clinically relevant source (such as from a portion of a melanoma tissue biopsy), and are expressed RNA or a nucleic acid derived therefrom (e.g., cDNA or amplified RNA derived from cDNA that incorporates an RNA polymerase promoter), including naturally occurring nucleic acid molecules, as well as synthetic nucleic acid molecules. For example, the test polynucleotide molecules include total cellular RNA, poly(A)+ messenger RNA (mRNA), or fraction thereof, cytoplasmic mRNA, or RNA transcribed from cDNA (i.e., cRNA). Methods for preparing RNA are known and are described, e.g., in Sambrook et al., Molecular Cloning, A Laboratory Manual (2Supnd/SupEd.), Vols. 1-3, Cold Spring Harbor Laboratory, Cold Spring Harbor, N.Y., 1989. RNA can be fragmented by methods known in the art, e.g., by incubation with ZnCl₂, to generate fragments of RNA.

Test polynucleotide molecules that are poorly expressed in particular cells can be enriched using normalization techniques (Bonaldo et al., Genome Res. 6:791-806, 1996).

The test polynucleotides may be detectably labeled at one or more nucleotides. Any method known in the art may be used to detectably label the polynucleotides.

Nucleic acid hybridization and wash conditions are chosen so that the test polynucleotide molecules specifically bind or specifically hybridize to the complementary polynucleotide sequences of the array, preferably to a specific array site, wherein its complementary nucleic acid is located. General parameters for specific (i.e., stringent) hybridization conditions for nucleic acids are described in Sambrook et al., supra, and in Ausubel et al., Current Protocols in Molecular Biology, vol. 2, Current Protocols Publishing, New York, 1994. Typically, stringent conditions for short probes (e.g., 10 to 50 nucleotide bases) will be those in which the salt concentration is at least about 0.01 to 1.0 M at pH 7.0 to 8.3 and the temperature is at least about 30° C. Stringent conditions can also be achieved with the addition of destabilizing agents such as formamide. When fluorescently labeled probes are used, the fluorescence emissions at each site of a microarray can be detected by scanning confocal laser microscopy or other methods (see Shalon et al., Genome Research 6:639-645, 1996; Schena et al., Genome Res. 6:639-645, 1996; and Ferguson et al., Nature Biotech. 14:1681-1684, 1996). Signals are recorded and typically analyzed by computer. Methods for evaluating microarray data and classifying samples are described in U.S. Pat. No. 7,171,311.

Gene expression profiles can also be determined using PCR. PCR is useful to amplify and detect transcripts from a melanoma sample. Various PCR methodologies are useful for gene expression analyses.

Reverse Transcriptase PCR (RT-PCR): RT-PCR is a sensitive quantitative method that can be used to compare mRNA levels in different samples to examine gene expression signatures. To perform RT-PCR, mRNA is isolated from a. mRNA can be extracted, for example, from frozen or archived paraffin-embedded and fixed (e.g. formalin-fixed) tissue samples. Methods for mRNA extraction are known in the art. See, e.g., Ausubel et al., Current Protocols in Molecular Biology, John Wiley and Sons, 1997. Methods for RNA extraction from paraffin embedded tissues are disclosed, for example, in Rupp and Locker, Lab Invest. 56:A67, 1987, and De Andres et al., BioTechniques 18:42044, 1995. Purification kits for RNA isolation from commercial manufacturers, such as Qiagen, can be used. For example, total RNA from a sample can be isolated using Qiagen RNeasy mini-columns. Other commercially available RNA isolation kits include MasterPure™. Complete DNA and RNA Purification Kit (EPICENTRE™, Madison, Wis.), and Paraffin Block RNA Isolation Kit (Ambion, Inc.). Total RNA from tissue samples can be also isolated using RNA Stat-60 (Tel-Test) or by cesium chloride density gradient centrifugation.

Isolated RNA is reverse transcribed into cDNA. The cDNA is amplified in a PCR reaction. Two commonly used reverse transcriptases are avian myeloblastosis virus reverse transcriptase (AMV-RT) and Moloney murine leukemia virus reverse transcriptase (MMLV-RT). The reverse transcription step is typically primed using specific primers, random hexamers, or oligo-dT primers, depending on the conditions and desired readout. For example, extracted RNA can be reverse-transcribed using a GeneAmp RNA PCR kit (Perkin Elmer, Calif., USA), following the manufacturer's instructions. The derived cDNA can then be used as a template in the subsequent PCR reaction. The PCR reaction typically employs the Taq DNA polymerase, which has a 5′-3′ nuclease activity but lacks a 3′-5′ proofreading endonuclease-activity. Two oligonucleotide primers are used to generate an amplicon in the PCR reaction.

Guidelines for PCR primer and probe design are described, e.g., in Dieffenbach et al., “General Concepts for PCR Primer Design” in: PCR Primer, A Laboratory Manual, Cold Spring Harbor Laboratory Press, New York, 133-155, 1995; Innis and Gelfand, “Optimization of PCRs” in: PCR Protocols, A Guide to Methods and Applications, CRC Press, London, 5-11, 1994; and Plasterer, T. N. Primerselect: Primer and probe design. Methods Mol. Biol. 70:520-527, 1997. Factors considered in PCR primer design include primer length, melting temperature (Tₘ), and G/C content, specificity, complementary primer sequences, and 3′-end sequence. PCR primers are generally 17-30 bases in length, and Tₘ's between 50-80° C., e.g. about 50 to 70° C. are typically preferred.

For quantitative PCR, a third oligonucleotide, or probe, is used to detect nucleotide sequence located between the two PCR primers. The probe is non-extendible by Taq DNA polymerase enzyme, and typically is labeled with a reporter fluorescent dye and a quencher fluorescent dye. Any laser-induced emission from the reporter dye is quenched by the quenching dye when the two dyes are located close together as they are on the probe. During the amplification reaction, the Taq DNA polymerase enzyme cleaves the probe in a template-dependent manner. The resultant probe fragments disassociate in solution, and signal from the released reporter dye is free from the quenching effect of the second fluorophore. One molecule of reporter dye is liberated for each new molecule synthesized, and detection of the unquenched reporter dye provides the basis for quantitative analysis.

RT-PCR can be performed using commercially available equipment, such as an ABI PRISM 7700™ Sequence Detection System (Perkin-Elmer-Applied Biosystems, Foster City, Calif., USA), or Lightcycler®. (Roche Molecular Biochemicals, Mannheim, Germany). Samples can be analyzed using a real-time quantitative PCR device such as the ABI PRISM 7700™ Sequence Detection System™.

To minimize errors and the effect of sample-to-sample variation, RT-PCR is usually performed using an internal standard. A suitable internal standard is expressed at a constant level among different tissues, and is unaffected by the experimental variable. RNAs frequently used to normalize patterns of gene expression are mRNAs for the housekeeping genes glyceraldehyde-3-phosphate-dehydrogenase (GAPDH) and β-actin.

A variation of the RT-PCR technique is real time quantitative PCR, which measures PCR product accumulation through a dual-labeled fluorogenic probe (i.e., TaqMan™ probe). Real time PCR is compatible both with quantitative competitive PCR, where internal competitor for each target sequence is used for normalization, and with quantitative comparative PCR using a normalization gene contained within the sample, or a housekeeping gene for RT-PCR. For further details see, e.g. Held et al., Genome Res. 6:986-994, 1996.

Gene expression can be examined using fixed, paraffin-embedded tissues as the RNA source. Briefly, in one exemplary method, sections of paraffin-embedded melanoma tumor tissue samples are cut (^{˜}10 μm thick). RNA is extracted, and protein and DNA are removed. After analysis of the RNA concentration, RNA repair and/or amplification steps may be performed, if necessary, and RNA is reverse transcribed using gene specific promoters followed by RT-PCR. Methods of examining expression in fixed, paraffin-embedded tissues, are described, for example, in Godfrey et al. (J Molec. Diagn. 2: 84-91, 2000) and Specht et. al. (Am. J. Pathol. 158: 419-29, 2001).

Another approach for gene expression analysis employs competitive PCR design and automated, high-throughput matrix-assisted laser desorption ionization time-of-flight (MALDI-TOF) mass spectrometry (MS) detection and quantification of oligonucleotides. This method is described by Ding and Cantor (Proc. Natl. Acad. Sci. U.S.A. 100:3059-3064, 2003). See also the MassARRAY-based gene expression profiling method, developed by Sequenom, Inc. (San Diego, Calif.).

Additional PCR-based techniques for gene expression analysis include, e.g., differential display (Liang and Pardee, Science 257:967-971, 1992); amplified fragment length polymorphism (iAFLP) (Kawamoto et al., Genome Res. 12:1305-1312, 1999); BeadArray™ technology (Illumina, San Diego, Calif.; Oliphant et al., Discovery of Markers for Disease (Supplement to Biotechniques), June 2002; Ferguson et al., Analytical Chemistry 72:5618, 2000); Beads Array for Detection of Gene Expression (BADGE), using the commercially available Luminex100 LabMAP system and multiple color-coded microspheres (Luminex Corp., Austin, Tex.) in a rapid assay for gene expression (Yang et al., Genome Res. 11:1888-1898, 2001); and high coverage expression profiling (HiCEP) analysis (Fukumura et al., Nucl. Acids. Res. 31(16) e94, 2003).

Serial Analysis of Gene Expression (SAGE): Gene expression in melanoma samples can also be determined by serial analysis of gene expression (SAGE), which is a method that allows the simultaneous and quantitative analysis of a large number of gene transcripts, without the need of providing an individual hybridization probe for each transcript (see, e.g. Velculescu et al., Science. 270:484-487, 1995; and Velculescu et al., Cell 88:243-51, 1997). Briefly, a short sequence tag (about 10-14 nucleotides) is generated that contains sufficient information to uniquely identify a transcript, provided that the tag is obtained from a unique position within each transcript. Many transcripts are then linked together to form long serial molecules that can be sequenced, revealing the identity of the multiple tags simultaneously. The expression pattern of a population of transcripts can be quantitatively evaluated by determining the abundance of individual tags, and identifying the gene corresponding to each tag.

Gene expression assays include measures to correct for differences in RNA variability and quality. For example, an assay typically measures and incorporates the expression of certain normalizing genes, such known housekeeping genes, e.g., GAPDH, β-actin, and Cyp1. Alternatively, normalization can be based on the mean or median signal (Ct) of all of the assayed genes or a large subset thereof (global normalization approach). In some embodiments, a normalized test RNA (e.g., from a patient sample) is compared to the amount found in a metastatic melanoma, non-metastatic melanoma, and/or normal skin sample reference set. The level of expression measured in a particular test sample can be determined to fall at some percentile within a range observed in reference sets.

Protein Detection Methodologies: Immunohistochemical methods are also suitable for detecting the expression of melanoma signature genes such as those described herein. Antibodies, most preferably monoclonal antibodies, specific for a gene product are used to detect expression. The antibodies can be detected by direct labeling of the antibodies themselves, for example, with radioactive labels, fluorescent labels, hapten labels such as, biotin, or an enzyme such as horse radish peroxidase or alkaline phosphatase. Alternatively, unlabeled primary antibody is used in conjunction with a labeled secondary antibody, comprising antisera, polyclonal antisera or a monoclonal antibody specific for the primary antibody. Immunohistochemistry protocols and kits are well known in the art and are commercially available.

Proteomic methods can allow examination of global changes in protein expression in a sample. Proteomic analysis typically involves separation of individual proteins in a sample by 2-D gel electrophoresis (2-D PAGE), and identification of individual proteins recovered from the gel, e.g. my mass spectrometry or N-terminal sequencing, and analysis of the data using bioinformatics. Proteomics methods can be used alone or in combination with other methods for evaluating gene expression.

In various aspects, the expression of certain genes in a cutaneous sample is detected to provide clinical information (e.g., prognostic information, classification of the melanoma tumor from which the sample is derived, as a melanoma associated with prolonged or truncated longevity).

The following Tables provide representative subsets of the genes listed in Table 5, which subsets optionally may be used in gene expression profiles as described herein. The direction of expression change (expression delta, compared to expression in a reference group of melanomas) that indicates high (or relatively higher) risk of melanoma metastasis is indicated. Other subsets are also contemplated, including any five, any eight, any 10, any 15, any 20, any 25, any 30, or more of the genes listed in Table 5, or in any of Tables 2-4 or combinations thereof.

TABLE 1

| | | Direction of |
|---|---|---|
| Gene | | Expression in High |
| Symbol | Gene Title | risk Melanoma |
| BAP1 | BRCA1-associated protein-1 | Decreased |
| MGP | Matrix Gla protein | Decreased |
| SPP1 | Secreted phosphoprotein 1 | Increased |
| CXCL14 | Chemokine (C-X-C motif) ligand 14 | Decreased |
| CLCA2 | Chloride channel accessory 2 | Decreased |
| S100A8 | S100 calcium-binding protein A8 | Decreased |
| BTG1 | B-cell translocation gene 1, | Decreased |
| | antiproliferative | |
| SAP130 | Sin3A-associated protein, 130 kDa | Decreased |
| ARG1 | Arginase 1 | Decreased |
| KRT6B | Keratin 6B | Increased |
| GJA1 | Gap junction protein, alpha 1, 43 kDa | Decreased |
| ID2 | Inhibitor of DNA binding 2, dominant | Decreased |
| | negative helix-loop-helix protein | |
| EIF1B | Eukaryotic translation initiation factor | Increased |
| | 1B | |
| S100A9 | S100 calcium-binding protein A9 | Decreased |
| CRABP2 | Cellular retinoic acid binding protein 2 | Decreased |
| KRT14 | Keratin 14 | Decreased |
| ROBO1 | Roundabout, axon guidance receptor, | Decreased |
| | homolog 1 | |
| RBM23 | RNA-binding motif protein 23 | Decreased |
| TACSTD2 | Tumor-associated calcium signal | Decreased |
| | transducer 2 | |
| DSC1 | Desmocollin 1 | Decreased |
| SPRR1B | Small proline-rich protein 1B | Decreased |
| TRIM29 | Tripartite motif containing 29 | Decreased |
| AQP3 | Aquaporin 3 (Gill blood group) | Decreased |
| TYRP1 | Tyrosinase-related protein 1 | Decreased |
| PPL | Periplakin | Decreased |
| LTA4H | Leukotriene A4 hydrolase | Decreased |
| CST6 | Cystatin E/M | Decreased |

TABLE 2

| | | Direction of |
|---|---|---|
| Gene | | Expression in High |
| Symbol | Gene Title | risk Melanoma |
| CXCL8 | Interleukin 8 | Increased |
| ITGB3 | Integrin beta 3 | Increased |
| LAMB1 | laminin B1 chain | Increased |
| PLAT | tissue-type plasminogen activator | Increased |
| TP53 | Cellular tumor antigen p53 | Decreased |

TABLE 3

| | | Direction of |
|---|---|---|
| Gene | | Expression in High |
| Symbol | Gene Title | risk Melanoma |
| ANGPT1 | Angiopoietin 1 | Increased |
| ANGPT2 | Angiopoietin 2 | Increased |
| BMP2 | Bone morphogenic protein 2 | Decreased |
| FGF2 | Fibroblast Growth Factor 2 | Increased |
| S1PR1 | Sphingosine 1-phosphate | Increased |
| TGFB1 | Transforming growth factor beta | Decreased |
| VEGFA | Vascular Endothelial Growth Factor A | Increased |
| VEGFC | Vascular Endothelial Growth Factor C | Increased |
| VEGFD | Vascular Endothelial Growth Factor D | Increased |
| IFNG | Interferon-gamma | Decreased |

TABLE 4

| | | Direction of |
|---|---|---|
| Gene | | Expression in High |
| Symbol | Gene Title | risk Melanoma |
| GDF15 | Growth Differentiation Factor 15 | Increased |
| MLANA | Melan-A | Increased |
| PLAT | tissue-type plasminogen activator | Increased |
| CXCL8/IL8 | Interleukin 8 | Increased |
| ITGB3 | Integrin beta 3 | Increased |
| LOXL4 | Lysyl oxidase like 4 | Increased |
| PRKCB | protein kinase C-β | Increased |
| SERPINE2 | glia-derived nexin | Increased |
| ADAM12 | ADAM metallopeptidase domain 12 | Increased |
| LGALSI | galectin 1 | Increased |
| TGFBRI | transforming growth factor-β | Increased |
| | (TGF-β) receptor 1 | |

TABLE 5
Gene List including Direction of Expression in High-risk Melanoma

| Gene | Entrez | | |
|---|---|---|---|
| Symbol | Gene ID | Gene Title/Name | Direction |
| ABCC12 | 94160 | Multidrug resistance-associated protein 9 | Decreased |
| ACOT1 | 641371 | Acyl-coenzyme A thioesterase 1 | Decreased |
| ADAM12 | 8038 | ADAM metallopeptidase domain 12 | Increased |
| AGBL4 | 84871 | Cytosolic carboxypeptidase 6 | Increased |
| AGXT | 189 | Alanine-glyoxylate aminotransferase | Decreased |
| AIM2 | 9447 | Interferon-inducible protein AIM2 | Decreased |
| ALK | 238 | ALK tyrosine kinase receptor | Decreased |
| ANGPT1 | 284 | Angiopoietin 1 | Increased |
| ANGPT2 | 285 | Angiopoietin 2 | Increased |
| ANGPTL7 | 10218 | Angiopoietin-related protein 7 | Increased |
| ANK1 | 286 | Ankyrin-1 | Decreased |
| AQP3 | 360 | Aquaporin 3 (Gill blood group) | Decreased |
| ARG1 | 383 | Arginase 1 | Decreased |
| ARRDC1 | 92714 | Arrestin domain-containing protein 1 | Decreased |
| ART1 | 417 | GPI-linked NAD(P)(+)-arginine ADP- | Decreased |
| | | ribosyltransferase 1 | |
| BAP1 | 8314 | BRCA1-associated protein-1 | Decreased |
| BIN1 | 274 | Myc box-dependent-interacting protein 1 | Decreased |
| BMP2 | 650 | Bone morphogenic protein 2 | Decreased |
| BMX | 660 | Cytoplasmic tyrosine-protein kinase BMX | Decreased |
| BTG1 | 694 | B-cell translocation gene 1, antiproliferative | Decreased |
| C8G | 733 | Complement component C8 gamma chain | Decreased |
| CACNG4 | 27092 | Voltage-dependent calcium channel γ-4 subunit | Increased |
| CAMK2B | 816 | Calcium/calmodulin-dependent protein kinase type II | Decreased |
| | | subunit beta | |
| CASQ1 | 844 | Calsequestrin-1 | Decreased |
| CCR3 | 1232 | C-C chemokine receptor type 3 | Decreased |
| CCR5 | 1234 | C-C chemokine receptor type 5 | Decreased |
| CDC5L | 988 | Cell division cycle 5-like protein | Increased |
| CENPQ | 55166 | Centromere protein Q | Increased |
| CETP | 1071 | Cholesteryl ester transfer protein | Decreased |
| CLCA2 | 9635 | Chloride channel accessory 2 | Decreased |
| CLIC5 | 53405 | Chloride intracellular channel protein 5 | Decreased |
| COL24A1 | 255631 | Collagen alpha-1(XXIV) chain | Increased |
| CPN2 | 1370 | Carboxypeptidase N subunit 2 | Increased |
| CRABP2 | 1382 | Cellular retinoic acid binding protein 2 | Decreased |
| CST6 | 1474 | Cystantin E/M | Decreased |
| CTAGE1 | 64693 | cTAGE family member 2 | Decreased |
| CTCFL | 140690 | Transcriptional repressor CTCFL | Increased |
| CUL7 | 9820 | Cullin-7 | Increased |
| CXCL14 | 9547 | Chemokine (C-X-C motif) ligand 14 | Decreased |
| CXCL8/IL8 | 3576 | Interleukin 8 | Increased |
| DIO3OS | 64150 | DIO3 opposite strand upstream RNA | Decreased |
| | | [Source: HGNC Symbol; Acc: HGNC: 20348] | |
| DMAP1 | 55929 | DNA methyltransferase 1-associated protein 1 | Increased |
| DOCK3 | 1795 | Dedicator of cytokinesis protein 3 | Decreased |
| DPEP3 | 64180 | Dipeptidase 3 | Increased |
| DSC1 | 1823 | Desmocollin 1 | Decreased |
| DYSF | 8291 | Dysferlin | Decreased |
| EEF1A2 | 1917 | Elongation factor 1-alpha 2 | Decreased |
| EHBP1L1 | 254102 | EH domain-binding protein 1-like protein 1 | Decreased |
| EIF1B | 10289 | Eukaryotic translation initiation factor 1B | Increased |
| ERGIC2 | 51290 | Endoplasmic reticulum-Golgi intermediate | Increased |
| | | compartment protein 2 | |
| F7 | 2155 | Coagulation factor VII | Decreased |
| FASLG | 356 | Tumor necrosis factor ligand superfamily member 6 | Decreased |
| FGF2 | 2247 | Fibroblast Growth Factor 2 | Increased |
| FLOT1 | 10211 | Flotillin-1 | Increased |
| FLVCR2 | 55640 | Feline leukemia virus subgroup C receptor-related | Decreased |
| | | protein 2 | |
| FNBP1L | 54874 | Formin-binding protein 1-like | Increased |
| FREM2 | 341640 | FRAS1-related extracellular matrix protein 2 | Decreased |
| GABBR2 | 9568 | Gamma-aminobutyric acid type B receptor subunit 2 | Decreased |
| GBP5 | 115362 | Guanylate-binding protein 5 | Decreased |
| GCH1 | 2643 | GTP cyclohydrolase 1 | Decreased |
| GDF15 | 9518 | Growth Differentiation Factor 15 | Increased |
| GFRA1 | 2674 | GDNF family receptor alpha-1 | Decreased |
| GJA1 | 2697 | Gap junction protein, alpha 1, 43 kDa | Decreased |
| GLDC | 2731 | Glycine dehydrogenase (decarboxylating), | Decreased |
| | | mitochondrial | |
| GPR39 | 2863 | G-protein coupled receptor 39 | Decreased |
| GPR63 | 81491 | Probable G-protein coupled receptor 63 | Decreased |
| GRAMD1B | 57476 | GRAM domain containing 1B | Decreased |
| HEPACAM | 220296 | Hepatocyte cell adhesion molecule | Decreased |
| HHATL | 57467 | Protein-cysteine N-palmitoyltransferase HHAT-like | Increased |
| | | protein | |
| HMGCR | 3156 | 3-hydroxy-3-methylglutaryl-CoA reductase | Increased |
| HNF4A | 3172 | Hepatocyte nuclear factor 4-alpha | Increased |
| HTR3B | 9177 | 5-hydroxytryptamine receptor 3B | Decreased |
| ID2 | 3398 | Inhibitor of DNA binding 2, dominant negative helix- | Decreased |
| | | loop-helix protein | |
| IFNG | 3458 | Interferon-gamma | Decreased |
| ITGB3 | 3690 | Integrin beta 3 | Increased |
| JARID2 | 3720 | Protein Jumonji | Increased |
| JPH3 | 57338 | Junctophilin-3 | Decreased |
| KCNT1 | 57582 | Potassium channel subfamily T member 1 | Decreased |
| KIF19 | 124602 | Kinesin-like protein KIF19 | Decreased |
| KLC4 | 89953 | Kinesin light chain 4 | Increased |
| KLHDC8A | 55220 | Kelch domain containing 8A | Decreased |
| KNDC1 | 85442 | Protein very KIND | Decreased |
| KRT14 | 3861 | Keratin 14 | Decreased |
| KRT6B | 3854 | Keratin 6B | Increased |
| KRTAP19-6 | 337973 | Keratin-associated protein 19-6 | Increased |
| KSR2 | 283455 | Kinase suppressor of Ras 2 | Decreased |
| KYNU | 8942 | Kynureninase | Decreased |
| LAG3 | 3902 | Lymphocyte activation gene 3 protein | Decreased |
| LAMB1 | 3912 | laminin B1 chain | Increased |
| LGALSI | 3956 | galectin 1 | Increased |
| LGI4 | 163175 | Leucine rich repeat LGI family member 4 | Increased |
| LRRC31 | 79782 | Leucine-rich repeat-containing protein 31 | Decreased |
| LTA4H | 4048 | Leukotriene A4 hydrolase | Decreased |
| LOXL4 | 84171 | Lysyl oxidase like 4 | Increased |
| MCF2 | 4168 | Proto-oncogene DBL | Decreased |
| ME3 | 10873 | NADP-dependent malic enzyme, mitochondrial | Decreased |
| MFSD6L | 162387 | Major facilitator superfamily domain containing 6 like | Decreased |
| MGP | 4256 | Matrix Gla protein | Decreased |
| MIR222 | 407007 | microRNA 222 | Decreased |
| | | [Source: HGNC Symbol; Acc: HGNC: 31602] | |
| MLANA | 2315 | Melan-A | Increased |
| MLF1 | 4291 | Myeloid leukemia factor 1 | Increased |
| MTUS2 | 23281 | Microtubule-associated tumor suppressor candidate 2 | Decreased |
| NFYA | 4800 | Nuclear transcription factor Y subunit alpha | Increased |
| NPPC | 4880 | C-type natriuretic peptide | Decreased |
| NTRK1 | 4914 | High affinity nerve growth factor receptor | Decreased |
| NXPH1 | 30010 | Neurexophilin-1 | Increased |
| OCIAD2 | 132299 | OCIA domain containing 2 | Decreased |
| OR52K1 | 390036 | Olfactory receptor 52K1 | Decreased |
| OR5AK2 | 390181 | Olfactory receptor 5AK2 | Decreased |
| P2RY14 | 9934 | P2Y purinoceptor 14 | Decreased |
| PARP11 | 57097 | Poly [ADP-ribose] polymerase 11 | Decreased |
| PHEX | 5251 | Phosphate-regulating neutral endopeptidase | Decreased |
| PIK3R6 | 146850 | Phosphoinositide 3-kinase regulatory subunit 6 | Decreased |
| PLA2G2D | 26279 | Group IID secretory phospholipase A2 | Decreased |
| PLAT | 5327 | tissue-type plasminogen activator | Increased |
| PLCZ1 | 89869 | 1-phosphatidylinositol 4,5-bisphosphate | Decreased |
| | | phosphodiesterase zeta-1 | |
| PPL | 5493 | Periplakin | Decreased |
| PRF1 | 5551 | Perforin-1 | Decreased |
| PRKCB | 5579 | protein kinase C-β | Increased |
| PROCA1 | 147011 | Protein interacting with cyclin A1 | Decreased |
| PTPRG | 5793 | Receptor-type tyrosine-protein phosphatase γ | Increased |
| RAB15 | 376267 | Ras-related protein Rab-15 | Decreased |
| RBBP4 | 5928 | Histone-binding protein RBBP4 | Increased |
| RBM23 | 55147 | RNA-binding motif protein 23 | Decreased |
| RGS7 | 6000 | Regulator of G-protein signaling 7 | Decreased |
| RNF213 | 57674 | E3 ubiquitin-protein ligase RNF213 | Decreased |
| ROBO1 | 6091 | Roundabout, axon guidance receptor, homolog 1 | Decreased |
| RP1 | 6101 | Oxygen-regulated protein 1 | Increased |
| RPL7L1 | 285855 | Ribosomal protein L7 like 1 | Increased |
| S100A8 | 6279 | S100 calcium-binding protein A8 | Decreased |
| S100A9 | 6280 | S100 calcium-binding protein A9 | Decreased |
| S1PR1 | 1901 | Sphingosine 1-phosphate | Increased |
| SAP130 | 79595 | Sin3A-associated protein, 130 kDa | Decreased |
| SERPINE2 | 5270 | glia-derived nexin | Increased |
| SFMBT2 | 57713 | Scm-like with four MBT domains protein 2 | Decreased |
| SIGLEC12 | 89858 | Sialic acid-binding Ig-like lectin 12 | Decreased |
| SLC17A3 | 10786 | Sodium-dependent phosphate transport protein 4 | Increased |
| SLC29A1 | 2030 | Equilibrative nucleoside transporter 1 | Increased |
| SLCO5A1 | 81796 | Solute carrier organic anion transporter family | Decreased |
| | | member 5A1 | |
| SLIT1 | 6585 | Slit homolog 1 protein | Decreased |
| SOCS1 | 8651 | Suppressor of cytokine signaling 1 | Decreased |
| SOX4 | 6659 | Transcription factor SOX-4 | Increased |
| SPAG6 | 9576 | Sperm-associated antigen 6 | Decreased |
| SPP1 | 6696 | Secreted phosphoprotein 1 | Increased |
| SPRR1B | 6699 | Small proline-rich protein 1B | Decreased |
| STMN1 | 3925 | Stathmin | Increased |
| TACSTD2 | 4070 | Tumor-associated calcium signal transducer 2 | Decreased |
| TAS2R60 | 338398 | Taste receptor type 2 member 60 | Decreased |
| TDRD12 | 91646 | Putative ATP-dependent RNA helicase TDRD12 | Increased |
| TFF2 | 7032 | Trefoil factor 2 | Decreased |
| TGFB1 | 7040 | Transforming growth factor beta | Decreased |
| TGFBRI | 7046 | transforming growth factor-β (TGF-β) receptor 1 | Increased |
| TLR4 | 7099 | Toll-like receptor 4 | Decreased |
| TMCC3 | 57458 | Transmembrane and coiled-coil domain containing | Decreased |
| TNFRSF10A | 8797 | Tumor necrosis factor receptor superfamily member | Decreased |
| | | 10A | |
| TP53 | 7157 | Cellular tumor antigen p53 | Decreased |
| TRIM22 | 10346 | E3 ubiquitin-protein ligase TRIM22 | Decreased |
| TRIM29 | 23650 | Tripartite motif containing 29 | Decreased |
| TRNAU1AP | 54952 | tRNA selenocysteine 1-associated protein 1 | Increased |
| TTN | 7273 | Titin | Decreased |
| TUBB | 203068 | Tubulin beta chain | Increased |
| TYRP1 | 7306 | Tyrosinase-related protein 1 | Decreased |
| UTS2 | 10911 | Urotensin 2 | Decreased |
| VAMP5 | 10791 | Vesicle-associated membrane protein 5 | Decreased |
| VEGFA | 7422 | Vascular Endothelial Growth Factor A | Increased |
| VEGFC | 7424 | Vascular Endothelial Growth Factor C | Increased |
| VEGFD | 2277 | Vascular Endothelial Growth Factor D | Increased |
| WDR49 | 151790 | WD repeat-containing protein 49 | Decreased |
| WIF1 | 11197 | Wnt inhibitory factor 1 | Decreased |
| ZIM2 | 23619 | Zinc finger imprinted 2 | Increased |
| ZNF560 | 147741 | Zinc finger protein 560 | Increased |
| ZNF697 | 90874 | Zinc finger protein 697 | Increased |

### VI. Melanoma Treatment Including Combination Therapies

Excisional biopsies may remove the tumor, but further surgery is often necessary to reduce the risk of recurrence. Complete surgical excision with adequate surgical margins and assessment for the presence of detectable metastatic disease along with short- and long-term follow-up is standard. Often this is done by a wide local excision (WLE) with 1 to 2 cm margins. Melanoma-in situ and lentigo malignas are treated with narrower surgical margins, usually 0.2 to 0.5 cm. Many surgeons consider 0.5 cm the standard of care for standard excision of melanoma-in-situ, but 0.2 cm margin might be acceptable for margin controlled surgery (Mohs surgery, or the double-bladed technique with margin control). The wide excision aims to reduce the rate of tumor recurrence at the site of the original lesion. This is a common pattern of treatment failure in melanoma. Considerable research has aimed to elucidate appropriate margins for excision with a general trend toward less aggressive treatment during the last decades.

Mohs surgery has been reported with cure rate as low as 77% and as high as 98% for melanoma-in-situ. CCPDMA and the “double scalpel” peripheral margin controlled surgery is equivalent to Mohs surgery in effectiveness on this “intra-epithelial” type of melanoma.

Melanomas that spread usually do so to the lymph nodes in the area of the tumor before spreading elsewhere. Attempts to improve survival by removing lymph nodes surgically (lymphadenectomy) were associated with many complications, but no overall survival benefit. Recently, the technique of sentinel lymph node biopsy has been developed to reduce the complications of lymph node surgery while allowing assessment of the involvement of nodes with tumor.

Sentinel lymph node biopsy is often performed, especially for T1b/T2+ tumors, mucosal tumors, ocular melanoma and tumors of the limbs. A process called lymphoscintigraphy is performed in which a radioactive tracer is injected at the tumor site to localize the sentinel node(s). Further precision is provided using a blue tracer dye, and surgery is performed to biopsy the node(s). Routine hematoxylin and eosin (H&E) and immunoperoxidase staining will be adequate to rule out node involvement. Polymerase chain reaction (PCR) tests on nodes, usually performed to test for entry into clinical trials, now demonstrate that many patients with a negative sentinel lymph node actually had a small number of positive cells in their nodes. Alternatively, a fine-needle aspiration biopsy may be performed and is often used to test masses.

If a lymph node is positive, depending on the extent of lymph node spread, a radical lymph node dissection will often be performed. If the disease is completely resected, the patient will be considered for adjuvant therapy. Excisional skin biopsy is the management of choice. The suspect lesion is totally removed with an adequate (but minimal, usually 1 or 2 mm) ellipse of surrounding skin and tissue. To avoid disruption of the local lymphatic drainage, the preferred surgical margin for the initial biopsy should be narrow (1 mm). The biopsy should include the epidermal, dermal, and subcutaneous layers of the skin. This enables the histopathologist to determine the thickness of the melanoma by microscopic examination. This is described by Breslow's thickness (measured in millimeters). However, for large lesions, such as suspected lentigo maligna, or for lesions in surgically difficult areas (face, toes, fingers, eyelids), a small punch biopsy in representative areas will give adequate information and will not disrupt the final staging or depth determination. In no circumstances should the initial biopsy include the final surgical margin (0.5 cm, 1.0 cm, or 2 cm), as a misdiagnosis can result in excessive scarring and morbidity from the procedure. A large initial excision will disrupt the local lymphatic drainage and can affect further lymphangiogram-directed lymph node dissection. A small punch biopsy can be used at any time where for logistical and personal reasons a patient refuses more invasive excisional biopsy. Small punch biopsies are minimally invasive and heal quickly, usually without noticeable scarring.

High-risk melanomas may require adjuvant treatment. Patients in otherwise good health may begin up to a year of high-dose interferon treatment, which may improve the patient's prognosis slightly. A 2011 meta-analysis showed that interferon could lengthen the time before a melanoma comes back but increased survival by only 3% at 5 years. Unpleasant side effects may decrease quality of life.

Various chemotherapy agents also are used, including dacarbazine (also termed DTIC), immunotherapy (with interleukin-2 (IL-2) or interferon (IFN)), as well as local perfusion. The overall success in metastatic melanoma is quite limited. IL-2 (PROLEUKIN@) is the first new therapy approved for the treatment of metastatic melanoma in 20 years. Studies have demonstrated that IL-2 offers the possibility of a complete and long-lasting remission in this disease, although only in a small percentage of patients.

For lentigo maligna treatment, standard excision is still being performed by most surgeons. Unfortunately, the recurrence rate is exceedingly high (up to 50%). This is due to the ill-defined visible surgical margin, and the facial location of the lesions (often forcing the surgeon to use a narrow surgical margin). The narrow surgical margin used, combined with the limitation of the standard “bread-loafing” technique of fixed tissue histology, result in a high “false negative” error rate, and frequent recurrences. Margin control (peripheral margins) is necessary to eliminate the false negative errors. If bread loafing is used, distances from sections should approach 0.1 mm to assure that the method approaches complete margin control.

Some melanocytic nevi, and melanoma-in-situ (lentigo maligna), have resolved with an experimental treatment: imiquimod (ALDARA@) topical cream, an immune enhancing agent. The two methods, surgically excising the cancer and then treating the area with ALDARA@cream postoperatively for three months, are sometimes combined.

Radiation therapy is often used after surgical resection for patients with locally or regionally advanced melanoma, or for patients with unresectable distant metastases. It may reduce the rate of local recurrence but does not prolong survival. Radioimmunotherapy of metastatic melanoma is currently under investigation. Radiotherapy has a role in the palliation of metastatic melanoma.

The results described herein demonstrate that statins can beneficially be used in conjunction with other cancer treatments, for instance as a combination therapy (though the compounds need not be co-administered). For example, the statins can be administered in combination with other active ingredients, for example, an AR antagonist (e.g., Enz, darolutamide, proxalutamide, apalutamide, biulatamide) a gonadotropin-releasing hormone agonist or antagonist (e.g., Lupron, ZOLADEX@ (Goserelin), Degarelix, Ozarelix, ABT-620 (Elagolix), TAK-385 (Relugolix), EP-100 or KLH-2109); a phosphoinositide 3-kinase (P13K) inhibitor, a TORC inhibitor, or a dual PI3K/TORC inhibitor (e.g., BEZ-235, BKM120, BGT226, BYL-719, GD00068, GDC-0980, GDC0941, GD00032, MK-2206, OSI-027, CC-223, AZD8055, SAR245408, SAR245409, PF04691502, WYE125132, GSK2126458, GSK-2636771, BAY806946, PF-05212384, SF1126, PX866, AMG319, ZSTK474, Cal101, PWT33597, LY-317615 (enzastaurin hydrochloride), CU-906, or CUDC-907); a CYP17 inhibitor in addition to Galeterone (e.g., abiraterone acetate (Zytiga), TAK-700 (orteronel), or VT-464); prednisone; an osteoprotective agent; a radiation therapy; a kinase inhibitor (e.g. MET, VEGFR, EGFR, MEK, SRC, AKT, RAF, FGFR, CDK4/6); PROVENGE@, Prostvac, Ipilimumab, a PD-1 inhibitor; a taxane or tubulin inhibitor; an anti-STEAP-1 antibody; a heat shock protein 90 (HSP90) or heat shock protein 27 (HSP27) pathway modulator; and/or immunotherapy.

Particularly contemplated are combination therapy that includes treatment with a statin and a BRAF inhibitor, or a statin and an immunotherapeutic agent, or a stating and any therapeutic agent known or discovered to be useful in the treatment of melanoma.

BRAF (also referred to as proto-oncogene B-Raf and v-Raf murine sarcoma viral oncogene homolog B1) is a human gene that makes the protein B-Raf (serine/threonine-protein kinase B-Raf). The B-Raf protein is involved in sending signals inside cells, which are involved in directing cell growth. It has been shown to be faulty (mutated) in human cancers. Drugs that treat cancers driven by BRAF have been developed. Vemurafenib was approved by FDA in 2011 for treatment of late-stage melanoma as the first drug to come out of fragment-based drug discovery. Pharmaceutical companies are developing other inhibitors of mutated B-raf protein for anticancer use, including dabrafenib. More general B-raf inhibitors include GDC-0879, PLX 4032, PLX-4720, PLX 4734 and Sorafenib Tosylate. See also U.S. Pat. No. 9,561,245. The combination of a MEK inhibitor (trametinib) with a RAF inhibitor (dabrafenib) is now an approved therapy for BRAF mutation-positive metastatic melanoma (reviewed in Spain et al, Expert Opin Pharmacother 17, 1031-1038, 2016).

The dosage of the individual BRAF inhibitors used in the pharmaceutical combination may be equal to or lower than the dose of an individual therapeutic agent when given independently to treat, manage, or ameliorate a disease or disorder, or one or more symptoms thereof. In an embodiment of the invention, the disease or disorder being treated with a combination therapy is a proliferative disorder, such as melanoma. In an embodiment, the proliferative disorder is cancer. In an embodiment, the BRAF inhibitor PLX-4032 is administered at a dose of between about 200 mg to about 2000 mg. In an embodiment, PLX-4032 is administered at a dose from about 480 mg to about 960 mg. In an embodiment, PLX-4032 is administered orally at a dose from about 480 mg to about 960 mg. In an embodiment, PLX-4032 is administered orally at a dose from about 480 mg to about 960 mg twice daily. In an embodiment, PLX-4032 is administered at a dose of about 480 mg twice daily. In an embodiment, PLX-4032 is administered at about 720 mg twice daily. In an embodiment, PLX-4032 is administered at about 960 mg twice daily. The recommended dosages of therapeutic agents currently used for the treatment, management, or amelioration of a disease or disorder, or one or more symptoms thereof, can obtained from any reference in the art. For a more in depth review of dosage and treatment schedules for various disorders, see, e.g., Goodman & Gilman's The Pharmacological Basis of Therapeutics 9^{th} Ed. (Hardman et al., Eds., NY: Mc-Graw-Hill (1996)); Physician's Desk Reference 57^{th} ED. (Medical Economics Co., Inc., Montvale, N.J. (2003)). See also U.S. Pat. No. 9,402,831.

Representative immunotherapeutic agents including, but not limited to, immunostimulants (e.g., Bacille Calmette-Guerin (BCG), levamisole, interleukin-2, alpha-interferon, etc.), therapeutic monoclonal antibodies (e.g., anti-CD20, anti-HER2, anti-CD52, anti-HLA-DR, and anti-VEGF monoclonal antibodies), immunotoxins (e.g., anti-CD33 monoclonal antibody-calicheamicin conjugate, anti-CD22 monoclonal antibody-pseudomonas exotoxin conjugate, etc.), immune-checkpoint inhibitors (e.g., anti-CTLA4, anti-PD1, antiPD-L1 antibodies), and radioimmunotherapy (e.g., anti-CD20 monoclonal antibody conjugated to ¹¹¹In, ⁹⁰Y, or ¹³¹I, etc.). These immunotherapeutic agents can also be loaded directly onto the immunotherapeutic constructs to enhance their therapeutic effect, reduce toxicity, and reduce administration time.

Checkpoint inhibitor therapy is a recently developed and still developing form of cancer immunotherapy currently under research. The therapy targets immune checkpoints, key regulators of the immune system that stimulate or inhibit its actions, which tumors can use to protect themselves from attacks by the immune system. Checkpoint therapy can block inhibitory checkpoints, restoring immune system function (Pardoll, Nature Revs. Cancer 12(4):252-264, 2012). The first anti-cancer drug targeting an immune checkpoint was ipilimumab, a CTLA4 blocker approved in the United States in 2011 (Cameron et al., Drugs 71(8):1093-1104, 2011). See also Wieder et al., J Allergy Clin Immunol. 142(5): 1403-1414, 2018.

Currently approved checkpoint inhibitors target the molecules CTLA4, PD-1, and PD-L1. PD-1 is the transmembrane programmed cell death 1 protein (also called PDCD1 and CD279), which interacts with PD-L1 (PD-1 ligand 1, or CD274). PD-L1 on the cell surface binds to PD1 on an immune cell surface, which inhibits immune cell activity. Among PD-L1 functions is a key regulatory role on T cell activities (Butte et al., Immunity 27(11); 111-122, 2007; Karwacz et al., EMBO Mol. Med. 3(10:581-592, 2011). It appears that cancer-mediated upregulation of PD-L1 on the cell surface may inhibit T cells that might otherwise attack cancer cells. Antibodies that bind to either PD-1 or PD-L1 and therefore block the interaction may allow the T-cells to attack the tumor (Syn et al., The Lancet Oncology 18(12): e731-e741, 2017).

In the immune system, the critical balance between rejection and self-tolerance is maintained by a finely tuned series of co-regulatory receptor-ligand interactions. Recent attention has focused on the programmed death (PD)-1/PD-1 ligand (PD-L1, B7-H1) pathway as a key mediator of tumor immune tolerance. Under physiologic conditions, the inhibitory PD-1 receptor is expressed on activated immune effector cells, including T, B and NK cells. Through interactions with its ligands PD-L1 and PD-L2, normally expressed on antigen presenting cells (APCs), immune effector activity in peripheral tissues during inflammatory processes is self-limited. This inhibitory system is fundamental to protecting healthy tissues and non-infected cells during clearance of viral and bacterial intracellular infections. However, many human cancers have been shown to express PD-1 ligands, thus inducing immune tolerance locally in the tumor microenvironment (TME) and facilitating tumor cell escape from immune attack. Two general mechanisms promoting expression of PD-L1 on tumor cells have been postulated. In some tumors, aberrant signaling pathways can constitutively up-regulate PD-L1 expression, a phenomenon termed “innate immune resistance”; in others, the expression of PD-L1 is an adaptive mechanism that occurs in response to inflammatory cytokines produced in the TME during an antitumor immune response (“adaptive immune resistance”). These mechanisms of PD-L1 expression are not mutually exclusive, i.e., constitutive PD-L1 expression on tumor cells may be further up-regulated by cytokines such as interferon-gamma (IFN-g).

PD-L1 expression by tumor cells prior to treatment correlates highly with response to anti-PD-1 monotherapy (for example, nivolumab (Bristol-Myers Squibb; OPDIVO™) pembrolizumab (Merck; KEYTRUDA®)) and anti-PD-L1 therapy (for example, MPDL3280A (Genentech/Roche)). Additional checkpoint inhibitors include: ipilimumab and tremelimumab (which target CTLA-4); atezolizumab (Genentech/Roche; Tecentriq), avelumab (Merck; Bavencio), and durvalumab (Medimmune/Strazeneca; Imfinzi) (which target PD-L1); and cemiplimab (REGN-2810), nivolumab, pembrolizumab, and pidilizumab (which target PD-1). Spartalizumab (PDR001; Novartis) is also under development as a PD-1 inhibitor.

Methods of PD-1 blockade treatment, including treatment of cancers, are well known in the art. See, for instance, WO 2016/201425, US 2019/0275705, Kvistborg et al. (Science Trans/Med. 6(254):254ra128, 2014), Zou et al. (Science Trans/Med. 8(328):328rv4, 2016), and Sakuishi et al. J Exp Med. 207(10):2187-2194, 2010).

PD-1 blocking agents include those used to treat cancer (i.e., to inhibit the growth or survival of tumor cells). Cancers whose growth may be inhibited using antibodies or anti-PD-1 agents or other check point inhibitors include cancers typically responsive to immunotherapy, but also cancers that have not hitherto been associated with immunotherapy. Examples of cancers for treatment include melanoma (e.g., metastatic malignant melanoma), renal cancer (e.g., clear cell carcinoma), prostate cancer (e.g., hormone refractory prostate adenocarcinoma), pancreatic adenocarcinoma, breast cancer, colon cancer, lung cancer (e.g., non-small cell lung cancer), esophageal cancer, squamous cell carcinoma of the head and neck, liver cancer, ovarian cancer, cervical cancer, thyroid cancer, glioblastoma, glioma, leukemia, lymphoma, and other neoplastic malignancies. The herein described treatments are applicable to malignancies that demonstrate improved disease-free and overall survival in relation to the presence of tumor-infiltrating lymphocytes in biopsy or surgical material, e.g., melanoma, colorectal, liver, kidney, stomach/esophageal, breast, pancreas, and ovarian cancer. Such cancer subtypes are known to be susceptible to immune control by T lymphocytes. Additionally, the provided technology is useful for treating refractory or recurrent malignancies whose growth may be inhibited using the PD-1 or other check point blockade treatments. Particularly cancers include those characterized by elevated expression of PD-1 and/or its ligands PD-L1 and/or PD-L2 in tested tissue samples, including: ovarian, renal, colorectal, pancreatic, breast, liver, glioblastoma, non-small cell lung cancer, gastric, esophageal cancers and melanoma.

The PD-1/PD-L1 pathway is a well-validated target for the development of antibody therapeutics for cancer treatment. Anti-PD-1 antibodies may also be useful in chronic viral infection. Memory CD8+ T cells generated after an acute viral infection are highly functional and constitute an important component of protective immunity. In contrast, chronic infections are often characterized by varying degrees of functional impairment (exhaustion) of virus-specific T-cell responses, and this defect is a principal reason for the inability of the host to eliminate the persisting pathogen. Although functional effector T cells are initially generated during the early stages of infection, they gradually lose function during the course of a chronic infection. Barber et al. (Nature 439: 682-687, 2006) showed that mice infected with a laboratory strain of LCMV developed chronic infection resulting in high levels of virus in the blood and other tissues. These mice initially developed a robust T cell response, but eventually succumbed to the infection upon T cell exhaustion. The authors found that the decline in number and function of the effector T cells in chronically infected mice could be reversed by injecting an antibody that blocked the interaction between PD-I and PD-L1.

There are expressly provided embodiments in which a melanoma patient is treated with a statin, but is not treated with a ROR-gamma (RORγ) inhibitor. This is in contrast with the teachings of WO 2020/047487 (“Methods of Treating Cancer with RORgamma Inhibitors and Statins”). Thus, excluded from the subject matter of this disclosure are combination treatments involving a statin and a RORγ inhibitor.

The Exemplary Embodiments and Experimental Examples below are included to demonstrate particular embodiments of the disclosure. Those of ordinary skill in the art will recognize in light of the present disclosure that many changes can be made to the specific embodiments disclosed herein and still obtain a like or similar result without departing from the spirit and scope of the disclosure.

### VII. Exemplary Embodiments

Set 1 of Exemplary Embodiments.

1. A method of reducing risk of future melanoma metastasis and/or progression in a subject with high-risk primary melanoma, including administering to the subject a composition including an inhibitor of HMGCR (a statin).

2. The method of embodiment 1, wherein primary melanoma is determined to be high-risk based on one or more genetic features.

3. The method of embodiment 2, wherein the genetic features include a mutation in any of the genes listed in Table 5.

4. The method of embodiment 2, wherein the genetic features include methylation level of any of the genes listed in Table 5.

5. The method of embodiment 2, wherein the genetic features include expression level of any of the genes listed in Table 5.

6. The method of any one of embodiments 3-5, wherein the genetic features are evaluated by gene expression profiling (GEP).

7. The method of embodiment 6, wherein the gene expression profile includes at least one of the genes listed in Table 5.

8. The method of embodiment 6, wherein the gene expression profile includes at least five of the genes listed in Table 5.

9. The method of embodiment 6, wherein the gene expression profile includes at least ten of the genes listed in Table 5.

10. The method of embodiment 6, wherein the gene expression profile includes at least twenty six (26) of the genes listed in Table 5.

11. The method of embodiment 6, wherein the gene expression profile includes at least 30, at least 40, at least 50, or at least 60, at least 70, at least 80, at least 90, at least 100, at least 125, or at least 150 of the genes listed in Table 5.

12. The method of embodiment 6, wherein the gene expression profile includes all of the genes listed in Table 5.

13. The method of embodiment 1, wherein subject is selected for treatment based on at least one high risk clinicopathologic features selected from the group consisting of Breslow depth, histologic subtype, mitotic rate, ulceration, sentinel lymph node status, and results of imaging.

14. The method of embodiment 1, wherein subject is selected for statin treatment based on a combination of clinicopathologic features and genetic features.

15. The method of embodiment 1, wherein the statin includes one or more of: fluvastatin, pitavastatin, atorvastatin, simvastatin, lovastatin, rosuvastatin, or pravastatin.

16. The method of embodiment 1, further including treating the subject with immunotherapy or chemotherapy or both.

17. The method according to embodiment 1, further including treating the subject with nicotinamide or niacin or both.

18. A method of selecting a subject afflicted with primary melanoma for treatment with a statin, the method including measurement of a gene expression profile in the primary melanoma.

19. The method of embodiment 18, wherein the gene expression profile is composed of measurement of levels of expression of one or more genes in Table 5.

20. The method of embodiment 18, wherein the gene expression profile includes at least one of the genes listed in Table 5.

21. The method of embodiment 18, wherein the gene expression profile includes at least five of the genes listed in Table 5.

22. The method of embodiment 18, wherein the gene expression profile includes at least ten of the genes listed in Table 5.

23. The method of embodiment 18, wherein the gene expression profile includes at least twenty six (26) of the genes listed in Table 5.

24. The method of embodiment 18, wherein the gene expression profile includes at least 30, at least 40, at least 50, or at least 60, at least 70, at least 80, at least 90, at least 100, at least 125, or at least 150 of the genes listed in Table 5.

25. The method of embodiment 18, wherein the gene expression profile includes all of the genes listed in Table 5.

26. The method of embodiment 18, wherein the gene expression signature includes any subset of genes in Table 5.

27. The method of embodiment 18, wherein the gene expression signature: includes the genes in Table 1; consists essentially of the genes in table 1; consists of the genes in Table 1; includes the genes in Table 2; consists essentially of the genes in table 2; consists of the genes in Table 2; includes the genes in Table 3; consists essentially of the genes in table 3; or consists of the genes in Table 3.

28. The method of any one of embodiments 18-27, further including treating the subject with a statin.

29. The method of embodiment 28, wherein the statin includes one or more of fluvastatin, pitavastatin, atorvastatin, simvastatin, lovastatin, rosuvastatin, or pravastatin.

30. The method of embodiment 28, further including treating the subject with immunotherapy or chemotherapy.

31. The method according to embodiment 28, further including treating the subject with nicotinamide or niacin.

32. A method of selecting a subject afflicted with melanoma for treatment with a molecule inhibiting activity of HMGCR enzyme, the method including measurement of a gene expression signature in the primary melanoma.

33. The method of any one of embodiments 18-32, wherein the subject afflicted with primary melanoma is selected for treatment with a statin in order to reduce likelihood of a future metastasis of the melanoma.

34. A method of selecting a melanoma patient for treatment with a statin (HMGCR inhibitor), including: obtaining a sample from a primary cutaneous melanoma tumor in the subject; measuring gene expression levels of at least two genes listed in Table 5 in the sample, wherein measuring gene expression levels includes measurement of a level of fluorescence by a sequence detection system following RT-PCR; producing a gene expression profile including the gene expression levels of the at least two genes; comparing the gene expression profile to a gene expression profile of a reference training set, which training set gene expression profile includes the same at least two genes; and providing an indication that the primary cutaneous melanoma tumor is low-risk or high-risk of metastasis when the gene expression profile indicates that expression levels of at least one gene is altered in a predictive manner as compared to the gene expression profile of the reference training set.

35. The method of embodiment 34, further including treating the patient with a statin or other HMGCR inhibitor when the primary cutaneous melanoma is determined to be at high-risk for metastasis.

### Set 2 of Exemplary Embodiments

1. A method of reducing risk of future melanoma metastasis and/or progression in a subject with high-risk primary melanoma, including: selecting a subject with high-risk primary melanoma, and administering to the selected subject a composition including an inhibitor of 3-Hydroxy-3-Methylglutaryl-CoA Reductase (HMGCR) (a statin).

2. The method of embodiment 1, wherein primary melanoma is determined to be high-risk based on one or more genetic features.

3. The method of embodiment 2 (or any of the above embodiments), wherein the genetic features include: a mutation in any of the genes listed in Table 5; or a change in expression of any of the genes listed in Table 5, compared to expression of the gene in a control or reference sample.

4. The method of embodiment 2 (or any of the above embodiments), wherein the genetic features include methylation level of any of the genes listed in Table 5.

5. The method of embodiment 2 (or any of the above embodiments), wherein the genetic features include expression level of any of the genes listed in Table 5.

6. The method of any one of embodiments 3-5, wherein the genetic features are evaluated by gene expression profiling (GEP).

7. The method of embodiment 6, wherein the gene expression profile includes at least one of the genes listed in Table 5.

8. The method of embodiment 6, wherein the gene expression profile includes at least five of the genes listed in Table 5.

9. The method of embodiment 6, wherein the gene expression profile includes at least ten of the genes listed in Table 5.

10. The method of embodiment 6, wherein the gene expression profile includes at least twenty six (26) of the genes listed in Table 5.

11. The method of embodiment 6, wherein the gene expression profile includes at least 30, at least 40, at least 50, or at least 60, at least 70, at least 80, at least 90, at least 100, at least 125, or more than 120 of the genes listed in Table 5.

12. The method of embodiment 6, wherein the gene expression profile includes all of the genes ANGPT2, ABCC12, ACOT1, ADAM12, AGBL4, AGXT, AIM2, ALK, ANGPT1, ANGPTL7, ANK1, AQP3, ARG1, ARRDC1, ART1, BAP1, BIN1, BMP2, BMX, BTG1, C8G, CACNG4, CAMK2B, CASQ1, CCR3, CCR5, CDC5L, CENPQ, CETP, CLCA2, CLIC5, COL24A1, CPN2, CRABP2, CST6, CTAGE1, CTCFL, CUL7, CXCL14, CXCL8/IL8, DIO3OS, DMAP1, DOCK3, DPEP3, DSC1, DYSF, EEF1A2, EHBP1L1, EIF1B, ERGIC2, F7, FASLG, FGF2, FLOT1, FLVCR2, FNBP1L, FREM2, GABBR2, GBP5, GCH1, GDF15, GFRA1, GJA1, GLDC, GPR39, GPR63, GRAMD1B, HEPACAM, HHATL, HMGCR, HNF4A, HTR3B, ID2, IFNG, ITGB3, JARID2, JPH3, KCNT1, KIF19, KLC4, KLHDC8A, KNDC1, KRT14, KRT6B, KRTAP19-6, KSR2, KYNU, LAG3, LAMB1, LGALSI, LGI4, LRRC31, LTA4H, LOXL4, MCF2, ME3, MFSD6L, MGP, MIR222, MLANA, MLF1, MTUS2, NFYA, NPPC, NTRK1, NXPH1, OCIAD2, OR52K1, OR5AK2, P2RY14, PARP11, PHEX, PIK3R6, PLA2G2D, PLAT, PLCZ1, PPL, PRF1, PRKCB, PROCA1, PTPRG, RAB15, RBBP4, RBM23, RGS7, RNF213, ROBO1, RP1, RPL7L1, S100A8, S100A9, S1PR1, SAP130, SERPINE2, SFMBT2, SIGLEC12, SLC17A3, SLC29A1, SLCO5A1, SLIT1, SOCS1, SOX4, SPAG6, SPP1, SPRR1B, STMN1, TACSTD2, TAS2R60, TDRD12, TFF2, TGFB1, TGFBRI, TLR4, TMCC3, TNFRSF10A, TP53, TRIM22, TRIM29, TRNAU1AP, TTN, TUBB, TYRP1, UTS2, VAMP5, VEGFA, VEGFC, VEGFD, WDR49, WIF1, ZIM2, ZNF560, and ZNF697.

13. The method of embodiment 6, wherein the gene expression profile: includes the genes in Table 1 (BAP1, MGP, SPP1, CXCL14, CLCA2, S100A8, BTG1, SAP130, ARG1, KRT6B, GJA1, ID2, EIF1B, S100A9, CRABP2, KRT14, ROBO1, RBM23, TACSTD2, DSC1, SPRR1B, TRIM29, AQP3, TYRP1, PPL, LTA4H, and CST6); consists essentially of the genes BAP1, MGP, SPP1, CXCL14, CLCA2, S100A8, BTG1, SAP130, ARG1, KRT6B, GJA1, ID2, EIF1B, S100A9, CRABP2, KRT14, ROBO1, RBM23, TACSTD2, DSC1, SPRR1B, TRIM29, AQP3, TYRP1, PPL, LTA4H, and CST6; consists of the genes in BAP1, MGP, SPP1, CXCL14, CLCA2, S100A8, BTG1, SAP130, ARG1, KRT6B, GJA1, ID2, EIF1B, S100A9, CRABP2, KRT14, ROBO1, RBM23, TACSTD2, DSC1, SPRR1B, TRIM29, AQP3, TYRP1, PPL, LTA4H, and CST6; includes the genes in Table 2 (CXCL8, ITGB3, LAMB1, PLAT, and TP53); consists essentially of the genes CXCL8, ITGB3, LAMB1, PLAT, and TP53; consists of the genes CXCL8, ITGB3, LAMB1, PLAT, and TP53; includes the genes in Table 3 (ANGPT1, ANGPT2, BMP2, FGF2, S1PR1, TGFB1, VEGFA, VEGFC, VEGFD, and IFNG); consists essentially of the genes ANGPT1, ANGPT2, BMP2, FGF2, S1PR1, TGFB1, VEGFA, VEGFC, VEGFD, and IFNG; consists of the genes ANGPT1, ANGPT2, BMP2, FGF2, S1 PR1, TGFB1, VEGFA, VEGFC, VEGFD, and IFNG; includes the genes in Table 4 (GDF15, MLANA, PLAT, CXCL8/IL8, ITGB3, LOXL4, PRKCB, SERPINE2, ADAM12, LGALSI, and TGFBRI); consists essentially of the genes GDF15, MLANA, PLAT, CXCL8/IL8, ITGB3, LOXL4, PRKCB, SERPINE2, ADAM12, LGALSI, and TGFBRI; or consists of the genes GDF15, MLANA, PLAT, CXCL8/IL8, ITGB3, LOXL4, PRKCB, SERPINE2, ADAM12, LGALSI, and TGFBR.

14. The method of embodiment 1 (or any of the above embodiments), wherein subject is selected for treatment based on at least one high risk clinicopathologic features selected from the group consisting of Breslow depth, histologic subtype, mitotic rate, ulceration, sentinel lymph node status, and results of imaging.

15. The method of embodiment 1 (or any of the above embodiments), wherein subject is selected for statin treatment based on a combination of clinicopathologic features and genetic features.

16. The method of embodiment 1 (or any of the above embodiments), wherein the statin includes one or more of: fluvastatin, pitavastatin, atorvastatin, simvastatin, lovastatin, rosuvastatin, or pravastatin.

17. The method of embodiment 1 (or any of the above embodiments), further including treating the subject with immunotherapy or chemotherapy or both.

18. The method according to embodiment 1 (or any of the above embodiments), further including treating the subject with nicotinamide or niacin or both.

19. A method of selecting a subject afflicted with primary melanoma for treatment with a statin, the method including measurement of a gene expression profile in the primary melanoma.

20. The method of embodiment 19, wherein the gene expression profile includes measurement of level(s) of expression of one or more genes in Table 5.

21. The method of embodiment 19, wherein the gene expression profile includes at least one of the genes listed in Table 5.

22. The method of embodiment 19, wherein the gene expression profile includes at least five of the genes listed in Table 5.

23. The method of embodiment 19, wherein the gene expression profile includes at least ten of the genes listed in Table 5.

24. The method of embodiment 19, wherein the gene expression profile includes at least twenty six (26) of the genes listed in Table 5.

25. The method of embodiment 19, wherein the gene expression profile includes at least 30, at least 40, at least 50, or at least 60, at least 70, at least 80, at least 90, at least 100, at least 125, or at least 150 of the genes listed in Table 5.

26. The method of embodiment 19, wherein the gene expression profile includes all of the genes listed in Table 5.

27. The method of embodiment 19, wherein the gene expression profile includes any subset of genes in Table 5.

28. The method of embodiment 19, wherein the gene expression profile: includes the genes in Table 1 (BAP1, MGP, SPP1, CXCL14, CLCA2, S100A8, BTG1, SAP130, ARG1, KRT6B, GJA1, ID2, EIF1B, S100A9, CRABP2, KRT14, ROBO1, RBM23, TACSTD2, DSC1, SPRR1B, TRIM29, AQP3, TYRP1, PPL, LTA4H, and CST6); consists essentially of the genes BAP1, MGP, SPP1, CXCL14, CLCA2, S100A8, BTG1, SAP130, ARG1, KRT6B, GJA1, ID2, EIF1B, S100A9, CRABP2, KRT14, ROBO1, RBM23, TACSTD2, DSC1, SPRR1 B, TRIM29, AQP3, TYRP1, PPL, LTA4H, and CST6; consists of the genes in BAP1, MGP, SPP1, CXCL14, CLCA2, S100A8, BTG1, SAP130, ARG1, KRT6B, GJA1, ID2, EIF1B, S100A9, CRABP2, KRT14, ROBO1, RBM23, TACSTD2, DSC1, SPRR1B, TRIM29, AQP3, TYRP1, PPL, LTA4H, and CST6; includes the genes in Table 2 (CXCL8, ITGB3, LAMB1, PLAT, and TP53); consists essentially of the genes CXCL8, ITGB3, LAMB1, PLAT, and TP53; consists of the genes CXCL8, ITGB3, LAMB1, PLAT, and TP53; includes the genes in Table 3 (ANGPT1, ANGPT2, BMP2, FGF2, S1PR1, TGFB1, VEGFA, VEGFC, VEGFD, and IFNG); consists essentially of the genes ANGPT1, ANGPT2, BMP2, FGF2, S1PR1, TGFB1, VEGFA, VEGFC, VEGFD, and IFNG; consists of the genes ANGPT1, ANGPT2, BMP2, FGF2, S1 PR1, TGFB1, VEGFA, VEGFC, VEGFD, and IFNG; includes the genes in Table 4 (GDF15, MLANA, PLAT, CXCL8/IL8, ITGB3, LOXL4, PRKCB, SERPINE2, ADAM12, LGALSI, and TGFBRI); consists essentially of the genes GDF15, MLANA, PLAT, CXCL8/IL8, ITGB3, LOXL4, PRKCB, SERPINE2, ADAM12, LGALSI, and TGFBRI; or consists of the genes GDF15, MLANA, PLAT, CXCL8/IL8, ITGB3, LOXL4, PRKCB, SERPINE2, ADAM12, LGALSI, and TGFBR.

29. The method of any one of embodiments 19-28, further including treating the subject with a statin.

30. The method of embodiment 29 (or any of embodiments 19-29), wherein the statin includes one or more of fluvastatin, pitavastatin, atorvastatin, simvastatin, lovastatin, rosuvastatin, or pravastatin.

31. The method of embodiment 29 (or any of embodiments 19-30), further including treating the subject with immunotherapy or chemotherapy.

32. The method according to embodiment 29 (or any of embodiments 19-31), further including treating the subject with nicotinamide or niacin.

33. A method of selecting a subject afflicted with melanoma for treatment with a molecule inhibiting activity of HMGCR enzyme, the method including measurement of a gene expression signature in the primary melanoma.

34. The method of any one of the embodiments, wherein the subject afflicted with primary melanoma is selected for treatment with a statin in order to reduce likelihood of a future metastasis of the melanoma.

35. The method of embodiment 29 (or any of embodiments 19-29), wherein the subject afflicted with primary melanoma is selected for treatment with a statin in order to reduce likelihood of a future metastasis of the melanoma.

36. A method of selecting a melanoma patient for treatment with a statin (HMGCR inhibitor), the method including: obtaining a sample from a primary cutaneous melanoma tumor in the subject; measuring gene expression levels of at least two genes listed in Table 5 in the sample; producing a gene expression profile including the gene expression levels of the at least two genes; comparing the gene expression profile to a gene expression profile of a reference training set gene expression profile, which training set gene expression profile includes the same at least two genes; and generating an indication that the primary cutaneous melanoma tumor is low-risk or high-risk of metastasis when the gene expression profile indicates that expression levels of at least one gene is altered in a predictive manner as compared to the gene expression profile of the reference training set.

37. The method of embodiment 36, wherein measuring gene expression levels includes measurement of a level of fluorescence by a sequence detection system following RT-PCR.

38. The method of embodiment 36 or 37, further including treating the patient with a statin or other HMGCR inhibitor when the primary cutaneous melanoma is determined to be at high-risk for metastasis.

39. The method of any one of embodiments 36-38, wherein the gene expression profile: includes the genes in Table 1 (BAP1, MGP, SPP1, CXCL14, CLCA2, S100A8, BTG1, SAP130, ARG1, KRT6B, GJA1, ID2, EIF1B, S100A9, CRABP2, KRT14, ROBO1, RBM23, TACSTD2, DSC1, SPRR1B, TRIM29, AQP3, TYRP1, PPL, LTA4H, and CST6); consists essentially of the genes BAP1, MGP, SPP1, CXCL14, CLCA2, S100A8, BTG1, SAP130, ARG1, KRT6B, GJA1, ID2, EIF1B, S100A9, CRABP2, KRT14, ROBO1, RBM23, TACSTD2, DSC1, SPRR1B, TRIM29, AQP3, TYRP1, PPL, LTA4H, and CST6; consists of the genes in BAP1, MGP, SPP1, CXCL14, CLCA2, S100A8, BTG1, SAP130, ARG1, KRT6B, GJA1, ID2, EIF1B, S100A9, CRABP2, KRT14, ROBO1, RBM23, TACSTD2, DSC1, SPRR1B, TRIM29, AQP3, TYRP1, PPL, LTA4H, and CST6; includes the genes in Table 2 (CXCL8, ITGB3, LAMB1, PLAT, and TP53); consists essentially of the genes CXCL8, ITGB3, LAMB1, PLAT, and TP53; consists of the genes CXCL8, ITGB3, LAMB1, PLAT, and TP53; includes the genes in Table 3 (ANGPT1, ANGPT2, BMP2, FGF2, S1PR1, TGFB1, VEGFA, VEGFC, VEGFD, and IFNG); consists essentially of the genes ANGPT1, ANGPT2, BMP2, FGF2, S1PR1, TGFB1, VEGFA, VEGFC, VEGFD, and IFNG; consists of the genes ANGPT1, ANGPT2, BMP2, FGF2, S1PR1, TGFB1, VEGFA, VEGFC, VEGFD, and IFNG; includes the genes in Table 4 (GDF15, MLANA, PLAT, CXCL8/IL8, ITGB3, LOXL4, PRKCB, SERPINE2, ADAM12, LGALSI, and TGFBRI); consists essentially of the genes GDF15, MLANA, PLAT, CXCL8/IL8, ITGB3, LOXL4, PRKCB, SERPINE2, ADAM12, LGALSI, and TGFBRI; or consists of the genes GDF15, MLANA, PLAT, CXCL8/IL8, ITGB3, LOXL4, PRKCB, SERPINE2, ADAM12, LGALSI, and TGFBR.

40. A method of treating melanoma in a patient having a primary melanoma tumor, the method including: administering to the patient a therapeutically effective dose of an inhibitor of 3-Hydroxy-3-Methylglutaryl-CoA Reductase (HMGCR) (a statin).

41. The method of embodiment 40, wherein the primary melanoma tumor is a Stage 3 (regional) or Stage 4 (metastatic) melanoma.

42. The method of embodiment 40 or 41, wherein the statin is fluvastatin, pitavastatin, atorvastatin, simvastatin, lovastatin, rosuvastatin, or pravastatin.

43. The method of any one of embodiments 40-42, further including: measuring gene expression levels of at least two genes selected from the genes listed in Table 5 in a sample of a primary cutaneous melanoma tumor in the patient; determining a patient gene-expression profile signature including the gene expression levels of the at least two genes; comparing the patient gene-expression profile signature to a gene-expression profile of a predictive training set; determining whether the patient gene-expression profile signature of the at least two genes is altered in a predictive manner compared to the predictive training set.

44. The method of any one of embodiments 40-43, wherein gene expression levels are measured using Polymerase Chain Reaction (PCR), Real-Time Polymerase Chain Reaction (RT-PCR), direct DNA expression in microarray, Sanger sequencing analysis, Northern blot, direct RNA expression detection serial analysis of gene expression, or next-generation RNA-sequencing.

45. The method of any one of embodiments 40-44, wherein the assay is RT-PCR.

46. The method of embodiment 40, wherein the gene expression profile includes at least five of the genes listed in Table 5.

47. The method of embodiment 40, wherein the gene expression profile includes at least ten of the genes listed in Table 5.

48. The method of embodiment 40, wherein the gene expression profile includes at least twenty six (26) of the genes listed in Table 5.

49. The method of embodiment 40, wherein the gene expression profile includes at least 30, at least 40, at least 50, or at least 60, at least 70, at least 80, at least 90, at least 100, at least 125, or at least 150 of the genes listed in Table 5.

50. The method of embodiment 40, wherein the gene expression profile includes all of the genes listed in Table 5.

51. The method of embodiment 40, wherein the gene expression profile: includes the genes in Table 1 (BAP1, MGP, SPP1, CXCL14, CLCA2, S100A8, BTG1, SAP130, ARG1, KRT6B, GJA1, ID2, EIF1B, S100A9, CRABP2, KRT14, ROBO1, RBM23, TACSTD2, DSC1, SPRR1B, TRIM29, AQP3, TYRP1, PPL, LTA4H, and CST6); consists essentially of the genes BAP1, MGP, SPP1, CXCL14, CLCA2, S100A8, BTG1, SAP130, ARG1, KRT6B, GJA1, ID2, EIF1B, S100A9, CRABP2, KRT14, ROBO1, RBM23, TACSTD2, DSC1, SPRR1 B, TRIM29, AQP3, TYRP1, PPL, LTA4H, and CST6; consists of the genes in BAP1, MGP, SPP1, CXCL14, CLCA2, S100A8, BTG1, SAP130, ARG1, KRT6B, GJA1, ID2, EIF1B, S100A9, CRABP2, KRT14, ROBO1, RBM23, TACSTD2, DSC1, SPRR1B, TRIM29, AQP3, TYRP1, PPL, LTA4H, and CST6; includes the genes in Table 2 (CXCL8, ITGB3, LAMB1, PLAT, and TP53); consists essentially of the genes CXCL8, ITGB3, LAMB1, PLAT, and TP53; consists of the genes CXCL8, ITGB3, LAMB1, PLAT, and TP53; includes the genes in Table 3 (ANGPT1, ANGPT2, BMP2, FGF2, S1PR1, TGFB1, VEGFA, VEGFC, VEGFD, and IFNG); consists essentially of the genes ANGPT1, ANGPT2, BMP2, FGF2, S1PR1, TGFB1, VEGFA, VEGFC, VEGFD, and IFNG; consists of the genes ANGPT1, ANGPT2, BMP2, FGF2, S1 PR1, TGFB1, VEGFA, VEGFC, VEGFD, and IFNG; includes the genes in Table 4 (GDF15, MLANA, PLAT, CXCL8/IL8, ITGB3, LOXL4, PRKCB, SERPINE2, ADAM12, LGALSI, and TGFBRI); consists essentially of the genes GDF15, MLANA, PLAT, CXCL8/IL8, ITGB3, LOXL4, PRKCB, SERPINE2, ADAM12, LGALSI, and TGFBRI; or consists of the genes GDF15, MLANA, PLAT, CXCL8/IL8, ITGB3, LOXL4, PRKCB, SERPINE2, ADAM12, LGALSI, and TGFBR.

52. A method of treating melanoma in a patient having a primary melanoma tumor, the method including administering to the patient: a therapeutically effective dose of an inhibitor of 3-Hydroxy-3-Methylglutaryl-CoA Reductase (HMGCR) (a statin); and a therapeutically effective dose of a melanoma treatment selected from a BRAF inhibitor and an immunotherapy for treatment of melanoma.

53. The method of embodiment 52, wherein the statin and the melanoma treatment are administered concurrently.

54. The method of embodiment 52 or 53, wherein the statin is fluvastatin, pitavastatin, atorvastatin, simvastatin, lovastatin, rosuvastatin, or pravastatin.

55. The method of any one of embodiments 52-54, wherein the BRAF inhibitor is Vemurafenib, dabrafenib, GDC-0879, PLX 4032, PLX-4720, PLX 4734, Sorafenib Tosylate, or a combination of trametinib and dabrafenib.

56. The method of any one of embodiments 1-55, wherein the subject is not treated with a RORgamma inhibitor.

### VIII. Experimental Examples

### Example 1: Statins as Modifiers of Prognostic Genetic Expression Signatures & Metastatic Behavior in Melanoma

This Example describes an in silico screen for drugs that reverse a “high-risk” gene expression profile associated with melanoma metastasis; Example 2 provides a more detailed discussion and analysis of the same research. At least some of the information described in this Example was published on or around Jan. 6, 2021, as Yu et al., J. Invest. Dermatol. 141:1802-1809, 2021.

By searching the Connectivity Map (cMap; Subamanian et al., Cell, 171(6)1437-1452, 2017; Lamb et al., Science 313(5795):1929-1935, 2006), a database of microarray gene expression measurements from over 27,000 pharmaceutical compounds, it was discovered that multiple members of the 3-hydroxy-3-methyl-glutaryl-CoA reductase (HMGCR) inhibitor drug class (statins) suppress gene expression profiles associated with metastasis.

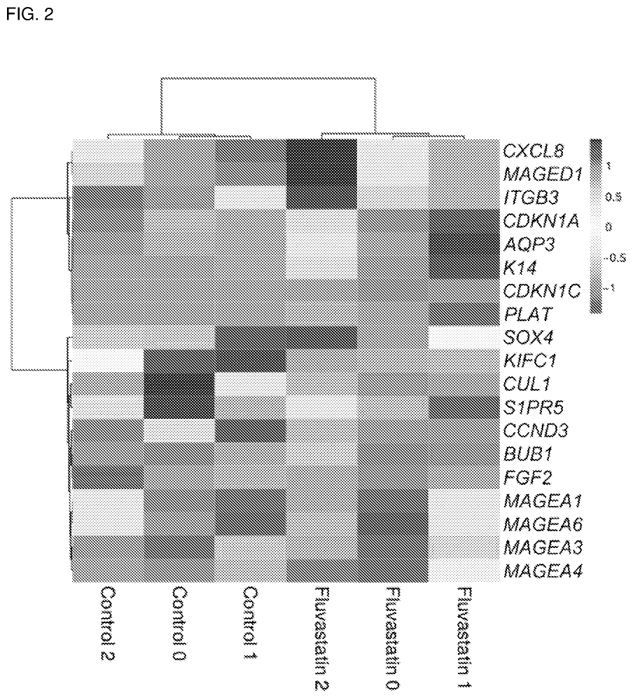

To validate the predicted effect of statins on melanoma gene expression, RNA-sequencing was used to measure changes in the transcriptome of A375 melanoma cells before and after treatment with fluvastatin. The data demonstrates that fluvastatin effectively reverses the metastatic gene expression profile of melanoma. Importantly, clinically relevant doses (3 μM concentration for 24 hours) were used, below the maximally tolerated dose of fluvastatin for these experiments. Fluvastatin significantly affected the expression of genes previously shown to be involved in metastasis (FIG. 2) (Li et al., Pigment Cell Melanoma Res. 2015. doi: 10.1111/pcmr.12374; Riker et al., BMC Med Genomics. 2008. doi: 10.1186/1755-8794-1-13; Brasseur et al., Int. J Cancer 63(3):375-380, 1995. doi: 10.1002/ijc.2910630313; Weon et al., Curr Opin Cell Biol. 2015. doi: 10.1016/j.ceb.2015.08.002; Jafarnejad et al., Oncogene. 2013. doi: 10.1038/onc.2012.239). Genes that drive lymphangiogenesis were also found be significantly decreased by fluvastatin treatment including FGF2, S1PR5, and TGFBRAP1 (Cao et al., Proc Natl Acad Sci USA. 2012. doi: 10.1073/pnas.1208324109; Huang et al. Biomolecules. 2013. doi: 10.3390/biom3030408; James et al. Dev. 2013. doi: 10.1242/dev.095026). Consistent with the hypothesized effect on melanoma metastasis, fluvastatin significantly altered the expression of genes included in melanoma prognostic tests (DecisionDx-Melanoma, Castle; Merlin Assay, SkylineDx), but did not alter expression of any genes used in a diagnostic test that distinguishes melanoma from nevi (myPath Melanoma, Myriad Genetics) (Clarke et al., J Cutan Pathol. 2015; 42(4):244-252. doi: 10.1111/cup.12475). This supports the hypothesis that the effect of statins is specific to progression and metastasis rather than tumor initiation.

Observations from a retrospective cohort of 475 patients with melanoma from our institution further indicate that statins may prevent metastasis. Patients taking statins had a 34% relative risk reduction (12.9% absolute risk reduction) for regional or distant metastasis at the time of melanoma diagnosis (24.7% taking statins vs 37.6% not taking statins, p=0.038). This result remained significant in multivariate analysis, after controlling for age, Breslow depth, ulceration, and mitotic rate (p=0.016, Table 6). Interestingly, in the cohort patients taking statins had thicker primary melanomas with higher mitotic count. The fact that these patients still had fewer metastases despite significantly worse primary tumors is remarkable.

TABLE 6
Multivariate analysis for factors that predispose
to metastasis at initial workup.

| Factors | Estimate | Standard error | p-value |
|---|---|---|---|
| Age (days) | −7.631 × 10⁻⁶ | 1.163 × 10⁻⁵ | 0.5118 |
| Depth of melanoma (mm) | 0.1054 | 0.05044 | 0.037 |
| Ulceration present | 0.2682 | 0.2742 | 0.328 |
| Dermal mitotic rate | 0.02826 | 0.03572 | 0.429 |
| Taking statin | −0.7580 | 0.3156 | 0.016 |

### Example 2: Computational Drug Repositioning Identifies Statins as Modifiers of Prognostic Genetic Expression Signatures and Metastatic Behavior in Melanoma

This example explores in greater detail the discovery that statin use causes differential expression in genes associated with melanoma metastasis, and that these changes in expression can be used clinically. At least some of the material described in this example was published in Yu et al. (J Invest. Dermatol. 141:1802-18-9, 2021; doi:10.1061/j.jid.2020.12.015; published online Jan. 6, 2021).

Despite advances in melanoma treatment, more than 70% of patients with distant metastasis die within 5 years. Proactive treatment of early melanoma to prevent metastasis could save lives and reduce overall healthcare costs. Currently, there are no treatments specifically designed to prevent early melanoma from progressing to metastasis. The Connectivity Map was used to conduct an in silico drug screen and identified 3-hydroxy-3-methyl-glutaryl-coenzyme A reductase inhibitors (statins) as a drug class that might prevent melanoma metastasis. To confirm the in vitro effect of statins, RNA sequencing was completed on A375 cells after treatment with fluvastatin to describe changes in the melanoma transcriptome. Statins induced differential expression in genes associated with metastasis and are used in commercially available prognostic tests for melanoma metastasis. Finally, a chart review of 475 patients with melanoma was completed. Patients taking statins were less likely to have metastasis at the time of melanoma diagnosis in both univariate and multivariate analyses (24.7% taking statins vs. 37.6% not taking statins, absolute risk reduction=12.9%, P=0.038). These findings suggest that statins might be useful as a treatment to prevent melanoma metastasis. Prospective trials are required to verify these findings and to determine the mechanism of metastasis prevention.

Metastasis is the primary driver of cancer mortality (Dillekas et al., Cancer Med 2019; 8:5574e69; Zbytek et al., Expert Rev Dermatol 2008; 3: 569e85). Despite recent advances in treatment, patients with metastatic melanoma survive on average <2 years after diagnosis (Kandel et al., Eur J Cancer 2018; 105:33e408; Larkin et al., N Eng/J Med 2019; 381:1535e; Robert et al., N Eng/J Med 2019; 381:626e36). In addition, the cost of treating metastatic disease has increased significantly (Kandel et al., Eur J Cancer 2018; 105:33). Preventing early cutaneous melanomas from progressing to metastasis may decrease healthcare costs and save lives.

Melanomas at high risk of metastasis can be identified by their gene expression signature (Gerami et al., J Am Acad Dermatol 2015a; 72:780e5.e3; Gerami et al., Clin Cancer Res 2015b; 21:175e83; Kashani-Sabet et al., Clin Cancer Res 2017; 23:6888e92; Kashani-Sabet et al., Clin Cancer Res 2009; 15:6987e92; Zager et al., BMC Cancer 2018; 18:130). Retrospective and prospective studies have identified a 28-gene expression signature as an independent predictor of metastasis (Gerami et al., J Am Acad Dermatol 2015a; 72:780e5.e3; Gerami et al., Clin Cancer Res 2015b; 21:175e; Greenhaw et al., J Am Acad Dermatol 2020; 83:745e53; Zager et al., BMC Cancer 2018; 18:130). The immediate clinical utility of these tests is controversial, and the functional role of these genes in metastasis remains elusive. However, the fact that these gene signatures identify melanomas with up to 22-fold higher odds of recurrence or metastasis means that they might yield insights into the metastatic process and could even lead to potential therapies (Chan & Tsao, JAMA Dermatol 2020; 156:949e51; Grossman et al., JAMA Dermatol 2020; 156:1004e11).

The Connectivity Map (cMap) is a publicly available database maintained by the Broad Institute that contains microarray gene expression measurements from over 27,000 pharmaceutical compounds (Lamb, Nat Rev Cancer 2007; 7:54e60; Lamb et al., Science 2006; 313:1929e35). This database can be queried to identify drugs that induce expression signatures either similar to or opposed to a specified profile. By screening the database for compounds that induce genetic expression patterns directly opposed to a disease signature, the database has been used successfully to identify drugs for computational drug repurposing (the process of discovering new indications for existing drugs) (Chen et al., Nat Commun 2017; 8:16022; Sirota et al., Sci Trans/Med 2011; 3:96ra77).

The cMap was used to screen for drugs that reverse a high-risk gene expression profile of melanoma that has been validated in clinical samples (Gerami et al., J Am Acad Dermatol 2015a; 72:780e5.e3; Greenhaw et al., Dermatol Surg 2018; 44:1494e500; Zager et al., BMC Cancer 2018; 18:130). 3-hydroxy-3-methylglutaryl-coenzyme A reductase inhibitors (statins) were identified as candidate agents to oppose the high-risk melanoma gene expression profile. Previous studies on statins in melanoma have focused on initiation or primary prevention and have had mixed results. Two large cardiovascular trials demonstrated a reduction in melanoma incidence with statin use, but this effect was not observed in the Women's Health Initiative or two Dutch epidemiologic studies (Jagtap et al., Cancer 2012; 118:5124e31; Rubins et al., N Eng/J Med 1999; 341:410e8; Splichal et al., Semin Thromb Hemost 2003; 29: 259e74). Two meta-analyses also demonstrated no reduction in melanoma incidence with statin use, and a randomized controlled trial of lovastatin for melanoma prevention did not identify any significant decreases in melanocytic atypia or other melanoma initiation markers (Bonovas et al., Eur J Epidemiol 2010; 25:29e35; Freeman et al., J Natl Cancer Inst 2006; 98:1538e46; Linden et al., Cancer Prev Res (Phila) 2014; 7:496e504). Recently, a Mendelian randomization analysis using the UK Biobank demonstrated that individuals with variants in the 3-hydroxy-3-methylglutaryl-coenzyme A reductase region, which represent proxies for statin use, had decreased overall cancer risk but did not reach statistical significance for any site-specific cancers (Carter et al., Elife 2020; 9: e57191).

Although results have been equivocal in melanoma initiation, there is more consistent evidence that statins may prevent melanoma progression and metastasis. Both in vitro and animal models have demonstrated potential mechanisms by which statins could prevent melanoma metastasis by decreasing tumor cell migration, decreasing cell adhesion, and increasing immunogenicity (Collisson et al., Mol Cancer Ther 2003; 2:941e8; Kidera et al., J Exp Clin Cancer Res 2010; 29:127; Pich et al., Front Immunol 2013; 4:62; Zanfardino et al., Int J Oncol 2013; 43:1763e70). One study observed decreased Breslow depth and metastasis rate with statin use, but the decrease in metastasis was not statistically significant as observed when multivariable analysis was conducted (Koomen et al., Eur J Cancer 2007; 43:2580e9). Another population-based study on all-cause mortality in patients with melanoma found a trend toward decreased hazard of death, particularly in men, but did not reach statistical significance (Livingstone et al., Cancer Med 2014; 3:1284e93).

In this study, an in silico drug screen is presented that suggests that statins might modify gene expression correlated with metastasis. Next-generation RNA sequencing (RNA-seq) was then conducted to characterize the direct effects of clinically relevant doses of fluvastatin on the melanoma transcriptome in vitro. Finally, the association of statin use with metastasis was explored in a retrospective cohort of cutaneous melanoma.

### Results

### Identification of Statins as a Potential Treatment for Metastasis Prevention

It was hypothesized that compounds that induced gene expression signatures opposite to that of metastatic melanoma would prevent metastasis. A search of the cMap database identified piroxicam, sotalol, acyclovir, zalcitabine, and simvastatin as potential therapeutic agents (Table 7). The ability of a drug to shift a gene expression signature is measured by its connectivity score (tau), which is a standardized measure ranging from −100 to 100. For each drug in the list of query results, the score corresponds to the fraction of reference gene sets that affect the 28 genes more strongly. The reference gene sets are generated from all reference signatures of drugs in the cMap database. A score of 90 indicates that only 10% of the reference gene sets showed stronger effects. In general, tau 2:90 is considered strong and should be considered a hypothesis for further study. Simvastatin had a score of 91. The score of all statins combined was also checked to ensure that these drugs as a class had a consistent effect. The statin class score was 84.9, which is strong for a class of drugs averaged together. Statins were selected for further study because of their proven long-term tolerability, benign side effect profile suitable for the intended clinical use as preventive drugs, and possible melanoma chemopreventive effects published in the literature.

TABLE 7
Candidate Drugs

| | Tau | | Class |
|---|---|---|---|
| Drug | Score | Drug Class | Score |
| Piroxicam | 99.93 | Cyclooxygenase inhibitor | −33.86 |
| Sotalol | 99.47 | β-Adrenergic receptor antagonist | −13.36 |
| Acyclovir | 99.44 | DNA polymerase inhibitor | 23.83 |
| Zalcitabine | 99.12 | Nucleoside reverse transcriptase | 97.48 |
| | | inhibitor | |
| Simvastatin | 91.05 | HMGCR inhibitors (statins) | 84.94 |

Abbreviation: HMGCR, 3-hydroxy-3-methyl glutaryl-coenzyme A reductase.

### Fluvastatin Alters the Gene Expression Profile of Melanoma

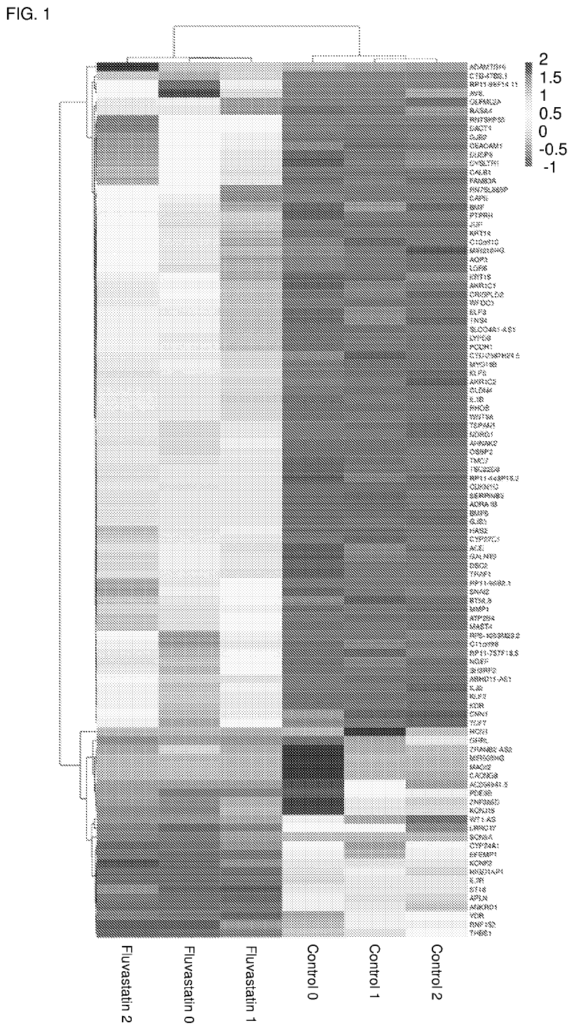

The cMap data are derived from the treatment of cell lines with statins at 10 mM concentration, which is above the maximal tolerated human dose (López-Aguilar et al., Arch Med Res 1999; 30:128e31; Tse et al., J Clin Pharmacol 1992; 32:630e8). Thus, the effect of statins on the melanoma transcriptome at clinically tolerable doses was characterized. RNA-seq was used to measure the gene expression of A375 melanoma cells before and after treatment with fluvastatin. The A375 cell line was specifically chosen because it has moderate metastatic potential and has been used in previous mechanistic studies of statins. Fluvastatin was chosen because of its lipophilicity (allowing extrahepatic distribution), benign side effect profile, and excellent bioavailability. 2,615 differentially expressed genes were identified (FIG. 1).

Fluvastatin significantly affected the expression of genes previously shown to be involved in metastasis, including MAGEA1, MAGEA3, MAGEA4, MAGEA6, MAGEDI, SOX4, BUB1, and KIFC1 (FIG. 2) (Brasseur et al., Int J Cancer 1995; 63:375e80; Jafarnejad et al., Oncogene 2013; 32:2131e9; Li et al., Pigment Cell Melanoma Res 2015; 28:453e63; Riker et al., BMC Med Genomics 2008; 1:13; Weon and Potts, Curr Opin Cell Biol 2015; 37:1e8). Genes that drive lymph angiogenesis were also found to be significantly decreased by fluvastatin treatment, including FGF2, S1 PR5, and TGFBRAP1 (Table 8) (Cao et al., Proc Natl Acad Sci USA 2012; 109:15894e9; Huang et al., Biomolecules 2013; 3:408e34; James et al., Development 2013; 140:3903e14).

TABLE 8
Genes of Interest Differentially Expressed
after Fluvastatin Treatment

| Gene Symbol | Log₂ Fold Change | q-Value |
|---|---|---|
| MAGEA1 | −0.4357 | 0.0018 |
| MAGEA3 | −0.4574 | 0.0018 |
| MAGEA4 | −0.2919 | 0.0407 |
| MAGEA6 | −0.4529 | 0.0018 |
| MAGED1 | 0.7222 | 0.0018 |
| SOX4 | −0.4139 | 0.0018 |
| BUB1 | −0.7319 | 0.0018 |
| KIFC1 | −0.4213 | 0.0355 |
| FGF2 | −0.5791 | 0.0018 |
| S1PR5 | −1.3212 | 0.0292 |
| TGFBRAP1 | −0.3389 | 0.0381 |
| CDKN1A | 1.3053 | 0.0018 |
| CDKN1C | 3.2643 | 0.0018 |
| CUL1 | −0.3847 | 0.0062 |
| CCND3 | −0.7194 | 0.0089 |
| ITGB3¹ | 0.5304 | 0.027 |
| AQP3² | 3.2162 | 0.0018 |
| K14² | 2.8530 | 0.0018 |
| PLAT¹ | 1.7389 | 0.0018 |
| CXCL8¹ | 0.8218 | 0.0018 |

Abbreviation: K, keratin.
¹SkylineDx profile.
²Castle Biosciences profile.

Consistent with the prediction that statins affect melanoma metastasis rather than initiation, fluvastatin significantly altered the expression of genes included in melanoma prognostic tests (DecisionDx-Melanoma, Castle Biosciences, Friendswood, TX; Merlin Assay, SkylineDx, Rotterdam, The Netherlands) but did not alter the expression of any genes used in a diagnostic test that distinguishes melanoma from nevi (myPath Melanoma, Myriad Genetics, Salt Lake City, UT) (Clarke et al., J Cutan Pathol 2015; 42:244e52). Genes included in these assays that were significantly shifted are presented in Table 8 (ITGB3; PLAT; CXCL; AQP3; and keratin 14 gene, K14). This suggests that the effect of statins is specific to progression and metastasis rather than to tumor initiation.

Gene ontology analysis of differentially expressed genes suggested significant enrichment of genes involved in the biological processes of cell proliferation, regulation of cell proliferation, tissue development, response to stimulus, and cell communication (all P<0.05). The molecular functions represented included signaling receptor activity, molecular transducer activity, receptor-ligand activity, receptor regulator activity, and transmembrane RSK binding (all P<0.05). Finally, the cellular components represented included plasma membrane, cell periphery, extracellular matrix, plasma membrane part, and extracellular region (all P<0.05).

Patients Taking Statins have a Significantly Lower Incidence of Metastasis at Diagnosis

To evaluate the clinical impact of statin use on melanoma metastasis, a retrospective cohort of 475 patients with melanoma were reviewed, of which 311 patients met the inclusion criteria. The mean age was 64.7 years. The mean Breslow depth in patients taking statins was 3.32 mm compared with 2.48 mm in those not taking statins (P=0.038) (Table 9). Regional or distant metastasis (defi as a positive completion of lymph node dissection or distant metastasis detected on imaging) was identified at diagnosis in 24.7% of patients taking statins and 37.6% of patients not taking statins (P=0.038). This result was significant in multivariate analysis after controlling for age, Breslow depth, ulceration, and mitotic rate (P=0.016) (Table 10).

TABLE 9
Demographics of Patients Taking and Not
Taking Statin at the Time of Biopsy

| | Taking | | Not Taking | | |
|---|---|---|---|---|---|
| | Statin | | Statin | | |
| Characteristics | (n = 77) | | (n = 234) | | P-Value |
| Mean age (y), n | 72.9 | | 61.5 | | <0.001 |
| Mean Breslow depth (mm), n | 3.32 | | 2.48 | | 0.038 |
| Ulceration, n (%) | | | | | |
| Yes | 26 | (33.8) | 79 | (33.8) | |
| No | 51 | (66.2) | 155 | (66.2) | 0.999 |
| Mitotic rate | | | | | |
| (mitoses/mm²), n (%) | | | | | |
| 0 | 9 | (11.7) | 28 | (11.9) | |
| 1-5 | 56 | (72.7) | 181 | (77.4) | |
| 6-10 | 5 | (6.5) | 21 | (9.0) | |
| >10 | 7 | (9.1) | 4 | (1.7) | 0.015 |
| Metastasis detected during | | | | | |
| initial workup, n (%) | | | | | |
| Yes | 19 | (24.7) | 88 | (37.6) | |
| No | 58 | (75.3) | 146 | (62.4) | 0.038 |

TABLE 10
Multivariate Analysis for Factors that Predispose
to Metastasis at Initial Workup

| Factors | OR (95% CI) | P-Value |
|---|---|---|
| Age (y) | 0.98 (0.97e1.00) | 0.01 |
| Depth (mm) | 1.12 (1.00e1.24) | 0.04 |
| Ulceration present | 1.44 (0.81e2.55) | 0.22 |
| Dermal mitoses (per mm²) | 1.02 (0.95e1.10) | 0.61 |
| Histologic type | | |
| Nodular | — | — |
| Superficial spreading | 0.93 (0.47e1.81) | 0.82 |
| Lentigo maligna | 0.27 (0.08e0.88) | 0.03 |
| Other | 0.81 (0.39e1.67) | 0.57 |
| Taking a statin | 0.48 (0.25e0.94) | 0.03 |

Abbreviation: CI, confidence interval.

### Discussion

Computational prediction has been successful in the past for identifying repurposing opportunities (Chen et al., Nat Commun 2017; 8:16022; Menden et al., Nat Commun 2019; 10:2674; Sirota et al., Sci Trans/Med 2011; 3:96ra77). In this study, an in silico screen was used to identify Food and Drug Administration-approved drugs that induce a genetic profile opposed to a validated gene expression profile that predicts melanoma metastasis. Statins were selected for further investigation on the basis of their long record of safety, their benign side effect profile consistent with their use as a preventive drug, and the literature suggestive of their potential activity in melanoma chemoprevention. Because the in silico screen uses data from experiments at doses above the maximal tolerated human serum concentration, the efficacy of statins in appropriately shifting the melanoma transcriptome at clinically achievable doses (3 mM) was verified.

It was found that fluvastatin caused significant changes in the melanoma transcriptome and affected the genes specific to melanoma metastasis at doses below the maximal tolerated dose (Lopez-Aguilar et al., Arch Med Res 1999; 30:128e31; Tse et al., J Clin Pharmacol 1992; 32:630e8). At these doses, fluvastatin did not affect the expression of genes that are used to differentiate nevi from melanoma, consistent with previous clinical trial results demonstrating no effect of statins on the progression of dysplastic nevi to melanoma (Linden et al., Cancer Prev Res (Phila) 2014; 7:496e504). However, fluvastatin influenced the expression of genes used to measure the risk of metastasis in commercially available tests, suggesting that the effect of statins is specific to melanoma progression and metastasis rather than to melanoma initiation. These data also imply that the history of statin use may be an important factor in interpreting the results of these prognostic tests. Because the drug concentrations used were lower than those in cMap (therapeutic rather than supratherapeutic) and because RNA-seq was used rather than microarray, there were fewer changes in the 28-gene expression profile than initially predicted by cMap. It was found that the RNA-seq validation experiments also revealed changes in genes outside of commercial tests that are known to influence metastatic potential and melanoma development.

These results suggest potential mechanisms for the effect of statins on melanoma metastasis. Regulation of the G1/S transition appeared to be affected by statin treatment. CDKN1A (p21) and CDKN1C (KIP2), which inhibit cell cycle progression in G1 and S phases, are typically suppressed in melanoma but had significantly increased expression after fluvastatin exposure (Jalili et al., J Natl Cancer Inst 2012; 104:1673e9; Yang et al., Cancer Cell Int 2020; 20: 32). The expression of CUL1 (Cullin 1), which promotes G1 to S phase transition and drives melanoma proliferation, was significantly decreased after fluvastatin treatment (Chen & Li, Int J Oncol 2010; 37: 1339e44). In addition, increased expression of CCND3 (Cyclin D3) has been shown to decrease survival and promote early relapse in melanoma (Florenes et al., Clin Cancer Res 2000; 6:3614e20). In this study, it was found that CCND3 expression was significantly decreased after fluvastatin treatment. KIFC1, a gene important in centrosome clustering, is overexpressed in primary and uveal melanoma cell lines as well as in breast and lung cancers (Pannu et al., Oncotarget 2015; 6: 6076e91). This study demonstrated that fluvastatin decreased the expression of KIFC1. Previous studies have demonstrated that metabolic differences in melanoma cells result in differences in metastatic potential (Tasdogan et al., Nature 2020; 577:115e20). SLC16A1 (MCT1), which has metabolic functions in lactate transport, has been shown to be an oncogene in malignant melanomas and neuroblastomas and to drive melanoma metastasis (Avitabile et al., Carcinogenesis 2020; 41:284e95). SLC16A1 downregulation was observed after fluvastatin exposure but did not achieve statistical significance after correction for multiple hypothesis testing (fold change=0.797, q=0.062). Lymphangiogenesis is thought to be involved in both metastasis and immune regulation of the tumor microenvironment (Lane et al., J Exp Med 2018; 215:3; Lund et al., Cell Rep 2012; 1:191e9; Lund et al., Cancer Discov 2016a; 6: 22e35; Lund et al., J Clin Invest 2016b; 126:3389e402). FGF2, S1PR5, and TGFBRAP1 are all involved in lymphangiogenesis and were downregulated by statin treatment. Finally, the MAGE gene family has been demonstrated to be expressed in a wide variety of malignancies, including melanoma, and is associated with increased invasion and metastasis (Barrow et al., Clin Cancer Res 2006; 12:764e71; Brasseur et al., Int J Cancer 1995; 63:375e80). Decreased expression of MAGEA1, MAGEA3, MAGEA4, and MAGEA6 was observed with fluvastatin treatment.

A previous study found that atorvastatin decreases isoprenylation of RhoC, thereby decreasing migration and invasion in a Matrigel transwell assay of A375 cells and metastasis in a mouse model (Collisson et al., Mol Cancer Ther 2003; 2:941 e8). The A375 cell line was chosen to build on this previous literature, and the data suggest that statins may also affect lymphangiogenesis, cell cycle regulation, and metabolism to reduce metastasis. The effect on cell cycle regulation identified in this study may be particularly relevant for the treatment of familial melanomas induced by CDKN2A mutations (Aspinwall et al., Cancer Epidemiol Biomarkers Prev 2008; 17: 1510e9; Goldstein et al., Cancer Res 2006; 66:9818e28; Goldstein et al., J Med Genet 2007; 44:99e106; Leachman et al., J Am Acad Dermatol 2009; 61:677.e1 e14).

It was considered that statins might induce gene expression changes that are correlated with metastasis but are not causative of metastasis. If this were true, statin use should not be correlated with the risk of metastasis. Thus, the association between statin use and metastasis was investigated in a retrospective cohort of patients with melanoma Patients taking statins at the time of biopsy were found to be significantly less likely to have metastasis at the time of melanoma diagnosis than those not taking statins, thereby suggesting that statins may be protective against melanoma metastasis. Statin use remained the strongest independent predictor of metastasis after correction for other prognostic factors, including depth, ulceration, mitoses, and age. The possibility was considered that statins may simply be a marker of better access to health care resulting in earlier melanoma diagnosis. However, the statin group in the described cohort actually had thicker primary melanomas with a higher mitotic count, indicating later diagnosis. The fact that these patients still had fewer metastases despite significantly worse primary tumors is remarkable.

The data in this stud are retrospective and correlative; despite controlling for all the prognostic factors available, it is possible that there are confounding variables beyond the researchers' knowledge, such as differences in patient behavior or access to health care. There is not enough follow-up data to determine for certain whether patients on statins have better future outcomes, although the reduction in metastases at diagnosis is promising. In addition, the study cohort is from a single tertiary referral center and thus may be biased toward a larger effect size than might be seen in a population-based study.

By leveraging a validated prognostic gene expression signature, statins were identified as a potential preventive therapy for melanoma metastasis, describe the effects of fluvastatin on the melanoma transcriptome, and demonstrate clinical activity in a retrospective cohort. Because the discovery of statins as potential prevention for metastasis was based on an existing commercial test, future clinical trials may be able to elegantly select the specific subset of patients who are most likely to benefit. Finally, as other genetic profiles are discovered, this tailored approach may identify additional drugs for the prevention or treatment of metastasis.

### Methods

### In Silico Selection of Candidates for Metastasis Prevention

The cMap Query Tool (https://clue.io/query) was used to conduct an in silico drug screen. The input query consisted of the 28 gene expression profiles from a commercially available prognostic test that predicts melanoma metastasis annotated by the desired change in expression (up or down) (Gerami et al., Clin Cancer Res 2015b; 21:175e83). Each compound and the corresponding drug class were scored for their ability to oppose the high-risk melanoma gene expression profile using the cMap connectivity score (tau), a standardized measure ranging from −100 to 100. The top 10% of drug candidates were then evaluated for Food and Drug Administration approval status and overall safety profile.

Characterization of the transcriptome in human melanoma cells exposed to fluvastatin The effect of statins on the melanoma transcriptome was characterized using a well-established melanoma cell line. Human melanoma cell line A375 (a generous gift of Dr John Letterio, Case Western Reserve University, Cleveland, OH) were maintained in standard growth media consisting of RPMI 1640 (Thermo Fisher Scientific, Waltham, MA) b 10% fetal bovine serum p 2 mM glutamine and grown in a 5% carbon dioxide-humidified atmosphere at 37° C. Cells were tested biannually and shown to be negative for mycoplasma contamination using the Mycoplasma Detection kit (MycoAlert, Lonza, Basel, Switzerland). For gene expression studies, A375 cells were seeded at 2.5E6 per 10 cm² dish and allowed to adhere overnight. Test samples (in triplicate) were treated with fluvastatin (purchased from Millipore Sigma, St. Louis, MO) at 3 mM concentration for 24 hours and then harvested for RNA extraction using the RNeasy Plus Mini Kit (catalog #74134; Qiagen, Germantown, MD) as per manufacturer's instructions. Untreated control cells grown side by side were used as the reference control for differential expression analysis. RNA was quantified using the Qubit Broad Range RNA kit (catalog #Q10210; Thermo Fisher Scientific) and diluted to 50 ng/ml for RNA-seq.

Sequencing reads generated from the Illumina HiSeq platform (Illumina, San Diego, CA) were assessed for quality and trimmed for adapter sequences using TrimGalore!, version 0.4.2 (Babraham Bioinformatics, Cambridge, United Kingdom), a wrapper script for FastQC and cutadapt. Reads that passed quality control were subsequently aligned to the human reference genome (GRCh38) using STAR aligner, version 2.5.1. Sequence alignment was guided using the GENCODE annotation for hg38. The aligned reads were analyzed for differential expression using Cufflinks, version 2.2.1, an RNA-seq analysis package that reports the fragments per kilobase of exon per million mapped for each gene. A differential analysis report was generated using the cuffdiff command performed in a pairwise manner for each group. Differential genes were identified using a significance cutoff of q<0.05. The differential expression profiles were then used as input in iPathwayGuide (Advaita Bioinformatics, Ann Arbor, MI) for pathway analysis.

### Multivariate Analysis of the Effect of Statin Use on Metastasis Incidence

To further understand how statins affect melanoma metastasis rates in the clinical setting, a retrospective chart review was performed of patients diagnosed with melanoma in the dermatopathology archive at the tertiary medical center from 1 Jan. 2007 through 31 Dec. 2017. This study was approved by Institutional Review Board. Patients with a histopathological diagnosis of melanoma with Breslow depth >0.8 mm or with ulceration were included. Patients with >3 primary melanomas were excluded to avoid confounding by patients with a germline predisposition to melanoma. Data collected included age at diagnosis of primary melanoma, sex, race, immunosuppression status (has a transplant, is HIV positive, has hematologic malignancy), statin use at the time of biopsy, histologic type, Breslow depth, ulceration, mitotic rate, tumor-infiltrating lymphocytes, regression, sentinel lymph node biopsy results, complete lymph node dissection results, and presence of metastasis at diagnosis. Univariate and multivariate analyses using logistic regression were performed to determine the relationship of statin use with the presence of metastasis at diagnosis, controlled for Breslow depth, ulceration status, and mitotic rate (glm function, R, version 4.0.2). A P-value of at least 0.05 was considered significant.

### Data Availability Statement

Datasets related to this article can be found online at ncbi.nlm. nih.gov/geo/, hosted at the National Center for Biotechnology Information Gene Expression Omnibus.

### Example 3. Retrospective Analysis of Statin Treatment of Melanomas

This example describes an analysis of the impact of statin treatment in patients with low-risk versus high-risk melanoma metastasis gene expression profiles (GEPs).

Data on melanomas treated at OHSU were retrospectively collected. Data included risk classification (high- or low-risk based on gene expression profile), statin use, and clinical outcomes (death, metastasis, recurrence). Progression free survival curves were plotted for patients with high-risk melanoma and low-risk melanoma stratified by statin use (survfit function in R version 3.5.3). Plots were visualized using ggplot2.

The resulting Kaplan-Meier curves shown in FIGS. 3A-3B clearly demonstrate that careful selection of patients for statin treatment is critical to their efficacy. FIG. 3A shows that statins are not effective at improving outcomes in GEP low-risk melanomas. This explains the failure of prior art to demonstrate any effect of statin treatment on melanoma since most melanomas are low-risk. The novel method described herein allows the selection of patients with a high risk GEP profile (FIG. 3B). In this group, statin therapy is highly effective and results in significantly longer progression and metastasis free survival.

### Example 4. Representative Clinical Uses

This Example provides a description of possible clinical uses that are enabled based on the teachings in this disclosure.

A patient with early melanoma (American Joint Committee on Cancer (AJCC) Stage 1-2) is diagnosed by skin biopsy. That melanoma specimen is tested by gene expression profiling (GEP) to determine whether it is a high-risk cancer (as described herein; specifically high risk for recurrence or metastasis).

Patients who have a “high risk” GEP are identified as appropriate for treatment with a statin regimen in order to reduce their risk of future melanoma metastasis and recurrence.

The clinical utility of this technology described herein is primarily in early stage melanoma, but it may also be extended to treatment of later stage melanoma by combining a statin with immunotherapy or other systemic treatments for later stage melanoma.

### Example 5. Statin Use is Associated with Improved Overall Survival in Patients with Melanoma

This Example describes analysis of a retrospective cohort, and demonstrates that statin use was strongly associated with increased overall survival, even after controlling for cancer stage, demographics, and comorbidities.

Melanoma continues to cause high mortality and healthcare cost (Kandel et al., EurJ Cancer. 2018. doi:10.1016/j.ejca.2018.09.026). This presents a clear need for additional cost-effective treatments for melanoma.

Current literature on statins in melanoma have focused on melanoma initiation rather than progression (Bonovas et al., Eur J Epidemiol. 2010. doi:10.1007/s10654-009-9396-x). In previously published work, it is demonstrated that 3-Hydroxy-3-Methylglutaryl-CoA Reductase (HMGCR) inhibitors (statins) influence tumor gene expression patterns and reduce metastasis in a retrospective patient cohort (Yu et al., J Invest Dermatol. 2021. doi:10.1016/j.jid.2020.12.015). The effect of statins on survival in melanoma patients is currently unknown. Statins are very low risk medications that might be ideal for preventing progression of high-risk resected melanoma or treatment of metastatic melanoma in addition to standard of care.

### Methods

Study Population: The Veterans Health Administration's (VA) Corporate Data Warehouse (CDW) contains individually identifiable clinical and demographic information from the 1990s through the present for over 19 million individual Veterans who received care provided or paid for by the VA. The CDW was searched for patients with a diagnosis of primary cutaneous malignant melanoma and who were receiving statin therapy at diagnosis. Patients were excluded if statin was discontinued before or initiated after diagnosis, or prescribed for less than one year in duration; statin non-users were never prescribed a statin. Patients with a diagnosis of other malignancies (except BCC or SCC) were excluded. Diagnoses were identified using International Classification of Diseases (ICD)-9 or ICD-10-Clinical Modification (CM) codes (Table 11), and therapeutic agents were identified by Current Procedural Terminology (CPT) codes and name using RxNorm. This was Institutional Review Board (IRB) approved.

TABLE 11
Diagnostic codes used for inclusion criteria

| System | Code | Description |
|---|---|---|
| ICD-9 | 172 | Malignant melanoma of skin of lip |
| ICD-9 | 172.1 | Malignant melanoma of skin of eyelid, including canthus |
| ICD-9 | 172.2 | Malignant melanoma of skin of ear and external auricular canal |
| ICD-9 | 172.3 | Malignant melanoma of other and unspecified part of face |
| ICD-9 | 172.4 | Malignant melanoma of scalp and neck |
| ICD-9 | 172.5 | Malignant melanoma of skin of trunk, except scrotum |
| ICD-9 | 172.6 | Malignant melanoma of skin of upper limb, including shoulder |
| ICD-9 | 172.7 | Malignant melanoma of skin of lower limb, including hip |
| ICD-9 | 172.8 | Malignant melanoma of other d sites of skin |
| ICD-9 | 172.9 | Malignant melanoma of skin, site unspecified |
| ICD-10 | C43.0 | Malignant melanoma of lip |
| ICD-10 | C43.10 | Malignant melanoma of unspecified eyelid, including canthus |
| ICD-10 | C43.11 | Malignant melanoma of right eyelid, including canthus |
| ICD-10 | C43.111 | Malignant melanoma of right upper eyelid, including canthus |
| ICD-10 | C43.112 | Malignant melanoma of right lower eyelid, including canthus |
| ICD-10 | C43.12 | Malignant melanoma of left eyelid, including canthus |
| ICD-10 | C43.121 | Malignant melanoma of left upper eyelid, including canthus |
| ICD-10 | C43.122 | Malignant melanoma of left lower eyelid, including canthus |
| ICD-10 | C43.20 | Malignant melanoma of unspecified ear and external auricular canal |
| ICD-10 | C43.21 | Malignant melanoma of right ear and external auricular canal |
| ICD-10 | C43.22 | Malignant melanoma of left ear and external auricular canal |
| ICD-10 | C43.30 | Malignant melanoma of unspecified part of face |
| ICD-10 | C43.31 | Malignant melanoma of nose |
| ICD-10 | C43.39 | Malignant melanoma of other parts of face |
| ICD-10 | C43.4 | Malignant melanoma of scalp and neck |
| ICD-10 | C43.51 | Malignant melanoma of anal skin |
| ICD-10 | C43.52 | Malignant melanoma of skin of breast |
| ICD-10 | C43.59 | Malignant melanoma of other part of trunk |
| ICD-10 | C43.60 | Malignant melanoma of unspecified upper limb, including shoulder |
| ICD-10 | C43.61 | Malignant melanoma of right upper limb, including shoulder |
| ICD-10 | C43.62 | Malignant melanoma of left upper limb, including shoulder |
| ICD-10 | C43.70 | Malignant melanoma of unspecified lower limb, including hip |
| ICD-10 | C43.71 | Malignant melanoma of right lower limb, including hip |
| ICD-10 | C43.72 | Malignant melanoma of left lower limb, including hip |
| ICD-10 | C43.8 | Malignant melanoma of overlapping sites of skin |
| ICD-10 | C43.9 | Malignant melanoma of skin, unspecified |

Covariates: Cancer stage was defined as local, regional, or distant, according to the most advanced stage found across four data sources: 1) diagnostic codes indicating nodal or metastatic disease; 2) Tumor (T), Node (N), and Metastasis (M) values extracted from relevant pathology reports; 3) natural language processing derived stage (Warner et al., J Oncol Pract. 2016; 12(2):157-158. doi:10.1200/JOP.2015.004622) from clinical notes from the first 90 days after diagnosis; and 4) raw VA cancer registry stage at diagnosis extracted from the CDW oncology domain.

Statistical Analyses: Kaplan Meier survival analyses were performed using the survminer package (version 0.4.8) for R (version 4.0.2). Risk was modeled using Cox proportional hazard regression including: melanoma stage, diagnosis year, Charlson score (Quan et al., Administrative Data. Med Care. 43(11):1130-1139, 2005; doi: 10.1097/01.mlr.0000182534.19832.83), patient's age at diagnosis, sex, body mass index (BMI), beta blocker use, and statin use. Data was censored at last recorded clinical follow-up.

TABLE 12
Cohort Characteristics

| | Statin n = 5,539 | | No Statin n = 12,442 | | p value |
|---|---|---|---|---|---|
| Local disease | 5,232 | (94.5%) | 11,344 | (91.2%) | p < 2.2e−16 |
| Regional disease | 25 | (0.5%) | 81 | (0.7%) | p = 0.084 |
| Distant disease | 282 | (5.1%) | 1,017 | (8.2%) | p = 1.1e−15 |
| Beta blocker use | 2,683 | (48.4%) | 737 | (5.9%) | p < 2.2e−16 |
| Male sex | 5,434 | (98.1%) | 12,020 | (96.6%) | p = 1.4e−09 |
| Age (years) | 75 ± 8.9 | (25-99) | 72 ± 13.3 | (18-106) | p < 2.2e−16 |
| median ± MAD (range) | | | | | |
| Year of diagnosis | 2008 ± 5.9 | (1998-2021) | 2010 ± 8.9 | (1990-2021) | p = 0.045 |
| median ± MAD (range) | | | | | |
| BMI | 29.1 ± 5.0 | (16-50) | 28.0 ± 4.9 | (15-50) | p < 2.2e−16 |
| mean ± SD (range) | | | | | |
| Charlson Score | 6.5 ± 2.2 | (1-20) | 5.4 ± 2.0 | (1-20) | p < 2.2e−16 |
| mean ± SD (range) | | | | | |

| | | | |
|---|---|---|---|
| Cholesterol mean ± SD | 164.5 ± 39.9 | 175.4 ± 38.3 | p < 2.2e−16 |
| Triglyceride mean ± SD | 145.9 ± 105.1 | 133.7 ± 92.3 | p = 6.8e−08 |
| HDL mean ± SD | 44.5 ± 12.8 | 47.1 ± 14.8 | p < 2.2e−16 |
| LDL mean ± SD | 92.5 ± 32.1 | 103.4 ± 32.8 | p < 2.2e−16 |

### Results

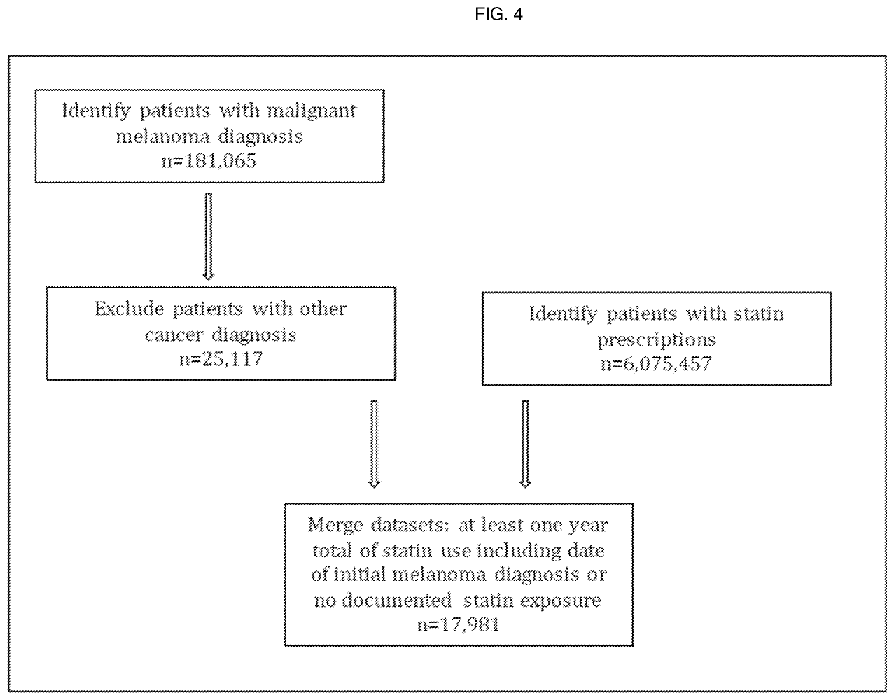

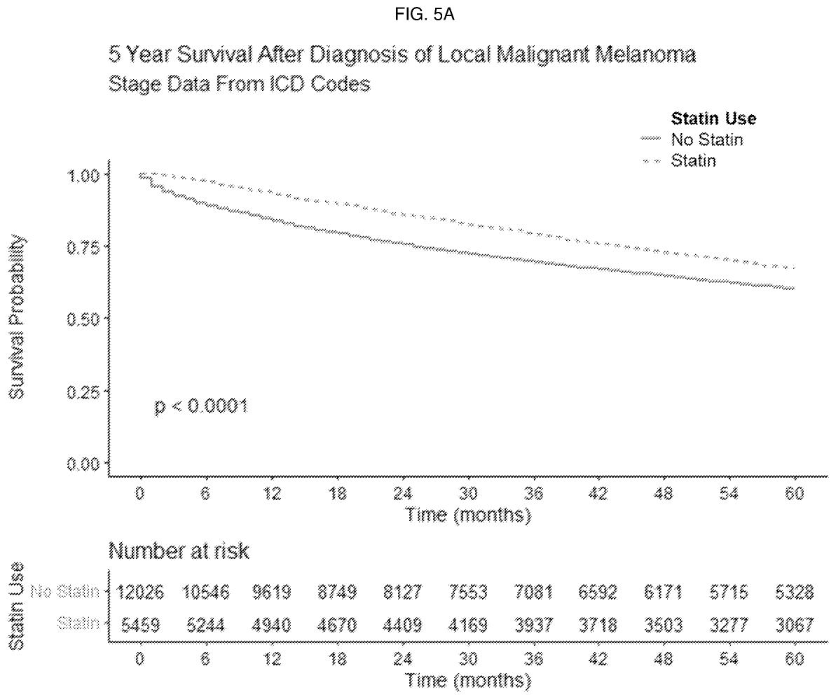

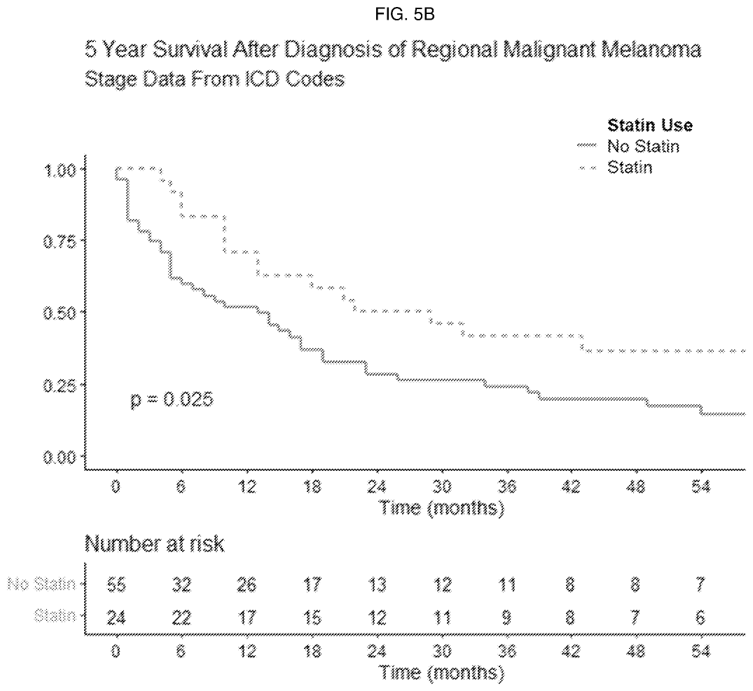

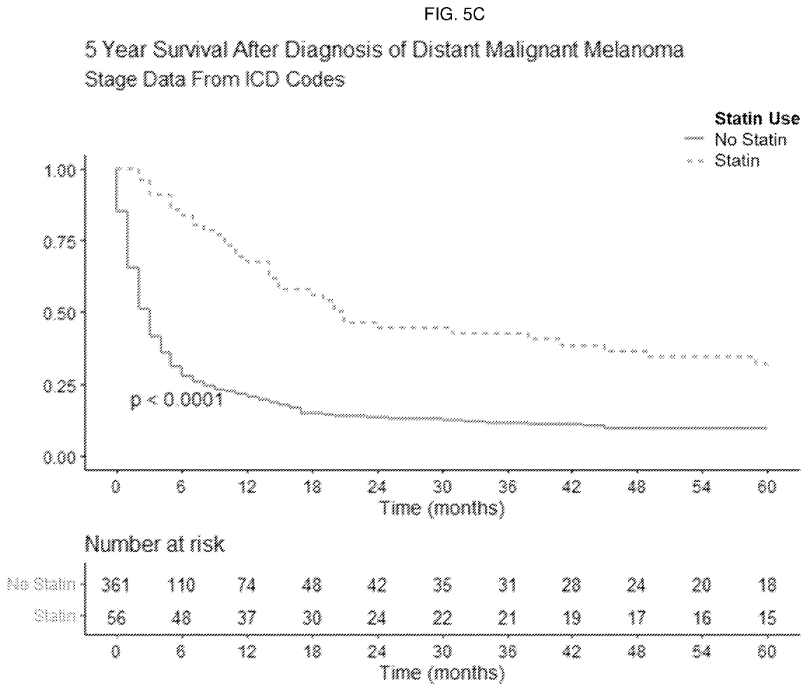

A total of 17,981 Veterans met inclusion criteria (FIG. 4). Average follow-up time was 5 years.

Statin users had better 5-year overall survival (OS) when compared to patients not taking statins when stratified by disease stage (local, regional, or distant disease). Comparisons of survival curves in each strata were significant at the 0.05 significance level (all p values<0.05). The effect of statins is most pronounced in more advanced stage disease, suggesting that clinical features such as Breslow depth, sentinel node status, mitoses, ulceration, and results of imaging may also be used to select patients for statin treatment.

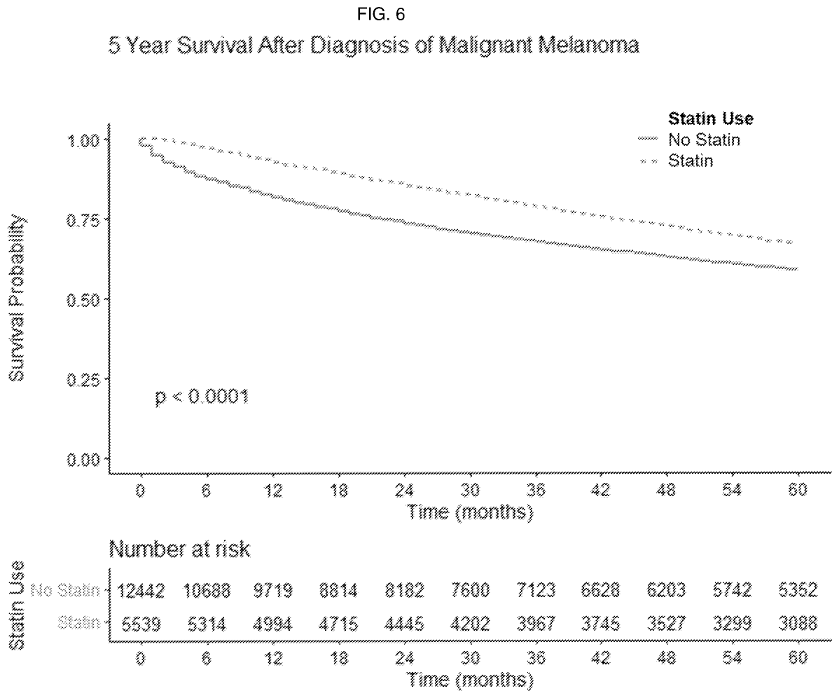

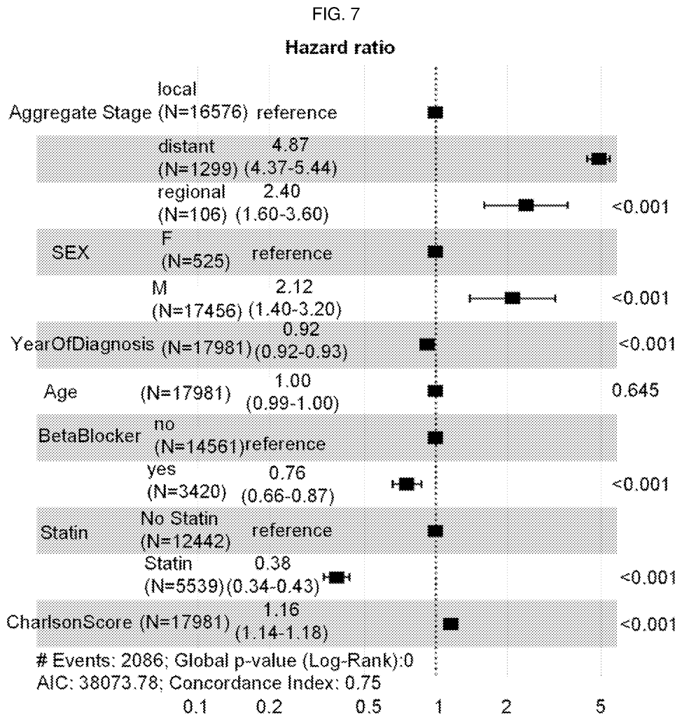

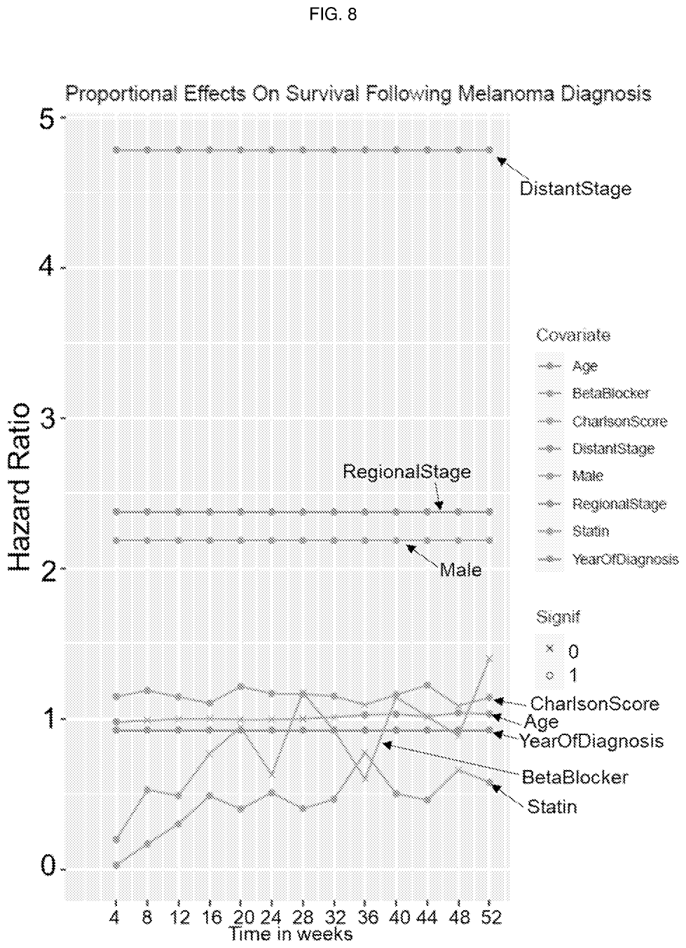

Statin users had better 5-year overall survival (OS) when compared to patients not taking statins (FIG. 6). This relationship remained significant in a multivariate model accounting for year of diagnosis, cancer stage, patient age, patient sex, BMI, use of beta blockers, and Charlson comorbidity score for the first year following diagnosis (FIG. 7; hazard ratio (HR) 0.38, 95% Cl 0.34-0.43 for statin user vs non-user).

### Discussion

In this retrospective cohort, statin use was strongly associated with increased OS, even after controlling for cancer stage, demographics, and comorbidities. Based on these results, statins are useful for treatment of Stage 3 (regional) and Stage 4 (metastatic) melanoma regardless of gene expression profile.

### Example 6. Three Year Survival after Initiation of Systemic Therapy for Patients with Malignant Melanoma

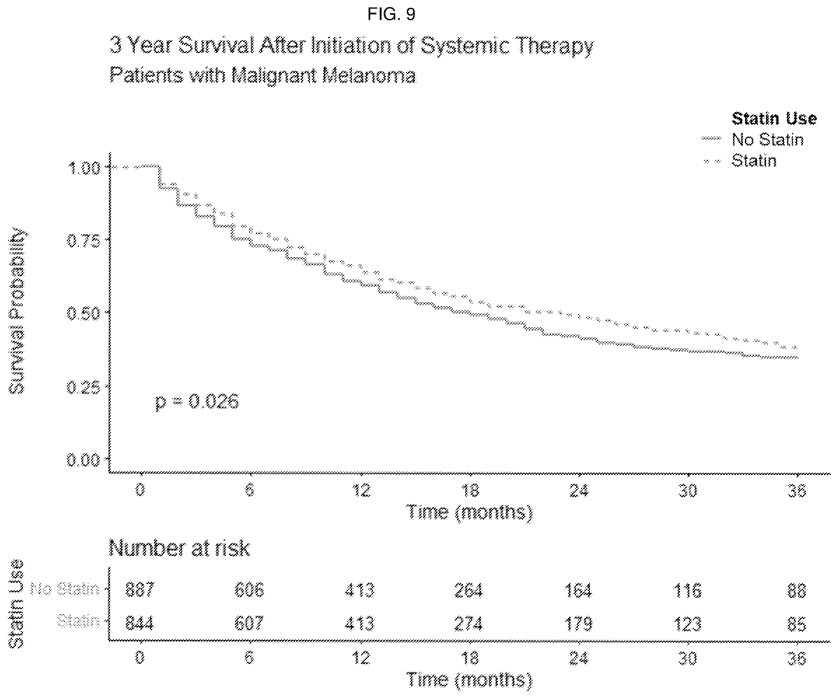

This example shows that combination treatment of statins with systemic therapy (either immunotherapy or BRAF inhibitors) leads to a clear increase in survival at 3 years (FIG. 9). The improvement of statin treated patients was significant at the 0.05 significance level.

The improvement is surprising since systemic therapy like BRAF inhibitors and immunotherapy are both directly targeting melanoma and are first line therapies, while statins have not previously been recommended by any clinical guidelines for treatment of melanoma. In addition, there are multiple ongoing clinical trials for metastatic melanoma attempting to improve responses to BRAF inhibitors and immunotherapy, none of which currently use statins. The findings reported here were statistically significant, and support use of statins in combination with either BRAF inhibitor therapy or immunotherapy for treatment of melanoma.

### IX. Closing Paragraphs

As will be understood by one of ordinary skill in the art, each embodiment disclosed herein can comprise, consist essentially of or consist of its particular stated element, step, ingredient or component. Thus, the terms “include” or “including” should be interpreted to recite: “comprise, consist of, or consist essentially of.” The transition term “comprise” or “comprises” means has, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the embodiment to the specified elements, steps, ingredients or components and to those that do not materially affect the embodiment. A material effect would cause a statistically significant change in measurement of a gene expression level (e.g., in a gene expression profile), and/or in identification of a subject as having a “high risk melanoma”, and/or in the success of treatment using a statin to reduce the risk of melanoma metastasis.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents, printed publications, journal articles, other written text, and web site content throughout this specification (referenced materials herein). Each of the referenced materials are individually incorporated herein by reference in their entirety for their referenced teaching(s), as of the filing date of the first application in the priority chain in which the specific reference was included. For instance, with regard to chemical compounds and nucleic acid or amino acids sequences referenced herein that are available in a public database, the information in the database entry is incorporated herein by reference as of the date that the database identifier was first included in the text of an application in the priority chain.

It is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

Definitions and explanations used in the present disclosure are meant and intended to be controlling in any future construction unless clearly and unambiguously modified in the example(s) or when application of the meaning renders any construction meaningless or essentially meaningless. In cases where the construction of the term would render it meaningless or essentially meaningless, the definition should be taken from Webster's Dictionary, 11th Edition or a dictionary known to those of ordinary skill in the art, such as the Oxford Dictionary of Biochemistry and Molecular Biology, 2^{nd} Edition (Ed. Anthony Smith, Oxford University Press, Oxford, 2006), and/or A Dictionary of Chemistry, 8^{th} Edition (Ed. J. Law & R. Rennie, Oxford University Press, 2020).

I claim:

1. A method of reducing risk of future melanoma metastasis and/or progression in a subject with high-risk primary melanoma, comprising:
selecting a subject with high-risk primary melanoma, and
administering to the selected subject a composition comprising an inhibitor of 3-Hydroxy-3-Methylglutaryl-CoA Reductase (HMGCR) (a statin).

2. The method of claim 1, wherein primary melanoma is determined to be high-risk based on one or more genetic features.

3. The method of claim 2, wherein the genetic features comprise:
a mutation in any of the genes listed in Table 5; or
a change in expression of any of the genes listed in Table 5, compared to expression of the gene in a control or reference sample.

4. The method of claim 2, wherein the genetic features comprise methylation level of any of the genes listed in Table 5 (ANGPT2, ABCC12, ACOT1, ADAM12, AGBL4, AGXT, AIM2, ALK, ANGPT1, ANGPTL7, ANK1, AQP3, ARG1, ARRDC1, ART1, BAP1, BIN1, BMP2, BMX, BTG1, C8G, CACNG4, CAMK2B, CASQ1, CCR3, CCR5, CDC5L, CENPQ, CETP, CLCA2, CLIC5, COL24A1, CPN2, CRABP2, CST6, CTAGE1, CTCFL, CUL7, CXCL14, CXCL8/IL8, DI030S, DMAP1, DOCK3, DPEP3, DSC1, DYSF, EEF1A2, EHBP1L1, EIF1B, ERGIC2, F7, FASLG, FGF2, FLOT1, FLVCR2, FNBP1L, FREM2, GABBR2, GBP5, GCH1, GDF15, GFRA1, GJA1, GLDC, GPR39, GPR63, GRAMD1B, HEPACAM, HHATL, HMGCR, HNF4A, HTR3B, ID2, IFNG, ITGB3, JARID2, JPH3, KCNT1, KIF19, KLC4, KLHDC8A, KNDC1, KRT14, KRT6B, KRTAP19-6, KSR2, KYNU, LAG3, LAMB1, LGALSI, LGI4, LRRC31, LTA4H, LOXL4, MCF2, ME3, MFSD6L, MGP, MIR222, MLANA, MLF1, MTUS2, NFYA, NPPC, NTRK1, NXPH1, OCIAD2, OR52K1, OR5AK2, P2RY14, PARP11, PHEX, PIK3R6, PLA2G2D, PLAT, PLCZ1, PPL, PRF1, PRKCB, PROCA1, PTPRG, RAB15, RBBP4, RBM23, RGS7, RNF213, ROBO1, RP1, RPL7L1, S100A8, S100A9, S1PR1, SAP130, SERPINE2, SFMBT2, SIGLEC12, SLC17A3, SLC29A1, SLCO5A1, SLIT1, SOCS1, SOX4, SPAG6, SPP1, SPRR1B, STMN1, TACSTD2, TAS2R60, TDRD12, TFF2, TGFB1, TGFBRI, TLR4, TMCC3, TNFRSF10A, TP53, TRIM22, TRIM29, TRNAU1AP, TTN, TUBB, TYRP1, UTS2, VAMP5, VEGFA, VEGFC, VEGFD, WDR49, WIF1, ZIM2, ZNF560, and ZNF697).

5. The method of claim 2, wherein the genetic features comprise expression level of any of the genes listed in Table 5 (ANGPT2, ABCC12, ACOT1, ADAM12, AGBL4, AGXT, AIM2, ALK, ANGPT1, ANGPTL7, ANK1, AQP3, ARG1, ARRDC1, ART1, BAP1, BIN1, BMP2, BMX, BTG1, C8G, CACNG4, CAMK2B, CASQ1, CCR3, CCR5, CDC5L, CENPQ, CETP, CLCA2, CLIC5, COL24A1, CPN2, CRABP2, CST6, CTAGE1, CTCFL, CUL7, CXCL14, CXCL8/I1L8, DI030S, DMAP1, DOCK3, DPEP3, DSC1, DYSF, EEF1A2, EHBP1L1, EIF1B, ERGIC2, F7, FASLG, FGF2, FLOT1, FLVCR2, FNBP1L, FREM2, GABBR2, GBP5, GCH1, GDF15, GFRA1, GJA1, GLDC, GPR39, GPR63, GRAMD1B, HEPACAM, HHATL, HMGCR, HNF4A, HTR3B, ID2, IFNG, ITGB3, JARID2, JPH3, KCNT1, KIF19, KLC4, KLHDC8A, KNDC1, KRT14, KRT6B, KRTAP19-6, KSR2, KYNU, LAG3, LAMB1, LGALSI, LGI4, LRRC31, LTA4H, LOXL4, MCF2, ME3, MFSD6L, MGP, MIR222, MLANA, MLF1, MTUS2, NFYA, NPPC, NTRK1, NXPH1, OCIAD2, OR52K1, OR5AK2, P2RY14, PARP11, PHEX, PIK3R6, PLA2G2D, PLAT, PLCZ1, PPL, PRF1, PRKCB, PROCA1, PTPRG, RAB15, RBBP4, RBM23, RGS7, RNF213, ROBO1, RP1, RPL7L1, S100A8, S100A9, S1PR1, SAP130, SERPINE2, SFMBT2, SIGLEC12, SLC17A3, SLC29A1, SLCO5A1, SLIT1, SOCS1, SOX4, SPAG6, SPP1, SPRR1B, STMN1, TACSTD2, TAS2R60, TDRD12, TFF2, TGFB1, TGFBRI, TLR4, TMCC3, TNFRSF10A, TP53, TRIM22, TRIM29, TRNAU1AP, TTN, TUBB, TYRP1, UTS2, VAMP5, VEGFA, VEGFC, VEGFD, WDR49, WIF1, ZIM2, ZNF560, and ZNF697).

6. The method of any one of claims 3-5, wherein the genetic features are evaluated by gene expression profiling (GEP).

7. The method of claim 6, wherein the gene expression profile comprises at least one of the genes listed in Table 5 (ANGPT2, ABCC12, ACOT1, ADAM12, AGBL4, AGXT, AIM2, ALK, ANGPT1, ANGPTL7, ANK1, AQP3, ARG1, ARRDC1, ART1, BAP1, BIN1, BMP2, BMX, BTG1, C8G, CACNG4, CAMK2B, CASQ1, CCR3, CCR5, CDC5L, CENPQ, CETP, CLCA2, CLIC5, COL24A1, CPN2, CRABP2, CST6, CTAGE1, CTCFL, CUL7, CXCL14, CXCL8/I1L8, DI030S, DMAP1, DOCK3, DPEP3, DSC1, DYSF, EEF1A2, EHBP1L1, EIF1B, ERGIC2, F7, FASLG, FGF2, FLOT1, FLVCR2, FNBP1L, FREM2, GABBR2, GBP5, GCH1, GDF15, GFRA1, GJA1, GLDC, GPR39, GPR63, GRAMD1B, HEPACAM, HHATL, HMGCR, HNF4A, HTR3B, ID2, IFNG, ITGB3, JARID2, JPH3, KCNT1, KIF19, KLC4, KLHDC8A, KNDC1, KRT14, KRT6B, KRTAP19-6, KSR2, KYNU, LAG3, LAMB1, LGALSI, LGI4, LRRC31, LTA4H, LOXL4, MCF2, ME3, MFSD6L, MGP, MIR222, MLANA, MLF1, MTUS2, NFYA, NPPC, NTRK1, NXPH1, OCIAD2, OR52K1, OR5AK2, P2RY14, PARP11, PHEX, PIK3R6, PLA2G2D, PLAT, PLCZ1, PPL, PRF1, PRKCB, PROCA1, PTPRG, RAB15, RBBP4, RBM23, RGS7, RNF213, ROBO1, RP1, RPL7L1, S100A8, S100A9, S1PR1, SAP130, SERPINE2, SFMBT2, SIGLEC12, SLC17A3, SLC29A1, SLCO5A1, SLIT1, SOCS1, SOX4, SPAG6, SPP1, SPRR1B, STMN1, TACSTD2, TAS2R60, TDRD12, TFF2, TGFB1, TGFBRI, TLR4, TMCC3, TNFRSF10A, TP53, TRIM22, TRIM29, TRNAU1AP, TTN, TUBB, TYRP1, UTS2, VAMP5, VEGFA, VEGFC, VEGFD, WDR49, WIF1, ZIM2, ZNF560, and ZNF697).

8. The method of claim 6, wherein the gene expression profile comprises at least five of the genes listed in Table 5 (ANGPT2, ABCC12, ACOT1, ADAM12, AGBL4, AGXT, AIM2, ALK, ANGPT1, ANGPTL7, ANK1, AQP3, ARG1, ARRDC1, ART1, BAP1, BIN1, BMP2, BMX, BTG1, C8G, CACNG4, CAMK2B, CASQ1, CCR3, CCR5, CDC5L, CENPQ, CETP, CLCA2, CLIC5, COL24A1, CPN2, CRABP2, CST6, CTAGE1, CTCFL, CUL7, CXCL14, CXCL8/I1L8, DI030S, DMAP1, DOCK3, DPEP3, DSC1, DYSF, EEF1A2, EHBP1L1, EIF1B, ERGIC2, F7, FASLG, FGF2, FLOT1, FLVCR2, FNBP1L, FREM2, GABBR2, GBP5, GCH1, GDF15, GFRA1, GJA1, GLDC, GPR39, GPR63, GRAMD1B, HEPACAM, HHATL, HMGCR, HNF4A, HTR3B, ID2, IFNG, ITGB3, JARID2, JPH3, KCNT1, KIF19, KLC4, KLHDC8A, KNDC1, KRT14, KRT6B, KRTAP19-6, KSR2, KYNU, LAG3, LAMB1, LGALSI, LGI4, LRRC31, LTA4H, LOXL4, MCF2, ME3, MFSD6L, MGP, MIR222, MLANA, MLF1, MTUS2, NFYA, NPPC, NTRK1, NXPH1, OCIAD2, OR52K1, OR5AK2, P2RY14, PARP11, PHEX, PIK3R6, PLA2G2D, PLAT, PLCZ1, PPL, PRF1, PRKCB, PROCA1, PTPRG, RAB15, RBBP4, RBM23, RGS7, RNF213, ROBO1, RP1, RPL7L1, S100A8, S100A9, S1PR1, SAP130, SERPINE2, SFMBT2, SIGLEC12, SLC17A3, SLC29A1, SLCO5A1, SLIT1, SOCS1, SOX4, SPAG6, SPP1, SPRR1B, STMN1, TACSTD2, TAS2R60, TDRD12, TFF2, TGFB1, TGFBRI, TLR4, TMCC3, TNFRSF10A, TP53, TRIM22, TRIM29, TRNAU1AP, TTN, TUBB, TYRP1, UTS2, VAMP5, VEGFA, VEGFC, VEGFD, WDR49, WIF1, ZIM2, ZNF560, and ZNF697).

9. The method of claim 6, wherein the gene expression profile comprises at least ten of the genes listed in Table 5 (ANGPT2, ABCC12, ACOT1, ADAM12, AGBL4, AGXT, AIM2, ALK, ANGPT1, ANGPTL7, ANK1, AQP3, ARG1, ARRDC1, ART1, BAP1, BIN1, BMP2, BMX, BTG1, C8G, CACNG4, CAMK2B, CASQ1, CCR3, CCR5, CDC5L, CENPQ, CETP, CLCA2, CLIC5, COL24A1, CPN2, CRABP2, CST6, CTAGE1, CTCFL, CUL7, CXCL14, CXCL8/I1L8, DI030S, DMAP1, DOCK3, DPEP3, DSC1, DYSF, EEF1A2, EHBP1L1, EIF1B, ERGIC2, F7, FASLG, FGF2, FLOT1, FLVCR2, FNBP1L, FREM2, GABBR2, GBP5, GCH1, GDF15, GFRA1, GJA1, GLDC, GPR39, GPR63, GRAMD1B, HEPACAM, HHATL, HMGCR, HNF4A, HTR3B, ID2, IFNG, ITGB3, JARID2, JPH3, KCNT1, KIF19, KLC4, KLHDC8A, KNDC1, KRT14, KRT6B, KRTAP19-6, KSR2, KYNU, LAG3, LAMB1, LGALSI, LGI4, LRRC31, LTA4H, LOXL4, MCF2, ME3, MFSD6L, MGP, MIR222, MLANA, MLF1, MTUS2, NFYA, NPPC, NTRK1, NXPH1, OCIAD2, OR52K1, OR5AK2, P2RY14, PARP11, PHEX, PIK3R6, PLA2G2D, PLAT, PLCZ1, PPL, PRF1, PRKCB, PROCA1, PTPRG, RAB15, RBBP4, RBM23, RGS7, RNF213, ROBO1, RP1, RPL7L1, S100A8, S100A9, S1PR1, SAP130, SERPINE2, SFMBT2, SIGLEC12, SLC17A3, SLC29A1, SLCO5A1, SLIT1, SOCS1, SOX4, SPAG6, SPP1, SPRR1B, STMN1, TACSTD2, TAS2R60, TDRD12, TFF2, TGFB1, TGFBRI, TLR4, TMCC3, TNFRSF10A, TP53, TRIM22, TRIM29, TRNAU1AP, TTN, TUBB, TYRP1, UTS2, VAMP5, VEGFA, VEGFC, VEGFD, WDR49, WIF1, ZIM2, ZNF560, and ZNF697).

10. The method of claim 6, wherein the gene expression profile comprises at least twenty six (26) of the genes listed in Table 5 (ANGPT2, ABCC12, ACOT1, ADAM12, AGBL4, AGXT, AIM2, ALK, ANGPT1, ANGPTL7, ANK1, AQP3, ARG1, ARRDC1, ART1, BAP1, BIN1, BMP2, BMX, BTG1, C8G, CACNG4, CAMK2B, CASQ1, CCR3, CCR5, CDC5L, CENPQ, CETP, CLCA2, CLIC5, COL24A1, CPN2, CRABP2, CST6, CTAGE1, CTCFL, CUL7, CXCL14, CXCL8/IL8, DI030S, DMAP1, DOCK3, DPEP3, DSC1, DYSF, EEF1A2, EHBP1L1, EIF1B, ERGIC2, F7, FASLG, FGF2, FLOT1, FLVCR2, FNBP1L, FREM2, GABBR2, GBP5, GCH1, GDF15, GFRA1, GJA1, GLDC, GPR39, GPR63, GRAMD1B, HEPACAM, HHATL, HMGCR, HNF4A, HTR3B, ID2, IFNG, ITGB3, JARID2, JPH3, KCNT1, KIF19, KLC4, KLHDC8A, KNDC1, KRT14, KRT6B, KRTAP19-6, KSR2, KYNU, LAG3, LAMB1, LGALSI, LGI4, LRRC31, LTA4H, LOXL4, MCF2, ME3, MFSD6L, MGP, MIR222, MLANA, MLF1, MTUS2, NFYA, NPPC, NTRK1, NXPH1, OCIAD2, OR52K1, OR5AK2, P2RY14, PARP11, PHEX, PIK3R6, PLA2G2D, PLAT, PLCZ1, PPL, PRF1, PRKCB, PROCA1, PTPRG, RAB15, RBBP4, RBM23, RGS7, RNF213, ROBO1, RP1, RPL7L1, S100A8, S100A9, S1PR1, SAP130, SERPINE2, SFMBT2, SIGLEC12, SLC17A3, SLC29A1, SLCO5A1, SLIT1, SOCS1, SOX4, SPAG6, SPP1, SPRR1B, STMN1, TACSTD2, TAS2R60, TDRD12, TFF2, TGFB1, TGFBRI, TLR4, TMCC3, TNFRSF10A, TP53, TRIM22, TRIM29, TRNAU1AP, TTN, TUBB, TYRP1, UTS2, VAMP5, VEGFA, VEGFC, VEGFD, WDR49, WIF1, ZIM2, ZNF560, and ZNF697).

11. The method of claim 6, wherein the gene expression profile comprises at least 30, at least 40, at least 50, or at least 60, at least 70, at least 80, at least 90, at least 100, at least 125, or more than 120 of the genes listed in Table 5.

12. The method of claim 6, wherein the gene expression profile comprises all of the genes ANGPT2, ABCC12, ACOT1, ADAM12, AGBL4, AGXT, AIM2, ALK, ANGPT1, ANGPTL7, ANK1, AQP3, ARG1, ARRDC1, ART1, BAP1, BIN1, BMP2, BMX, BTG1, C8G, CACNG4, CAMK2B, CASQ1, CCR3, CCR5, CDC5L, CENPQ, CETP, CLCA2, CLIC5, COL24A1, CPN2, CRABP2, CST6, CTAGE1, CTCFL, CUL7, CXCL14, CXCL8/I1L8, DIO3OS, DMAP1, DOCK3, DPEP3, DSC1, DYSF, EEF1A2, EHBP1L1, EIF1B, ERGIC2, F7, FASLG, FGF2, FLOT1, FLVCR2, FNBP1L, FREM2, GABBR2, GBP5, GCH1, GDF15, GFRA1, GJA1, GLDC, GPR39, GPR63, GRAMD1B, HEPACAM, HHATL, HMGCR, HNF4A, HTR3B, ID2, IFNG, ITGB3, JARID2, JPH3, KCNT1, KIF19, KLC4, KLHDC8A, KNDC1, KRT14, KRT6B, KRTAP19-6, KSR2, KYNU, LAG3, LAMB1, LGALSI, LGI4, LRRC31, LTA4H, LOXL4, MCF2, ME3, MFSD6L, MGP, MIR222, MLANA, MLF1, MTUS2, NFYA, NPPC, NTRK1, NXPH1, OCIAD2, OR52K1, OR5AK2, P2RY14, PARP11, PHEX, PIK3R6, PLA2G2D, PLAT, PLCZ1, PPL, PRF1, PRKCB, PROCA1, PTPRG, RAB15, RBBP4, RBM23, RGS7, RNF213, ROBO1, RP1, RPL7L1, S100A8, S100A9, S1PR1, SAP130, SERPINE2, SFMBT2, SIGLEC12, SLC17A3, SLC29A1, SLCO5A1, SLIT1, SOCS1, SOX4, SPAG6, SPP1, SPRR1B, STMN1, TACSTD2, TAS2R60, TDRD12, TFF2, TGFB1, TGFBRI, TLR4, TMCC3, TNFRSF10A, TP53, TRIM22, TRIM29, TRNAU1AP, TTN, TUBB, TYRP1, UTS2, VAMP5, VEGFA, VEGFC, VEGFD, WDR49, WIF1, ZIM2, ZNF560, and ZNF697.

13. The method of claim 6, wherein the gene expression profile:
comprises the genes in Table 1 (BAP1, MGP, SPP1, CXCL14, CLCA2, S100A8, BTG1, SAP130, ARG1, KRT6B, GJA1, ID2, EIF1B, S100A9, CRABP2, KRT14, ROBO1, RBM23, TACSTD2, DSC1, SPRR1B, TRIM29, AQP3, TYRP1, PPL, LTA4H, and CST6);
consists essentially of the genes BAP1, MGP, SPP1, CXCL14, CLCA2, S100A8, BTG1, SAP130, ARG1, KRT6B, GJA1, ID2, EIF1B, S100A9, CRABP2, KRT14, ROBO1, RBM23, TACSTD2, DSC1, SPRR1B, TRIM29, AQP3, TYRP1, PPL, LTA4H, and CST6;
consists of the genes in BAP1, MGP, SPP1, CXCL14, CLCA2, S100A8, BTG1, SAP130, ARG1, KRT6B, GJA1, ID2, EIF1B, S100A9, CRABP2, KRT14, ROBO1, RBM23, TACSTD2, DSC1, SPRR1B, TRIM29, AQP3, TYRP1, PPL, LTA4H, and CST6;
comprises the genes in Table 2 (CXCL8, ITGB3, LAMB1, PLAT, and TP53);
consists essentially of the genes CXCL8, ITGB3, LAMB1, PLAT, and TP53;
consists of the genes CXCL8, ITGB3, LAMB1, PLAT, and TP53;
comprises the genes in Table 3 (ANGPT1, ANGPT2, BMP2, FGF2, S1PR1, TGFB1, VEGFA, VEGFC, VEGFD, and IFNG);
consists essentially of the genes ANGPT1, ANGPT2, BMP2, FGF2, S1PR1, TGFB1, VEGFA, VEGFC, VEGFD, and IFNG;
consists of the genes ANGPT1, ANGPT2, BMP2, FGF2, S1PR1, TGFB1, VEGFA, VEGFC, VEGFD, and IFNG;
comprises the genes in Table 4 (GDF15, MLANA, PLAT, CXCL8/I1L8, ITGB3, LOXL4, PRKCB, SERPINE2, ADAM12, LGALSI, and TGFBRI);
consists essentially of the genes GDF15, MLANA, PLAT, CXCL8/I1L8, ITGB3, LOXL4, PRKCB, SERPINE2, ADAM12, LGALSI, and TGFBRI; or
consists of the genes GDF15, MLANA, PLAT, CXCL8/I1L8, ITGB3, LOXL4, PRKCB, SERPINE2, ADAM12, LGALSI, and TGFBR.

14. The method of claim 1, wherein subject is selected for treatment based on at least one high risk clinicopathologic features selected from the group consisting of Breslow depth, histologic subtype, mitotic rate, ulceration, sentinel lymph node status, and results of imaging.

15. The method of claim 1, wherein subject is selected for statin treatment based on a combination of clinicopathologic features and genetic features.

16. The method of claim 1, wherein the statin comprises one or more of: fluvastatin, pitavastatin, atorvastatin, simvastatin, lovastatin, rosuvastatin, or pravastatin.

17. The method of claim 1, further comprising treating the subject with immunotherapy or chemotherapy or both.

18. The method according to claim 1, further comprising treating the subject with nicotinamide or niacin or both.

19. A method of selecting a subject afflicted with primary melanoma for treatment with a statin, the method comprising measurement of a gene expression profile in the primary melanoma.

20. The method of claim 19, wherein the gene expression profile comprises measurement of level(s) of expression of one or more genes in Table 5 (ANGPT2, ABCC12, ACOT1, ADAM12, AGBL4, AGXT, AIM2, ALK, ANGPT1, ANGPTL7, ANK1, AQP3, ARG1, ARRDC1, ART1, BAP1, BIN1, BMP2, BMX, BTG1, C8G, CACNG4, CAMK2B, CASQ1, CCR3, CCR5, CDC5L, CENPQ, CETP, CLCA2, CLIC5, COL24A1, CPN2, CRABP2, CST6, CTAGE1, CTCFL, CUL7, CXCL14, CXCL8/I1L8, DI030S, DMAP1, DOCK3, DPEP3, DSC1, DYSF, EEF1A2, EHBP1L1, EIF1B, ERGIC2, F7, FASLG, FGF2, FLOT1, FLVCR2, FNBP1L, FREM2, GABBR2, GBP5, GCH1, GDF15, GFRA1, GJA1, GLDC, GPR39, GPR63, GRAMD1B, HEPACAM, HHATL, HMGCR, HNF4A, HTR3B, ID2, IFNG, ITGB3, JARID2, JPH3, KCNT1, KIF19, KLC4, KLHDC8A, KNDC1, KRT14, KRT6B, KRTAP19-6, KSR2, KYNU, LAG3, LAMB1, LGALSI, LGI4, LRRC31, LTA4H, LOXL4, MCF2, ME3, MFSD6L, MGP, MIR222, MLANA, MLF1, MTUS2, NFYA, NPPC, NTRK1, NXPH1, OCIAD2, OR52K1, OR5AK2, P2RY14, PARP11, PHEX, PIK3R6, PLA2G2D, PLAT, PLCZ1, PPL, PRF1, PRKCB, PROCA1, PTPRG, RAB15, RBBP4, RBM23, RGS7, RNF213, ROBO1, RP1, RPL7L1, S100A8, S100A9, S1PR1, SAP130, SERPINE2, SFMBT2, SIGLEC12, SLC17A3, SLC29A1, SLCO5A1, SLIT1, SOCS1, SOX4, SPAG6, SPP1, SPRR1B, STMN1, TACSTD2, TAS2R60, TDRD12, TFF2, TGFB1, TGFBRI, TLR4, TMCC3, TNFRSF10A, TP53, TRIM22, TRIM29, TRNAU1AP, TTN, TUBB, TYRP1, UTS2, VAMP5, VEGFA, VEGFC, VEGFD, WDR49, WIF1, ZIM2, ZNF560, and ZNF697).

21. The method of claim 19, wherein the gene expression profile comprises at least one of the genes listed in Table 5.

22. The method of claim 19, wherein the gene expression profile comprises at least five of the genes listed in Table 5.

23. The method of claim 19, wherein the gene expression profile comprises at least ten of the genes listed in Table 5.

24. The method of claim 19, wherein the gene expression profile comprises at least twenty six (26) of the genes listed in Table 5.

25. The method of claim 19, wherein the gene expression profile comprises at least 30, at least 40, at least 50, or at least 60, at least 70, at least 80, at least 90, at least 100, at least 125, or at least 150 of the genes listed in Table 5.

26. The method of claim 19, wherein the gene expression profile comprises all of the genes listed in Table 5.

27. The method of claim 19, wherein the gene expression profile comprises any subset of genes in Table 5.

28. The method of claim 19, wherein the gene expression profile:
comprises the genes in Table 1 (BAP1, MGP, SPP1, CXCL14, CLCA2, S100A8, BTG1, SAP130, ARG1, KRT6B, GJA1, ID2, EIF1B, S100A9, CRABP2, KRT14, ROBO1, RBM23, TACSTD2, DSC1, SPRR1B, TRIM29, AQP3, TYRP1, PPL, LTA4H, and CST6);
consists essentially of the genes BAP1, MGP, SPP1, CXCL14, CLCA2, S100A8, BTG1, SAP130, ARG1, KRT6B, GJA1, ID2, EIF1B, S100A9, CRABP2, KRT14, ROBO1, RBM23, TACSTD2, DSC1, SPRR1B, TRIM29, AQP3, TYRP1, PPL, LTA4H, and CST6;
consists of the genes in BAP1, MGP, SPP1, CXCL14, CLCA2, S100A8, BTG1, SAP130, ARG1, KRT6B, GJA1, ID2, EIF1B, S100A9, CRABP2, KRT14, ROBO1, RBM23, TACSTD2, DSC1, SPRR1B, TRIM29, AQP3, TYRP1, PPL, LTA4H, and CST6;
comprises the genes in Table 2 (CXCL8, ITGB3, LAMB1, PLAT, and TP53);
consists essentially of the genes CXCL8, ITGB3, LAMB1, PLAT, and TP53;
consists of the genes CXCL8, ITGB3, LAMB1, PLAT, and TP53;
comprises the genes in Table 3 (ANGPT1, ANGPT2, BMP2, FGF2, S1PR1, TGFB1, VEGFA, VEGFC, VEGFD, and IFNG);
consists essentially of the genes ANGPT1, ANGPT2, BMP2, FGF2, S1PR1, TGFB1, VEGFA, VEGFC, VEGFD, and IFNG;
consists of the genes ANGPT1, ANGPT2, BMP2, FGF2, S1PR1, TGFB1, VEGFA, VEGFC, VEGFD, and IFNG;
comprises the genes in Table 4 (GDF15, MLANA, PLAT, CXCL8/I1L8, ITGB3, LOXL4, PRKCB, SERPINE2, ADAM12, LGALSI, and TGFBRI);
consists essentially of the genes GDF15, MLANA, PLAT, CXCL8/I1L8, ITGB3, LOXL4, PRKCB, SERPINE2, ADAM12, LGALSI, and TGFBRI; or
consists of the genes GDF15, MLANA, PLAT, CXCL8/I1L8, ITGB3, LOXL4, PRKCB, SERPINE2, ADAM12, LGALSI, and TGFBR.

29. The method of any one of claims 19-28, further comprising treating the subject with a statin.

30. The method of claim 29, wherein the statin comprises one or more of fluvastatin, pitavastatin, atorvastatin, simvastatin, lovastatin, rosuvastatin, or pravastatin.

31. The method of claim 29, further comprising treating the subject with immunotherapy or chemotherapy.

32. The method according to claim 29, further comprising treating the subject with nicotinamide or niacin.

33. A method of selecting a subject afflicted with melanoma for treatment with a molecule inhibiting activity of HMGCR enzyme, the method comprising measurement of a gene expression signature in the primary melanoma.

34. The method of any one of claims 19-28 or 30-33, wherein the subject afflicted with primary melanoma is selected for treatment with a statin in order to reduce likelihood of a future metastasis of the melanoma.

35. The method of claim 29, wherein the subject afflicted with primary melanoma is selected for treatment with a statin in order to reduce likelihood of a future metastasis of the melanoma.

36. A method of selecting a melanoma patient for treatment with a statin (HMGCR inhibitor), the method comprising:
obtaining a sample from a primary cutaneous melanoma tumor in the subject;
measuring gene expression levels of at least two genes listed in Table 5 (ANGPT2, ABCC12, ACOT1, ADAM12, AGBL4, AGXT, AIM2, ALK, ANGPT1, ANGPTL7, ANK1, AQP3, ARG1, ARRDC1, ART1, BAP1, BIN1, BMP2, BMX, BTG1, C8G, CACNG4, CAMK2B, CASQ1, CCR3, CCR5, CDC5L, CENPQ, CETP, CLCA2, CLIC5, COL24A1, CPN2, CRABP2, CST6, CTAGE1, CTCFL, CUL7, CXCL14, CXCL8/IL8, DI030S, DMAP1, DOCK3, DPEP3, DSC1, DYSF, EEF1A2, EHBP1L1, EIF1B, ERGIC2, F7, FASLG, FGF2, FLOT1, FLVCR2, FNBP1L, FREM2, GABBR2, GBP5, GCH1, GDF15, GFRA1, GJA1, GLDC, GPR39, GPR63, GRAMD1B, HEPACAM, HHATL, HMGCR, HNF4A, HTR3B, ID2, IFNG, ITGB3, JARID2, JPH3, KCNT1, KIF19, KLC4, KLHDC8A, KNDC1, KRT14, KRT6B, KRTAP19-6, KSR2, KYNU, LAG3, LAMB1, LGALSI, LGI4, LRRC31, LTA4H, LOXL4, MCF2, ME3, MFSD6L, MGP, MIR222, MLANA, MLF1, MTUS2, NFYA, NPPC, NTRK1, NXPH1, OCIAD2, OR52K1, OR5AK2, P2RY14, PARP11, PHEX, PIK3R6, PLA2G2D, PLAT, PLCZ1, PPL, PRF1, PRKCB, PROCA1, PTPRG, RAB15, RBBP4, RBM23, RGS7, RNF213, ROBO1, RP1, RPL7L1, S100A8, S100A9, S1PR1, SAP130, SERPINE2, SFMBT2, SIGLEC12, SLC17A3, SLC29A1, SLCO5A1, SLIT1, SOCS1, SOX4, SPAG6, SPP1, SPRR1B, STMN1, TACSTD2, TAS2R60, TDRD12, TFF2, TGFB1, TGFBRI, TLR4, TMCC3, TNFRSF10A, TP53, TRIM22, TRIM29, TRNAU1AP, TTN, TUBB, TYRP1, UTS2, VAMP5, VEGFA, VEGFC, VEGFD, WDR49, WIF1, ZIM2, ZNF560, and ZNF697) in the sample;
producing a gene expression profile comprising the gene expression levels of the at least two genes;
comparing the gene expression profile to a gene expression profile of a reference training set gene expression profile, which training set gene expression profile comprises the same at least two genes; and
generating an indication that the primary cutaneous melanoma tumor is low-risk or high-risk of metastasis when the gene expression profile indicates that expression levels of at least one gene is altered in a predictive manner as compared to the gene expression profile of the reference training set.

37. The method of claim 36, wherein measuring gene expression levels comprises measurement of a level of fluorescence by a sequence detection system following RT-PCR.

38. The method of claim 36, further comprising treating the patient with a statin or other HMGCR inhibitor when the primary cutaneous melanoma is determined to be at high-risk for metastasis.

39. The method of claim 36, wherein the gene expression profile:
comprises the genes in Table 1 (BAP1, MGP, SPP1, CXCL14, CLCA2, S100A8, BTG1, SAP130, ARG1, KRT6B, GJA1, ID2, EIF1B, S100A9, CRABP2, KRT14, ROBO1, RBM23, TACSTD2, DSC1, SPRR1B, TRIM29, AQP3, TYRP1, PPL, LTA4H, and CST6);
consists essentially of the genes BAP1, MGP, SPP1, CXCL14, CLCA2, S100A8, BTG1, SAP130, ARG1, KRT6B, GJA1, ID2, EIF1B, S100A9, CRABP2, KRT14, ROBO1, RBM23, TACSTD2, DSC1, SPRR1B, TRIM29, AQP3, TYRP1, PPL, LTA4H, and CST6;
consists of the genes in BAP1, MGP, SPP1, CXCL14, CLCA2, S100A8, BTG1, SAP130, ARG1, KRT6B, GJA1, ID2, EIF1B, S100A9, CRABP2, KRT14, ROBO1, RBM23, TACSTD2, DSC1, SPRR1B, TRIM29, AQP3, TYRP1, PPL, LTA4H, and CST6;
comprises the genes in Table 2 (CXCL8, ITGB3, LAMB1, PLAT, and TP53);
consists essentially of the genes CXCL8, ITGB3, LAMB1, PLAT, and TP53;
consists of the genes CXCL8, ITGB3, LAMB1, PLAT, and TP53;
comprises the genes in Table 3 (ANGPT1, ANGPT2, BMP2, FGF2, S1PR1, TGFB1, VEGFA, VEGFC, VEGFD, and IFNG);
consists essentially of the genes ANGPT1, ANGPT2, BMP2, FGF2, S1PR1, TGFB1, VEGFA, VEGFC, VEGFD, and IFNG;
consists of the genes ANGPT1, ANGPT2, BMP2, FGF2, S1PR1, TGFB1, VEGFA, VEGFC, VEGFD, and IFNG;
comprises the genes in Table 4 (GDF15, MLANA, PLAT, CXCL8/IL8, ITGB3, LOXL4, PRKCB, SERPINE2, ADAM12, LGALSI, and TGFBRI);
consists essentially of the genes GDF15, MLANA, PLAT, CXCL8/IL8, ITGB3, LOXL4, PRKCB, SERPINE2, ADAM12, LGALSI, and TGFBRI; or
consists of the genes GDF15, MLANA, PLAT, CXCL8/IL8, ITGB3, LOXL4, PRKCB, SERPINE2, ADAM12, LGALSI, and TGFBR.

40. A method of treating melanoma in a patient having a primary melanoma tumor, the method comprising: administering to the patient a therapeutically effective dose of an inhibitor of 3-Hydroxy-3-Methylglutaryl-CoA Reductase (HMGCR) (a statin).

41. The method of claim 40, wherein the primary melanoma tumor is a Stage 3 (regional) or Stage 4 (metastatic) melanoma.

42. The method of claim 40, wherein the statin is fluvastatin, pitavastatin, atorvastatin, simvastatin, lovastatin, rosuvastatin, or pravastatin.

43. The method of claim 40, further comprising:
measuring gene expression levels of at least two genes selected from the genes listed in Table 5 in a sample of a primary cutaneous melanoma tumor in the patient;
determining a patient gene-expression profile signature comprising the gene expression levels of the at least two genes;
comparing the patient gene-expression profile signature to a gene-expression profile of a predictive training set;
determining whether the patient gene-expression profile signature of the at least two genes is altered in a predictive manner compared to the predictive training set.

44. The method of claim 43, wherein gene expression levels are measured using Polymerase Chain Reaction (PCR), Real-Time Polymerase Chain Reaction (RT-PCR), direct DNA expression in microarray, Sanger sequencing analysis, Northern blot, direct RNA expression detection serial analysis of gene expression, or next-generation RNA-sequencing.

45. The method of claim 44, wherein the assay is RT-PCR.

46. The method of claim 40, wherein the gene expression profile comprises at least five of the genes listed in Table 5 (ANGPT2, ABCC12, ACOT1, ADAM12, AGBL4, AGXT, AIM2, ALK, ANGPT1, ANGPTL7, ANK1, AQP3, ARG1, ARRDC1, ART1, BAP1, BIN1, BMP2, BMX, BTG1, C8G, CACNG4, CAMK2B, CASQ1, CCR3, CCR5, CDC5L, CENPQ, CETP, CLCA2, CLIC5, COL24A1, CPN2, CRABP2, CST6, CTAGE1, CTCFL, CUL7, CXCL14, CXCL8/IL8, DIO3OS, DMAP1, DOCK3, DPEP3, DSC1, DYSF, EEF1A2, EHBP1L1, EIF1B, ERGIC2, F7, FASLG, FGF2, FLOT1, FLVCR2, FNBP1L, FREM2, GABBR2, GBP5, GCH1, GDF15, GFRA1, GJA1, GLDC, GPR39, GPR63, GRAMD1B, HEPACAM, HHATL, HMGCR, HNF4A, HTR3B, ID2, IFNG, ITGB3, JARID2, JPH3, KCNT1, KIF19, KLC4, KLHDC8A, KNDC1, KRT14, KRT6B, KRTAP19-6, KSR2, KYNU, LAG3, LAMB1, LGALSI, LGI4, LRRC31, LTA4H, LOXL4, MCF2, ME3, MFSD6L, MGP, MIR222, MLANA, MLF1, MTUS2, NFYA, NPPC, NTRK1, NXPH1, OCIAD2, OR52K1, OR5AK2, P2RY14, PARP11, PHEX, PIK3R6, PLA2G2D, PLAT, PLCZ1, PPL, PRF1, PRKCB, PROCA1, PTPRG, RAB15, RBBP4, RBM23, RGS7, RNF213, ROBO1, RP1, RPL7L1, S100A8, S100A9, S1PR1, SAP130, SERPINE2, SFMBT2, SIGLEC12, SLC17A3, SLC29A1, SLCO5A1, SLIT1, SOCS1, SOX4, SPAG6, SPP1, SPRR1B, STMN1, TACSTD2, TAS2R60, TDRD12, TFF2, TGFB1, TGFBRI, TLR4, TMCC3, TNFRSF10A, TP53, TRIM22, TRIM29, TRNAU1AP, TTN, TUBB, TYRP1, UTS2, VAMP5, VEGFA, VEGFC, VEGFD, WDR49, WIF1, ZIM2, ZNF560, and ZNF697).

47. The method of claim 40, wherein the gene expression profile comprises at least ten of the genes listed in Table 5.

48. The method of claim 40, wherein the gene expression profile comprises at least twenty six (26) of the genes listed in Table 5.

49. The method of claim 40, wherein the gene expression profile comprises at least 30, at least 40, at least 50, or at least 60, at least 70, at least 80, at least 90, at least 100, at least 125, or at least 150 of the genes listed in Table 5.

50. The method of claim 40, wherein the gene expression profile comprises all of the genes listed in Table 5.

51. The method of claim 40, wherein the gene expression profile:
comprises the genes in Table 1 (BAP1, MGP, SPP1, CXCL14, CLCA2, S100A8, BTG1, SAP130, ARG1, KRT6B, GJA1, ID2, EIF1B, S100A9, CRABP2, KRT14, ROBO1, RBM23, TACSTD2, DSC1, SPRR1B, TRIM29, AQP3, TYRP1, PPL, LTA4H, and CST6);
consists essentially of the genes BAP1, MGP, SPP1, CXCL14, CLCA2, S100A8, BTG1, SAP130, ARG1, KRT6B, GJA1, ID2, EIF1B, S100A9, CRABP2, KRT14, ROBO1, RBM23, TACSTD2, DSC1, SPRR1B, TRIM29, AQP3, TYRP1, PPL, LTA4H, and CST6;
consists of the genes in BAP1, MGP, SPP1, CXCL14, CLCA2, S100A8, BTG1, SAP130, ARG1, KRT6B, GJA1, ID2, EIF1B, S100A9, CRABP2, KRT14, ROBO1, RBM23, TACSTD2, DSC1, SPRR1B, TRIM29, AQP3, TYRP1, PPL, LTA4H, and CST6;
comprises the genes in Table 2 (CXCL8, ITGB3, LAMB1, PLAT, and TP53);
consists essentially of the genes CXCL8, ITGB3, LAMB1, PLAT, and TP53;
consists of the genes CXCL8, ITGB3, LAMB1, PLAT, and TP53;
comprises the genes in Table 3 (ANGPT1, ANGPT2, BMP2, FGF2, S1PR1, TGFB1, VEGFA, VEGFC, VEGFD, and IFNG);
consists essentially of the genes ANGPT1, ANGPT2, BMP2, FGF2, S1PR1, TGFB1, VEGFA, VEGFC, VEGFD, and IFNG;
consists of the genes ANGPT1, ANGPT2, BMP2, FGF2, S1PR1, TGFB1, VEGFA, VEGFC, VEGFD, and IFNG;
comprises the genes in Table 4 (GDF15, MLANA, PLAT, CXCL8/I1L8, ITGB3, LOXL4, PRKCB, SERPINE2, ADAM12, LGALSI, and TGFBRI);
consists essentially of the genes GDF15, MLANA, PLAT, CXCL8/I1L8, ITGB3, LOXL4, PRKCB, SERPINE2, ADAM12, LGALSI, and TGFBRI; or
consists of the genes GDF15, MLANA, PLAT, CXCL8/I1L8, ITGB3, LOXL4, PRKCB, SERPINE2, ADAM12, LGALSI, and TGFBR.

52. A method of treating melanoma in a patient having a primary melanoma tumor, the method comprising administering to the patient:
a therapeutically effective dose of an inhibitor of 3-Hydroxy-3-Methylglutaryl-CoA Reductase (HMGCR) (a statin); and
a therapeutically effective dose of a melanoma treatment selected from a BRAF inhibitor and an immunotherapy for treatment of melanoma.

53. The method of claim 52, wherein the statin and the melanoma treatment are administered concurrently.

54. The method of claim 52, wherein the statin is fluvastatin, pitavastatin, atorvastatin, simvastatin, lovastatin, rosuvastatin, or pravastatin.

55. The method of claim 52, wherein the BRAF inhibitor is Vemurafenib, dabrafenib, GDC-0879, PLX 4032, PLX-4720, PLX 4734, Sorafenib Tosylate, or a combination of trametinib and dabrafenib.

56. The method of any one of claims 1-55, wherein the subject is not treated with a RORgamma inhibitor.
